SEQUENCE LISTING (83 sequences)

SEQ ID NO: 1 - DNA, 2685 bp, synthetic construct
  misc_feature 1..2685: note = Synthetic sequence, modified Cas3 with NLS
  source 1..2685: mol_type = other DNA
atgcccaagaagaagcggaaggtggaaccttttaaatatatatgccattactggggaaaatcctcaaaaagcttgacgaaaggaaatgatattcatctgttaatttatcattgccttgatgttgctgctgttgcagattgctggtgggatcaatcagtcgtactgcaaaatactttttgccgaaatgaaatgctatcaaaacagagggtgaaggcctggctgttatttttcattgctcttcatgatattggaaagtttgatatacgattccaatataaatcagcagaaagttggctgaaattaaatcctgcaacgccatcacttaatggtccatcaacacaaatgtgccgtaaatttaatcatggtgcagccggtctgtattggtttaaccaggattcactttcagagcaatctctcggggattttttcagtttttttgatgccgctcctcatccttatgagtcctggtttccatgggtagaggccgttacaggacatcatggttttatattacattcccaggatcaagataagtcgcgttgggaaatgccagcttctctggcatcttatgctgcgcaagataaacaggctcgtgaggagtggatatctgtactggaagcattatttttaacgccagcggggttatctataaacgatataccacctgattgttcatcactgttagcaggtttttgctcgcttgctgactggttaggctcctggactacaacgaatacctttctgtttaatgaggatgcgccttccgacataaatgctctgagaacgtatttccaggaccgacagcaggatgcgagccgggtattggagttgagtggacttgtatcaaataagcgatgttatgaaggtgttcatgcactactggacaatggctatcaacccagacaattacaggtgttagttgatgctcttccagtagctcccgggctgacggtaatagaggcacctacaggctccggtaaaacggaaacagcgctggcctatgcttggaaacttattgatcaacaaattgcggatagtgttatttttgccctcccaacacaagctaccgcgaatgctatgcttacgagaatggaagcgagcgcgagccacttattttcatccccaaatcttattcttgctcatggcaattcacggtttaaccacctctttcaatcaataaaatcacgcgcgattactgaacaggggcaagaagaagcgtgggttcagtgttgtcagtggttgtcacaaagcaataagaaagtgtttcttgggcaaatcggcgtttgcacgattgatcaggtgttgatatcggtattgccagttaaacaccgctttatccgtggtttgggaattggtcgaagtgttttaattgttgatgaagttcatgcttacgacacctatatgaacggcttgctggaggcagtgctcaaggctcaggctgatgtgggagggagtgttattcttctttccgcaaccctaccaatgaaacaaaaacagaaacttctggatacttatggtctgcatacagatccagtggaaaataactccgcatatccactcattaactggcgaggtgtgaatggtgcgcaacgttttgatctgctagctcatccagaacaactcccgccccgcttttcgattcagccagaacctatttgtttagctgacatgttacctgaccttacgatgttagagcgaatgatcgcagcggcaaacgcgggtgcacaggtctgtcttatttgcaatttggttgacgttgcacaagtatgctaccaacggctaaaggagctaaataacacgcaagtagatatagatttgtttcatgcgcgctttacgctgaacgatcgtcgtgaaaaagagaatcgagttattagcaatttcggcaaaaatgggaagcgaaatgttggacggatacttgtcgcaacccaggtcgtggaacaatcactcgacgttgattttgattggttaattactcagcattgtcctgcagatttgcttttccaacgattgggccgtttacatcgccatcatcgcaaatatcgtcccgctggttttgagattcctgttgccaccattttgctgcctgatggcgagggttacggacgacatgagcatatttatagcaacgttagagtcatgtggcggacgcagcaacatattgaggagcttaatggagcatccttatttttccctgatgcttaccggcaatggctggatagcatttacgatgatgcggaaatggatgagccagaatgggtcggcaatggcatggataaatttgaaagcgccgagtgtgaaaaaaggttcaaggctcgcaaggtcctgcagtgggctgaagaatatagcttgcaggataacgatgaaaccattcttgcggtaacgagggatggggaaatgagcctgccattattgccttatgtacaaacgtcttcaggtaaacaactgctcgatggccaggtctacgaggacctaagtcatgaacagcagtatgaggcgcttgcacttaatcgcgtcaatgtacccttcacctggaaacgtagtttttctgaagtagtagatgaagatgggttactttggctggaagggaaacagaatctggatggatgggtctggcagggtaacagtattgttattacctatacaggggatgaagggatgaccagagtcatccctgcaaatcccaaa

SEQ ID NO: 2 - DNA, 1527 bp, synthetic construct
  misc_feature 1..1527: note = Synthetic sequence, modified Cse1 with NLS
  source 1..1527: mol_type = other DNA
atgcccaagaagaagcggaaggtgaacctgctgattgacaactggatccctgtgcgcccacggaacggaggaaaagtccagattattaatctgcagagcctgtactgctcccgggatcagtggagactgagcctgcccagagacgatatggagctggccgccctggccctgctggtgtgcatcggccagatcatcgcccctgccaaggacgatgtggagttcaggcaccgcatcatgaaccctctgaccgaggatgagtttcagcagctgatcgccccatggatcgacatgttctatctgaatcacgccgagcaccccttcatgcagacaaagggcgtgaaggccaacgacgtgacccccatggagaagctgctggcaggcgtgtccggagcaacaaattgcgccttcgtgaaccagccaggacagggagaggccctgtgcggaggctgtaccgccatcgccctgtttaatcaggcaaaccaggcacctggattcggaggaggctttaagtctggactgaggggaggaaccccagtgaccacattcgtgagaggcatcgatctgaggagcacagtgctgctgaatgtgctgaccctgccacggctgcagaagcagtttcccaatgagagccacacagagaaccagcccacctggatcaagcctatcaagtctaacgagagcatccctgccagctccatcggcttcgtgagaggcctgttttggcagccagcccacatcgagctgtgcgaccccatcggcatcggcaagtgttcttgctgtggccaggaaagcaatctgaggtacaccggcttcctgaaggagaagttcacctttacagtgaacggcctgtggccccaccctcactctccatgtctggtgacagtgaagaagggcgaggtggaggagaagttcctggcctttaccacatccgccccctcttggacccagatcagcagagtggtggtggacaagatcatccagaacgagaatggcaacagagtggccgccgtggtgaatcagttcaggaacatcgccccacagtctcccctggagctgatcatgggcggctacaggaacaatcaggccagcatcctggagcggagacacgatgtgctgatgtttaatcagggctggcagcagtatggcaatgtgatcaacgagatcgtgacagtgggcctgggctacaagaccgccctgagaaaggccctgtatacattcgccgagggctttaagaacaaggacttcaagggagcaggcgtgagcgtgcacgagaccgccgagaggcacttttaccgccagtccgagctgctgatccccgatgtgctggccaatgtgaacttctcccaggccgacgaagtgatcgccgatctgagggacaagctgcaccagctgtgcgagatgctgtttaaccagtctgtggccccatacgcccaccaccccaagctgatcagcacactggccctggcaagggccaccctgtataagcacctgagggagctgaagccacagggaggaccttctaatgga

SEQ ID NO: 3 - DNA, 501 bp, synthetic construct
  misc_feature 1..501: note = Synthetic sequence, modified Cse2 with NLS
  source 1..501: mol_type = other DNA
atgcccaagaagaagcggaaggtggccgatgagatcgacgcaatggcactgtacagggcatggcagcagctggacaacggatcttgcgcacagatcaggcgcgtgagcgagcctgatgagctgagggacatcccagccttctatcggctggtgcagccctttggctgggagaatcctagacaccagcaggccctgctgaggatggtgttttgtctgagcgccggcaagaacgtgatccggcaccaggacaagaagagcgagcagaccacaggaatctccctgggacgcgccctggccaattccggccggatcaacgagcggagaatcttccagctgatcagggccgatcgcacagccgacatggtgcagctgaggcgcctgctgacccacgcagagcctgtgctggattggccactgatggcccgcatgctgacatggtggggcaagcgggagagacagcagctgctggaggacttcgtgctgaccacaaataagaacgcc

SEQ ID NO: 4 - DNA, 693 bp, synthetic construct
  misc_feature 1..693: note = Synthetic sequence, modified Cas5 with NLS
  source 1..693: mol_type = other DNA
atgcccaagaagaagcggaaggtgcgctcctacctgatcctgagactggcaggaccaatgcaggcatggggacagcctacattcgagggaaccaggccaacaggccgctttcctacccggtctggactgctgggactgctgggagcctgcctgggcatccagagggacgatacctctagcctgcaggccctgagcgagtccgtgcagttcgccgtgcgctgtgatgagctgatcctggacgataggcgcgtgtccgtgacaggcctgcgggattaccacaccgtgctgggcgccagagaggactataggggcctgaagtcccacgagaccatccagacatggcgcgagtacctgtgcgacgcctcttttacagtggccctgtggctgaccccacacgcaacaatggtcatcagcgagctggagaaggccgtgctgaagccacggtacaccccctatctgggccggagaagctgccctctgacacacccactgttcctgggcacctgtcaggcctccgatccccagaaggccctgctgaactacgagcctgtgggcggcgacatctattctgaggagagcgtgacaggccaccacctgaagttcaccgccagggatgagccaatgatcacactgccaaggcagtttgcatccagggagtggtatgtgatcaagggaggaatggacgtgagccag

SEQ ID NO: 5 - DNA, 618 bp, synthetic construct
  misc_feature 1..618: note = Synthetic sequence, modified Cas6 with NLS
  source 1..618: mol_type = other DNA
atgcccaagaagaagcggaaggtgtacctgagcaaagtgatcatcgcaagggcatggtccagggacctgtatcagctgcaccagggcctgtggcacctgttccctaatagaccagatgccgccagggacttcctgtttcacgtggagaagaggaacacacccgagggctgtcacgtgctgctgcagtccgcccagatgcccgtgagcaccgcagtggccacagtgatcaagaccaagcaggtggagttccagctgcaagtgggcgtgccactgtactttaggctgcgcgccaatcccatcaagaccatcctggataaccagaagcgcctggactctaagggcaatatcaagcggtgcagagtgcctctgatcaaggaggccgagcagatcgcctggctgcagagaaagctgggcaacgccgccagggtggaggatgtgcaccctatcagcgagcggccacagtatttcagcggcgacggcaagtccggcaagatccagaccgtgtgctttgagggcgtgctgaccatcaacgatgccccagccctgatcgacctggtgcagcagggaatcggacctgctaagtcaatgggatgtgggctgctgtcactggcacctctg

SEQ ID NO: 6 - DNA, 1110 bp, synthetic construct
  misc_feature 1..1110: note = Synthetic sequence, modified Cas7 with NLS
  source 1..1110: mol_type = other DNA
atgcccaagaagaagcggaaggtgtccaatttcatcaacatccacgtgctgatctcccactctccaagctgcctgaatagagacgatatgaacatgcagaaggacgccatctttggcggcaagcggagagtgaggatctctagccagtccctgaagcgggccatgagaaagtctggctactatgcccagaatatcggcgagtcctctctgaggaccatccacctggcacagctgagggacgtgctgagacagaagctgggcgagcggtttgatcagaagatcatcgacaagacactggccctgctgagcggcaagtccgtggatgaggccgagaagatcagcgccgacgcagtgaccccatgggtggtgggagagatcgcatggttctgtgagcaggtggccaaggccgaggccgataatctggacgataagaagctgctgaaggtgctgaaggaggatatcgccgccatcagagtgaacctgcagcagggagtggacatcgccctgagcggcaggatggccacatccggcatgatgaccgagctgggcaaggtggacggagcaatgtccatcgcacacgccatcaccacacaccaggtggactctgatatcgactggttcacagccgtggacgatctgcaggagcagggaagcgcccacctgggaacccaggagttcagctccggcgtgttttacagatatgccaatatcaacctggcacagctgcaggagaacctgggaggagcatccagggagcaggccctggagatcgccacacacgtggtgcacatgctggcaaccgaggtgccaggagcaaagcagcgcacctacgccgccttcaatcctgccgatatggtcatggtgaacttttccgacatgccactgtctatggccaatgccttcgagaaggccgtgaaggccaaggatggcttcctgcagccttccatccaggcctttaaccagtactgggaccgcgtggccaatggatatggcctgaacggagctgccgcccagttttccctgtctgatgtggaccctatcacagcccaggtgaagcagatgccaaccctggagcagctgaagagctgggtgcggaacaatggagaggca

SEQ ID NO: 7 - DNA, 2775 bp, synthetic construct
  misc_feature 1..2775: note = Synthetic sequence, modified Cas3 with BPNLS
  source 1..2775: mol_type = other DNA
aagcggactgctgatggcagtgaatttgagtccccaaagaagaagagaaaggtggaagaaccttttaaatatatatgccattactggggaaaatcctcaaaaagcttgacgaaaggaaatgatattcatctgttaatttatcattgccttgatgttgctgctgttgcagattgctggtgggatcaatcagtcgtactgcaaaatactttttgccgaaatgaaatgctatcaaaacagagggtgaaggcctggctgttatttttcattgctcttcatgatattggaaagtttgatatacgattccaatataaatcagcagaaagttggctgaaattaaatcctgcaacgccatcacttaatggtccatcaacacaaatgtgccgtaaatttaatcatggtgcagccggtctgtattggtttaaccaggattcactttcagagcaatctctcggggattttttcagtttttttgatgccgctcctcatccttatgagtcctggtttccatgggtagaggccgttacaggacatcatggttttatattacattcccaggatcaagataagtcgcgttgggaaatgccagcttctctggcatcttatgctgcgcaagataaacaggctcgtgaggagtggatatctgtactggaagcattatttttaacgccagcggggttatctataaacgatataccacctgattgttcatcactgttagcaggtttttgctcgcttgctgactggttaggctcctggactacaacgaatacctttctgtttaatgaggatgcgccttccgacataaatgctctgagaacgtatttccaggaccgacagcaggatgcgagccgggtattggagttgagtggacttgtatcaaataagcgatgttatgaaggtgttcatgcactactggacaatggctatcaacccagacaattacaggtgttagttgatgctcttccagtagctcccgggctgacggtaatagaggcacctacaggctccggtaaaacggaaacagcgctggcctatgcttggaaacttattgatcaacaaattgcggatagtgttatttttgccctcccaacacaagctaccgcgaatgctatgcttacgagaatggaagcgagcgcgagccacttattttcatccccaaatcttattcttgctcatggcaattcacggtttaaccacctctttcaatcaataaaatcacgcgcgattactgaacaggggcaagaagaagcgtgggttcagtgttgtcagtggttgtcacaaagcaataagaaagtgtttcttgggcaaatcggcgtttgcacgattgatcaggtgttgatatcggtattgccagttaaacaccgctttatccgtggtttgggaattggtcgaagtgttttaattgttgatgaagttcatgcttacgacacctatatgaacggcttgctggaggcagtgctcaaggctcaggctgatgtgggagggagtgttattcttctttccgcaaccctaccaatgaaacaaaaacagaaacttctggatacttatggtctgcatacagatccagtggaaaataactccgcatatccactcattaactggcgaggtgtgaatggtgcgcaacgttttgatctgctagctcatccagaacaactcccgccccgcttttcgattcagccagaacctatttgtttagctgacatgttacctgaccttacgatgttagagcgaatgatcgcagcggcaaacgcgggtgcacaggtctgtcttatttgcaatttggttgacgttgcacaagtatgctaccaacggctaaaggagctaaataacacgcaagtagatatagatttgtttcatgcgcgctttacgctgaacgatcgtcgtgaaaaagagaatcgagttattagcaatttcggcaaaaatgggaagcgaaatgttggacggatacttgtcgcaacccaggtcgtggaacaatcactcgacgttgattttgattggttaattactcagcattgtcctgcagatttgcttttccaacgattgggccgtttacatcgccatcatcgcaaatatcgtcccgctggttttgagattcctgttgccaccattttgctgcctgatggcgagggttacggacgacatgagcatatttatagcaacgttagagtcatgtggcggacgcagcaacatattgaggagcttaatggagcatccttatttttccctgatgcttaccggcaatggctggatagcatttacgatgatgcggaaatggatgagccagaatgggtcggcaatggcatggataaatttgaaagcgccgagtgtgaaaaaaggttcaaggctcgcaaggtcctgcagtgggctgaagaatatagcttgcaggataacgatgaaaccattcttgcggtaacgagggatggggaaatgagcctgccattattgccttatgtacaaacgtcttcaggtaaacaactgctcgatggccaggtctacgaggacctaagtcatgaacagcagtatgaggcgcttgcacttaatcgcgtcaatgtacccttcacctggaaacgtagtttttctgaagtagtagatgaagatgggttactttggctggaagggaaacagaatctggatggatgggtctggcagggtaacagtattgttattacctatacaggggatgaagggatgaccagagtcatccctgcaaatcccaaaaagcggactgctgatggcagtgaatttgagtccccaaagaagaagagaaaggtggaa

SEQ ID NO: 8 - DNA, 1617 bp, synthetic construct
  misc_feature 1..1617: note = Synthetic sequence, modified Cse1 with BPNLS
  source 1..1617: mol_type = other DNA
aagcggactgctgatggcagtgaatttgagtccccaaagaagaagagaaaggtggaaaacctgctgattgacaactggatccctgtgcgcccacggaacggaggaaaagtccagattattaatctgcagagcctgtactgctcccgggatcagtggagactgagcctgcccagagacgatatggagctggccgccctggccctgctggtgtgcatcggccagatcatcgcccctgccaaggacgatgtggagttcaggcaccgcatcatgaaccctctgaccgaggatgagtttcagcagctgatcgccccatggatcgacatgttctatctgaatcacgccgagcaccccttcatgcagacaaagggcgtgaaggccaacgacgtgacccccatggagaagctgctggcaggcgtgtccggagcaacaaattgcgccttcgtgaaccagccaggacagggagaggccctgtgcggaggctgtaccgccatcgccctgtttaatcaggcaaaccaggcacctggattcggaggaggctttaagtctggactgaggggaggaaccccagtgaccacattcgtgagaggcatcgatctgaggagcacagtgctgctgaatgtgctgaccctgccacggctgcagaagcagtttcccaatgagagccacacagagaaccagcccacctggatcaagcctatcaagtctaacgagagcatccctgccagctccatcggcttcgtgagaggcctgttttggcagccagcccacatcgagctgtgcgaccccatcggcatcggcaagtgttcttgctgtggccaggaaagcaatctgaggtacaccggcttcctgaaggagaagttcacctttacagtgaacggcctgtggccccaccctcactctccatgtctggtgacagtgaagaagggcgaggtggaggagaagttcctggcctttaccacatccgccccctcttggacccagatcagcagagtggtggtggacaagatcatccagaacgagaatggcaacagagtggccgccgtggtgaatcagttcaggaacatcgccccacagtctcccctggagctgatcatgggcggctacaggaacaatcaggccagcatcctggagcggagacacgatgtgctgatgtttaatcagggctggcagcagtatggcaatgtgatcaacgagatcgtgacagtgggcctgggctacaagaccgccctgagaaaggccctgtatacattcgccgagggctttaagaacaaggacttcaagggagcaggcgtgagcgtgcacgagaccgccgagaggcacttttaccgccagtccgagctgctgatccccgatgtgctggccaatgtgaacttctcccaggccgacgaagtgatcgccgatctgagggacaagctgcaccagctgtgcgagatgctgtttaaccagtctgtggccccatacgcccaccaccccaagctgatcagcacactggccctggcaagggccaccctgtataagcacctgagggagctgaagccacagggaggaccttctaatggaaagcggactgctgatggcagtgaatttgagtccccaaagaagaagagaaaggtggaa

SEQ ID NO: 9 - DNA, 591 bp, synthetic construct
  misc_feature 1..591: note = Synthetic sequence, modified Cse2 with BPNLS
  source 1..591: mol_type = other DNA
aagcggactgctgatggcagtgaatttgagtccccaaagaagaagagaaaggtggaagccgatgagatcgacgcaatggcactgtacagggcatggcagcagctggacaacggatcttgcgcacagatcaggcgcgtgagcgagcctgatgagctgagggacatcccagccttctatcggctggtgcagccctttggctgggagaatcctagacaccagcaggccctgctgaggatggtgttttgtctgagcgccggcaagaacgtgatccggcaccaggacaagaagagcgagcagaccacaggaatctccctgggacgcgccctggccaattccggccggatcaacgagcggagaatcttccagctgatcagggccgatcgcacagccgacatggtgcagctgaggcgcctgctgacccacgcagagcctgtgctggattggccactgatggcccgcatgctgacatggtggggcaagcgggagagacagcagctgctggaggacttcgtgctgaccacaaataagaacgccaagcggactgctgatggcagtgaatttgagtccccaaagaagaagagaaaggtggaa

SEQ ID NO: 10 - DNA, 783 bp, synthetic construct
  misc_feature 1..783: note = Synthetic sequence, modified Cas5 with BPNLS
  source 1..783: mol_type = other DNA
aagcggactgctgatggcagtgaatttgagtccccaaagaagaagagaaaggtggaacgctcctacctgatcctgagactggcaggaccaatgcaggcatggggacagcctacattcgagggaaccaggccaacaggccgctttcctacccggtctggactgctgggactgctgggagcctgcctgggcatccagagggacgatacctctagcctgcaggccctgagcgagtccgtgcagttcgccgtgcgctgtgatgagctgatcctggacgataggcgcgtgtccgtgacaggcctgcgggattaccacaccgtgctgggcgccagagaggactataggggcctgaagtcccacgagaccatccagacatggcgcgagtacctgtgcgacgcctcttttacagtggccctgtggctgaccccacacgcaacaatggtcatcagcgagctggagaaggccgtgctgaagccacggtacaccccctatctgggccggagaagctgccctctgacacacccactgttcctgggcacctgtcaggcctccgatccccagaaggccctgctgaactacgagcctgtgggcggcgacatctattctgaggagagcgtgacaggccaccacctgaagttcaccgccagggatgagccaatgatcacactgccaaggcagtttgcatccagggagtggtatgtgatcaagggaggaatggacgtgagccagaagcggactgctgatggcagtgaatttgagtccccaaagaagaagagaaaggtggaa

SEQ ID NO: 11 - DNA, 708 bp, synthetic construct
  misc_feature 1..708: note = Synthetic sequence, modified Cas6 with BPNLS
  source 1..708: mol_type = other DNA
aagcggactgctgatggcagtgaatttgagtccccaaagaagaagagaaaggtggaatacctgagcaaagtgatcatcgcaagggcatggtccagggacctgtatcagctgcaccagggcctgtggcacctgttccctaatagaccagatgccgccagggacttcctgtttcacgtggagaagaggaacacacccgagggctgtcacgtgctgctgcagtccgcccagatgcccgtgagcaccgcagtggccacagtgatcaagaccaagcaggtggagttccagctgcaagtgggcgtgccactgtactttaggctgcgcgccaatcccatcaagaccatcctggataaccagaagcgcctggactctaagggcaatatcaagcggtgcagagtgcctctgatcaaggaggccgagcagatcgcctggctgcagagaaagctgggcaacgccgccagggtggaggatgtgcaccctatcagcgagcggccacagtatttcagcggcgacggcaagtccggcaagatccagaccgtgtgctttgagggcgtgctgaccatcaacgatgccccagccctgatcgacctggtgcagcagggaatcggacctgctaagtcaatgggatgtgggctgctgtcactggcacctctgaagcggactgctgatggcagtgaatttgagtccccaaagaagaagagaaaggtggaa

SEQ ID NO: 12 - DNA, 1200 bp, synthetic construct
  misc_feature 1..1200: note = Synthetic sequence, modified Cas7 with BPNLS
  source 1..1200: mol_type = other DNA
aagcggactgctgatggcagtgaatttgagtccccaaagaagaagagaaaggtggaatccaatttcatcaacatccacgtgctgatctcccactctccaagctgcctgaatagagacgatatgaacatgcagaaggacgccatctttggcggcaagcggagagtgaggatctctagccagtccctgaagcgggccatgagaaagtctggctactatgcccagaatatcggcgagtcctctctgaggaccatccacctggcacagctgagggacgtgctgagacagaagctgggcgagcggtttgatcagaagatcatcgacaagacactggccctgctgagcggcaagtccgtggatgaggccgagaagatcagcgccgacgcagtgaccccatgggtggtgggagagatcgcatggttctgtgagcaggtggccaaggccgaggccgataatctggacgataagaagctgctgaaggtgctgaaggaggatatcgccgccatcagagtgaacctgcagcagggagtggacatcgccctgagcggcaggatggccacatccggcatgatgaccgagctgggcaaggtggacggagcaatgtccatcgcacacgccatcaccacacaccaggtggactctgatatcgactggttcacagccgtggacgatctgcaggagcagggaagcgcccacctgggaacccaggagttcagctccggcgtgttttacagatatgccaatatcaacctggcacagctgcaggagaacctgggaggagcatccagggagcaggccctggagatcgccacacacgtggtgcacatgctggcaaccgaggtgccaggagcaaagcagcgcacctacgccgccttcaatcctgccgatatggtcatggtgaacttttccgacatgccactgtctatggccaatgccttcgagaaggccgtgaaggccaaggatggcttcctgcagccttccatccaggcctttaaccagtactgggaccgcgtggccaatggatatggcctgaacggagctgccgcccagttttccctgtctgatgtggaccctatcacagcccaggtgaagcagatgccaaccctggagcagctgaagagctgggtgcggaacaatggagaggcaaagcggactgctgatggcagtgaatttgagtccccaaagaagaagagaaaggtggaa

SEQ ID NO: 13 - DNA, 2664 bp, Escherichia coli
  source 1..2664: mol_type = unassigned DNA
atggagccattcaaatacatctgccactactgggggaaatcatctaaaagcctgacaaaggggaacgatatccacctgctgatctaccactgcctggacgtggcagcagtggcagattgttggtgggaccagagcgtggtgctgcagaataccttctgccggaacgagatgctgtccaagcagagagtgaaggcctggctgctgttctttatcgccctgcacgatatcggcaagttcgacatcaggtttcagtataagtctgccgagagctggctgaagctgaatccagccacaccctccctgaacggcccttctacccagatgtgcaggaagttcaatcacggagcagcaggactgtactggtttaaccaggacagcctgtccgagcagtctctgggcgatttctttagcttctttgacgccgcccctcacccatatgagagctggttcccatgggtggaggcagtgacaggacaccacggctttatcctgcactcccaggaccaggataagtctagatgggagatgccagcatccctggcatcttacgcagcacaggataagcaggcaagggaggagtggatctctgtgctggaggccctgttcctgaccccagcaggcctgagcatcaatgatatcccacctgactgcagctccctgctggcaggcttttgtagcctggcagactggctgggatcctggaccacaaccaatacattcctgtttaacgaggatgccccttctgacatcaacgccctgcgcacctacttccaggatcggcagcaggacgccagcagagtgctggagctgtctggcctggtgagcaataagcggtgctacgagggagtgcacgcactgctggataacggctatcagcctagacagctgcaggtgctggtggacgcactgcctgtggcaccaggactgacagtgatcgaggcaccaaccggctctggcaagacagagaccgccctggcctatgcctggaagctgatcgatcagcagatcgccgacagcgtgatcttcgcactgccaacacaggcaaccgcaaatgccatgctgaccaggatggaggcctctgccagccacctgttttctagccctaacctgatcctggcccacggcaacagccggttcaatcacctgtttcagagcatcaagtccagagccatcacagagcagggacaggaggaggcatgggtgcagtgctgtcagtggctgtcccagtctaacaagaaggtgttcctgggccagatcggcgtgtgcaccatcgatcaggtgctgatctccgtgctgccagtgaagcacaggtttatcaggggactgggaatcggccgctctgtgctgatcgtggatgaggtgcacgcctacgacacatatatgaacggcctgctggaggccgtgctgaaggcacaggcagacgtgggaggaagcgtgatcctgctgtccgccaccctgcccatgaagcagaagcagaagctgctggatacatacggcctgcacaccgaccctgtggagaacaatagcgcctatccactgatcaattggaggggagtgaacggagcacagcggttcgacctgctggcacacccagagcagctgccaccacggttttccatccagcccgagcctatctgcctggccgatatgctgcccgacctgaccatgctggagagaatgatcgctgccgccaatgcaggagcacaggtgtgcctgatctgtaacctggtggatgtggcccaggtgtgctaccagcggctgaaggagctgaacaatacacaggtggacatcgatctgttccacgccaggtttaccctgaatgaccggagagagaaggagaaccgcgtgatctccaacttcggcaagaatggcaagagaaacgtgggcaggatcctggtggccacacaggtggtggagcagtctctggacgtggatttcgactggctgatcacccagcactgccctgccgatctgctgtttcagcggctgggcagactgcacagacaccacaggaagtacaggccagcaggatttgagatcccagtggccacaatcctgctgccagacggagagggatacggccggcacgagcacatctatagcaatgtgcgcgtgatgtggcggacccagcagcacatcgaggagctgaacggcgcctccctgttctttcccgatgcctacagacagtggctggactctatctatgacgatgccgagatggatgagcctgagtgggtgggcaatggcatggacaagttcgagtccgccgagtgtgagaagcggttcaaggccaggaaggtgctgcagtgggccgaggagtacagcctgcaggataacgacgagacaatcctggccgtgaccagggatggcgagatgtccctgcccctgctgccttatgtgcagacatcctctggcaagcagctgctggatggccaggtgtacgaggacctgagccacgagcagcagtatgaggccctggccctgaacagggtgaatgtgcccttcacctggaagcgcagcttttccgaagtggtggatgaggacggcctgctgtggctggagggcaagcagaatctggacggctgggtgtggcagggcaactccatcgtgattacctacaccggagacgaagggatgacaagagtgattcctgctaacccaaag

SEQ ID NO: 14 - DNA, 1506 bp, Escherichia coli
  source 1..1506: mol_type = unassigned DNA
atgaatttgcttattgataactggatccctgtacgcccgcgaaacggggggaaagtccaaatcataaatctgcaatcgctatactgcagtagagatcagtggcgattaagtttgccccgtgacgatatggaactggccgctttagcactgctggtttgcattgggcaaattatcgccccggcaaaagatgacgttgaatttcgacatcgcataatgaatccgctcactgaagatgagtttcaacaactcatcgcgccgtggatagatatgttctaccttaatcacgcagaacatccctttatgcagaccaaaggtgtcaaagcaaatgatgtgactccaatggaaaaactgttggctggggtaagcggcgcgacgaattgtgcatttgtcaatcaaccggggcagggtgaagcattatgtggtggatgcactgcgattgcgttattcaaccaggcgaatcaggcaccaggttttggtggtggttttaaaagcggtttacgtggaggaacacctgtaacaacgttcgtacgtgggatcgatcttcgttcaacggtgttactcaatgtcctcacattacctcgtcttcaaaaacaatttcctaatgaatcacatacggaaaaccaacctacctggattaaacctatcaagtccaatgagtctatacctgcttcgtcaattgggtttgtccgtggtctattctggcaaccagcgcatattgaattatgcgatcccattgggattggtaaatgttcttgctgtggacaggaaagcaatttgcgttataccggttttcttaaggaaaaatttacctttacagttaatgggctatggccccatccgcattccccttgtctggtaacagtcaagaaaggggaggttgaggaaaaatttcttgctttcaccacctccgcaccatcatggacacaaatcagccgagttgtggtagataagattattcaaaatgaaaatggaaatcgcgtggcggcggttgtgaatcaattcagaaatattgcgccgcaaagtcctcttgaattgattatggggggatatcgtaataatcaagcatctattcttgaacggcgtcatgatgtgttgatgtttaatcaggggtggcaacaatacggcaatgtgataaacgaaatagtgactgttggtttgggatataaaacagccttacgcaaggcgttatatacctttgcagaagggtttaaaaataaagacttcaaaggggccggagtctctgttcatgagactgcagaaaggcatttctatcgacagagtgaattattaattcccgatgtactggcgaatgttaatttttcccaggctgatgaggtaatagctgatttacgagacaaacttcatcaattgtgtgaaatgctatttaatcaatctgtagctccctatgcacatcatcctaaattaataagcacattagcgcttgcccgcgccacgctatacaaacatttacgggagttaaaaccgcaaggagggccatcaaatggc

SEQ ID NO: 15 - DNA, 480 bp, Escherichia coli
  source 1..480: mol_type = unassigned DNA
atggctgatgaaattgatgcaatggctttatatcgagcctggcaacaactggataatggatcatgtgcgcaaattagacgtgtttcagaacctgatgaattacgcgatatccctgcgttttataggctggtgcaaccttttggttgggaaaacccacgtcaccagcaggctcttttgcgcatggtgttttgcctgagcgcaggaaagaatgtcatccgacatcaggacaaaaaatcggagcaaacaacaggtatctcgttgggaagagctttagccaatagtggaagaattaacgagcgccgtatctttcaattaattcgggctgacagaacagccgatatggtccagttacgtcgattacttactcacgccgaacccgtacttgactggccattaatggccaggatgttgacctggtggggaaagcgcgaacgccagcaacttctggaagattttgtattgaccacaaacaaaaatgcg

SEQ ID NO: 16 - DNA, 672 bp, Escherichia coli
  source 1..672: mol_type = unassigned DNA
atgagatcttatttgatcttgcggcttgctgggccaatgcaagcctgggggcagccgacctttgaaggaacgcgacctaccggaagatttccgacccgaagcgggttattagggctactcggggcttgtcttgggatccaacgtgatgatacttcttcattacaggcgttatcagagagtgtgcaatttgcagtgcgctgcgatgaactcattcttgacgatcgtcgtgtgtctgtaacggggttgcgtgattaccatacagtccttggagcgcgagaagattaccgtggtttgaaaagtcatgaaacgattcaaacatggcgcgaatatttatgtgatgcctcctttaccgtcgctctctggttaacaccccatgcaacgatggttatctcagaacttgaaaaagcagtattaaagcctcggtatacaccttacctggggcggagaagttgcccactaacacacccgctttttttggggacatgtcaggcatcggatcctcagaaggcgctattaaattatgagcccgttggcggcgatatatatagtgaggaatcagttacagggcatcatttaaaatttacggcgcgcgacgaaccgatgatcaccttgcctcgacaatttgcttcccgagaatggtatgtgattaaaggaggtatggatgtatctcag

SEQ ID NO: 17 - DNA, 597 bp, Escherichia coli
  source 1..597: mol_type = unassigned DNA
atgtatctcagtaaagtcatcattgccagggcctggagcagggatctttaccaacttcaccagggattatggcatttatttccaaacagaccggatgctgctcgtgattttctttttcatgttgagaagcgaaacacaccagaaggctgtcatgttttattgcagtcagcgcaaatgcctgtttcaactgccgttgcgacagtcattaaaactaaacaggttgaatttcaacttcaggttggtgttccactctattttcggcttcgggcaaatccgatcaaaactattctcgacaatcaaaagcgcctggacagtaaagggaatattaaacgctgtcgggttccgttaataaaagaagcagaacaaatcgcgtggttgcaacgtaaattgggcaatgcggcgcgcgttgaagatgtgcatcccatatcggaacggccacagtatttttctggtgatggtaaaagtggaaagatccaaacggtttgctttgaaggtgtgctcaccatcaacgacgcgccagcgttaatagatcttgtacagcaaggtattgggccagctaaatcgatgggatgtggcttgctatctttggctccactg

SEQ ID NO: 18 - DNA, 1086 bp, Escherichia coli
  source 1..1086: mol_type = unassigned DNA
tctaactttatcaatattcatgttctgatctctcacagcccttcatgtctgaaccgcgacgatatgaacatgcagaaagacgctattttcggcggcaaaagacgagtaagaatttcaagtcaaagccttaaacgtgcgatgcgtaaaagtggttattacgcacaaaatattggtgaatccagtctcagaaccattcatcttgcacaattacgtgatgttcttcggcaaaaacttggtgaacgttttgaccaaaaaatcatcgataagacattagcgctgctctccggtaaatcagttgatgaagccgaaaagatttctgccgatgcggttactccctgggttgtgggagaaatagcctggttctgtgagcaggttgcaaaagcagaggctgataatctggatgataaaaagctgctcaaagttcttaaggaagatattgccgccatacgtgtgaatttacagcagggtgttgatattgcgcttagtggaagaatggcaaccagcggcatgatgactgagttgggaaaagttgatggtgcaatgtccattgcgcatgcgatcactactcatcaggttgattctgatattgactggttcaccgctgtagatgatttacaggaacaaggttctgcacatctgggaactcaggaattttcatcgggtgttttttatcgttatgccaacattaacctcgctcaacttcaggaaaatttaggtggtgcctccagggagcaggctctggaaattgcaacccatgttgttcatatgctggcaacagaggtccctggagcaaaacagcgtacttatgccgcttttaaccctgcggatatggtaatggttaatttctccgatatgccactttctatggcaaatgcttttgaaaaagcggttaaagcgaaagatggctttttgcaaccgtctatacaggcgtttaatcaatattgggatcgcgttgccaatggatatggtctgaacggagctgctgcgcaattcagcttatctgatgtagacccaattactgctcaagttaaacaaatgcctactttagaacagttaaaatcctgggttcgtaataatggcgaggcg

SEQ ID NO: 19 - DNA, 39 bp, synthetic construct
  misc_feature 1..39: note = Synthetic sequence, target (CCR5)
  source 1..39: mol_type = other DNA
tcaagtccaatctatgacatcaattattatacatcggag

SEQ ID NO: 20 - DNA, 50 bp, synthetic construct
  misc_feature 1..50: note = Synthetic sequence, reporter vector insert1 (CCR5)
  source 1..50: mol_type = other DNA
gtcggattcaagtccaatctatgacatcaattattatacatcggagaggt

SEQ ID NO: 21 - DNA, 50 bp, synthetic construct
  misc_feature 1..50: note = Synthetic sequence, reporter vector insert2 (CCR5)
  source 1..50: mol_type = other DNA
cggtacctctccgatgtataataattgatgtcatagattggacttgaatc

SEQ ID NO: 22 - DNA, 35 bp, synthetic construct
  misc_feature 1..35: note = Synthetic sequence, target (E. coli)
  source 1..35: mol_type = other DNA
aagagcacaaatatcatcgctcaaaccacttacgg

SEQ ID NO: 23 - DNA, 46 bp, synthetic construct
  misc_feature 1..46: note = Synthetic sequence, reporter vector insert1 (E. coli)
  source 1..46: mol_type = other DNA
gtcggataagagcacaaatatcatcgctcaaaccacttacggaggt

SEQ ID NO: 24 - DNA, 46 bp, synthetic construct
  misc_feature 1..46: note = Synthetic sequence, reporter vector insert2 (E. coli)
  source 1..46: mol_type = other DNA
cggtacctccgtaagtggtttgagcgatgatatttgtgctcttatc

SEQ ID NO: 25 - DNA, 36 bp, synthetic construct
  misc_feature 1..36: note = Synthetic sequence, crRNA insert1 (CCR5)
  source 1..36: mol_type = other DNA
accgtccaatctatgacatcaattattatacatcgg

SEQ ID NO: 26 - DNA, 36 bp, synthetic construct
  misc_feature 1..36: note = Synthetic sequence, crRNA insert 2(CCR5)
  source 1..36: mol_type = other DNA
acacccgatgtataataattgatgtcatagattgga

SEQ ID NO: 27 - DNA, 36 bp, synthetic construct
  misc_feature 1..36: note = Synthetic sequence, crRNA insert1 (E.coli)
  source 1..36: mol_type = other DNA
accgagcacaaatatcatcgctcaaaccacttacgg

SEQ ID NO: 28 - DNA, 36 bp, synthetic construct
  misc_feature 1..36: note = Synthetic sequence, crRNA insert 2(E.coli)
  source 1..36: mol_type = other DNA
acacccgtaagtggtttgagcgatgatatttgtgct

SEQ ID NO: 29 - DNA, 36 bp, synthetic construct
  misc_feature 1..36: note = Synthetic sequence, crRNA insert 1(EMX1)
  source 1..36: mol_type = other DNA
accgcaggccaatggggaggacatcgatgtcacctc

SEQ ID NO: 30 - DNA, 36 bp, synthetic construct
  misc_feature 1..36: note = Synthetic sequence, crRNA insert 2(EMX1)
  source 1..36: mol_type = other DNA
acacgaggtgacatcgatgtcctccccattggcctg

SEQ ID NO: 31 - DNA, 5701 bp, synthetic construct
  misc_feature 1..5701: note = Synthetic sequence, reporter vector (CCR5)
  source 1..5701: mol_type = other DNA
ggcctaactggccggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttggcaatccggtactgttggtaaagccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggactagggtctctgtcggattcaagtccaatctatgacatcaattattatacatcggagaggtaccgtgagacctaggagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgaattcttaactgcagtctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct

SEQ ID NO: 32 - DNA, 5697 bp, synthetic construct
  misc_feature 1..5697: note = Synthetic sequence, reporter vector (E. coli)
  source 1..5697: mol_type = other DNA
ggcctaactggccggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttggcaatccggtactgttggtaaagccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggactagggtctctgtcggataagagcacaaatatcatcgctcaaaccacttacggaggtaccgtgagacctaggagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgaattcttaactgcagtctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct

SEQ ID NO: 33 - DNA, 3391 bp, synthetic construct
  misc_feature 1..3391: note = Synthetic sequence, crRNA vector (CCR5)
  source 1..3391: mol_type = other DNA
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtaccgggccccccctcgaggtcgacggtatcgataagcttgatatcgaattgacgtgaattcttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgtggatgtgttgtttgtgtgatactataaagttggtagattgtgactggcttaaaaaatcattaattaataataggttatgtttagagtgttccccgcgccagcggggataaaccgtccaatctatgacatcaattattatacatcgggtgttccccgcgccagcggggataaaccgttttttgaattcctgcagcccgggggatccactagttctagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 34 - DNA, 3391 bp, synthetic construct
  misc_feature 1..3391: note = Synthetic sequence, crRNA vector (E. coli)
  source 1..3391: mol_type = other DNA
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtaccgggccccccctcgaggtcgacggtatcgataagcttgatatcgaattgacgtgaattcttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgtggatgtgttgtttgtgtgatactataaagttggtagattgtgactggcttaaaaaatcattaattaataataggttatgtttagagtgttccccgcgccagcggggataaaccgagcacaaatatcatcgctcaaaccacttacgggtgttccccgcgccagcggggataaaccgttttttgaattcctgcagcccgggggatccactagttctagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 35 - DNA, 3391 bp, synthetic construct
  misc_feature 1..3391: note = Synthetic sequence, crRNA vector (EMX1)
  source 1..3391: mol_type = other DNA
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtaccgggccccccctcgaggtcgacggtatcgataagcttgatatcgaattgacgtgaattcttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgtggatgtgttgtttgtgtgatactataaagttggtagattgtgactggcttaaaaaatcattaattaataataggttatgtttagagtgttccccgcgccagcggggataaaccgcaggccaatggggaggacatcgatgtcacctcgtgttccccgcgccagcggggataaaccgttttttgaattcctgcagcccgggggatccactagttctagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 36 - DNA, 61 bp, synthetic construct
  misc_feature 1..61: note = Synthetic sequence, primer1 (Cse1)
  source 1..61: mol_type = other DNA
gcaaagaattcagatctccaccatgcctaagaagaagagaaaagtgaacctgctgattgac

SEQ ID NO: 37 - DNA, 49 bp, synthetic construct
  misc_feature 1..49: note = Synthetic sequence, primer2 (Cse1)
  source 1..49: mol_type = other DNA
tcatcgatgcatctcgagttatccattagaaggtcctccctgtggcttc

SEQ ID NO: 38 - DNA, 61 bp, synthetic construct
  misc_feature 1..61: note = Synthetic sequence, primer1 (Cse2)
  source 1..61: mol_type = other DNA
gcaaagaattcagatctccaccatgcccaagaagaagcggaaggtggccgatgagatcgac

SEQ ID NO: 39 - DNA, 49 bp, synthetic construct
  misc_feature 1..49: note = Synthetic sequence, primer2 (Cse2)
  source 1..49: mol_type = other DNA
tcatcgatgcatctcgagttaggcgttcttatttgtggtcagcacgaag

SEQ ID NO: 40 - DNA, 61 bp, synthetic construct
  misc_feature 1..61: note = Synthetic sequence, primer1 (Cas5)
  source 1..61: mol_type = other DNA
gcaaagaattcagatctccaccatgcccaagaagaagcggaaggtgtccaatttcatcaac

SEQ ID NO: 41 - DNA, 49 bp, synthetic construct
  misc_feature 1..49: note = Synthetic sequence, primer2 (Cas5)
  source 1..49: mol_type = other DNA
tcatcgatgcatctcgagttatgcctctccattgttccgcacccagctc

SEQ ID NO: 42 - DNA, 61 bp, synthetic construct
  misc_feature 1..61: note = Synthetic sequence, primer1 (Cas6)
  source 1..61: mol_type = other DNA
gcaaagaattcagatctccaccatgcccaagaagaagcggaaagtgtacctgagcaaagtg

SEQ ID NO: 43 - DNA, 47 bp, synthetic construct
  misc_feature 1..47: note = Synthetic sequence, primer2 (Cas6)
  source 1..47: mol_type = other DNA
tcatcgatgcatctcgagttacagaggtgccagtgacagcagcccac

SEQ ID NO: 44 - DNA, 61 bp, synthetic construct
  misc_feature 1..61: note = Synthetic sequence, primer1 (Cas7)
  source 1..61: mol_type = other DNA
gcaaagaattcagatctccaccatgcccaagaagaagcggaaggtgcgctcctacctgatc

SEQ ID NO: 45 - DNA, 49 bp, synthetic construct
  misc_feature 1..49: note = Synthetic sequence, primer2 (Cas7)
  source 1..49: mol_type = other DNA
tcatcgatgcatctcgagttactggctcacgtccattcctcccttgatc

SEQ ID NO: 46 - DNA, 6265 bp, Homo sapiens
  source 1..6265: mol_type = unassigned DNA
tcaccaaccgccaagagagcttgatatgactgtatatagtatagtcataaagaacctgaacttgaccatatacttatgtcatgtggaaaatttctcatagcttcagatagattatatctggagtgaagaatcctgccacctatgtatctggcatagtgtgagtcctcataaatgcttactggtttgaagggcaacaaaatagtgaacagagtgaaaatccccactaagatcctgggtccagaaaaagatgggaaacctgtttagctcacccgtgagcccatagttaaaactctttagacaacaggttgtttccgtttacagagaacaataatattgggtggtgagcatctgtgtgggggttggggtgggataggggatacggggagagtggagaaaaaggggacacagggttaatgtgaagtccaggatccccctctacatttaaagttggtttaagttggctttaattaatagcaactcttaagataatcagaattttcttaaccttttagccttactgttgaaaagccctgtgatcttgtacaaatcatttgcttcttggatagtaatttcttttactaaaatgtgggcttttgactagatgaatgtaaatgttcttctagctctgatatcctttattctttatattttctaacagattctgtgtagtgggatgagcagagaacaaaaacaaaataatccagtgagaaaagcccgtaaataaaccttcagaccagagatctattctctagcttattttaagctcaacttaaaaagaagaactgttctctgattcttttcgccttcaatacacttaatgatttaactccaccctccttcaaaagaaacagcatttcctacttttatactgtctatatgattgatttgcacagctcatctggccagaagagctgagacatccgttcccctacaagaaactctccccggtaagtaacctctcagctgcttggcctgttagttagcttctgagatgagtaaaagactttacaggaaacccatagaagacatttggcaaacaccaagtgctcatacaattatcttaaaatataatctttaagataaggaaagggtcacagtttggaatgagtttcagacggttataacatcaaagatacaaaacatgattgtgagtgaaagactttaaagggagcaatagtattttaataactaacaatccttacctctcaaaagaaagatttgcagagagatgagtcttagctgaaatcttgaaatcttatcttctgctaaggagaactaaaccctctccagtgagatgccttctgaatatgtgcccacaagaagttgtgtctaagtctggttctcttttttctttttcctccagacaagagggaagcctaaaaatggtcaaaattaatattaaattacaaacgccaaataaaattttcctctaatatatcagtttcatggcacagttagtatataattctttatggttcaaaattaaaaatgagcttttctaggggcttctctcagctgcctagtctaaggtgcagggagtttgagactcacagggtttaataagagaaaattctcagctagagcagctgaacttaaatagactaggcaagacagctggttataagactaaactacccagaatgcatgacattcatctgtggtggcagacgaaacattttttattatattatttcttgggtatgtatgacaactcttaattgtggcaactcagaaactacaaacacaaacttcacagaaaatgtgaggattttacaattggctgttgtcatctatgaccttccctgggacttgggcacccggccatttcactctgactacatcatgtcaccaaacatctgatggtcttgccttttaattctcttttcgaggactgagagggagggtagcatggtagttaagagtgcaggcttcccgcattcaaaatcggttgcttactagctgtgtggctttgagcaagttactcaccctctctgtgcttcaaggtccttgtctgcaaaatgtgaaaaatatttcctgcctcataaggttgccctaaggattaaatgaatgaatgggtatgatgcttagaacagtgattggcatccagtatgtgccctcgaggcctcttaattattactggcttgctcatagtgcatgttctttgtgggctaactctagcgtcaataaaaatgttaagactgagttgcagccgggcatggtggctcatgcctgtaatcccagcattctaggaggctgaggcaggaggatcgcttgagcccaggagttcgagaccagcctgggcaacatagtgtgatcttgtatctataaaaataaacaaaattagcttggtgtggtggcgcctgtagtccccagccacttggaggggtgaggtgagaggattgcttgagcccgggatggtccaggctgcagtgagccatgatcgtgccactgcactccagcctgggcgacagagtgagaccctgtctcacaacaacaacaacaacaacaaaaaggctgagctgcaccatgcttgacccagtttcttaaaattgttgtcaaagcttcattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacagcctgggctgggggtggggtgggagaggtcttttttaaaaggaagttactgttatagagggtctaagattcatccatttatttggcatctgtttaaagtagattagatcttttaagcccatcaattatagaaagccaaatcaaaatatgttgatgaaaaatagcaacctttttatctccccttcacatgcatcaagttattgacaaactctcccttcactccgaaagttccttatgtatatttaaaagaaagcctcagagaattgctgattcttgagtttagtgatctgaacagaaataccaaaattatttcagaaatgtacaactttttacctagtacaaggcaacatataggttgtaaatgtgtttaaaacaggtctttgtcttgctatggggagaaaagacatgaatatgattagtaaagaaatgacacttttcatgtgtgatttcccctccaaggtatggttaataagtttcactgacttagaaccaggcgagagacttgtggcctgggagagctggggaagcttcttaaatgagaaggaatttgagttggatcatctattgctggcaaagacagaagcctcactgcaagcactgcatgggcaagcttggctgtagaaggagacagagctggttgggaagacatggggaggaaggacaaggctagatcatgaagaaccttgacggcattgctccgtctaagtcatgagctgagcagggagatcctggttggtgttgcagaaggtttactctgtggccaaaggagggtcaggaaggatgagcatttagggcaaggagaccaccaacagccctcaggtcagggtgaggatggcctctgctaagctcaaggcgtgaggatgggaaggagggaggtattcgtaaggatgggaaggagggaggtattcgtgcagcatatgaggatgcagagtcagcagaactggggtggatttgggttggaagtgagggtcagagaggagtcagagagaatccctagtcttcaagcagattggagaaacccttgaaaagacatcaagcacagaaggaggaggaggaggtttaggtcaagaagaagatggattggtgtaaaaggatgggtctggtttgcagagcttgaacacagtctcacccagactccaggctgtctttcactgaatgcttctgacttcatagatttccttcccatcccagctgaaatactgaggggtctccaggaggagactagatttatgaatacacgaggtatgaggtctaggaacatacttcagctcacacatgagatctaggtgaggattgattacctagtagtcatttcatgggttgttgggaggattctatgaggcaaccacaggcagcatttagcacatactacacattcaataagcatcaaactcttagttactcattcagggatagcactgagcaaagcattgagcaaaggggtcccatagaggtgagggaagcctgaaaaactaagatgctgcctgcccagtgcacacaagtgtaggtatcattttctgcatttaaccgtcaataggcaaaggggggaagggacatattcatttggaaataagctgccttgagccttaaaacccacaaaagtacaatttaccagcctccgtatttcagactgaatgggggtggggggggcgccttaggtacttattccagatgccttctccagacaaaccagaagcaacagaaaaaatcgtctctccctccctttgaaatgaatataccccttagtgtttgggtatattcatttcaaagggagagagagaggtttttttctgttctgtctcatatgattgtgcacatacttgagactgttttgaatttgggggatggctaaaaccatcatagtacaggtaaggtgagggaatagtaagtggtgagaactactcagggaatgaaggtgtcagaataataagaggtgctactgactttctcagcctctgaatatgaacggtgagcattgtggctgtcagcaggaagcaacgaagggaaatgtctttccttttgctcttaagttgtggagagtgcaacagtagcataggaccctaccctctgggccaagtcaaagacattctgacatcttagtatttgcatattcttatgtatgtgaaagttacaaattgcttgaaagaaaatatgcatctaataaaaaacaccttctaaaataattcattatattcttgctctttcagtcaagtgtacatttagagaatagcacataaaactgccagagcattttataagcagctgttttcttccttagtgtgt

SEQ ID NO: 47 - DNA, 29 bp, synthetic construct
  misc_feature 1..29: note = Synthetic sequence, primer1 (CCR5)
  source 1..29: mol_type = other DNA
ccacttggaggggtgaggtgagaggattg

SEQ ID NO: 48 - DNA, 25 bp, synthetic construct
  misc_feature 1..25: note = Synthetic sequence, primer2 (CCR5)
  source 1..25: mol_type = other DNA
taaagatgattcctgggagagacgc

SEQ ID NO: 49 - DNA, 25545 bp, Homo sapiens
  source 1..25545: mol_type = unassigned DNA
gcagtctttctgccacagcctcccaaagtgctgagattataggtgtgagccattgtacctggcttggagtaatgtttttgagcaaagcaaatgtacaggacatgcacagagaggcacagggagcacagagtccctggatgaatgctctggctggtggcagtattgtcacagggcaggctacccaaaggtgagcagaacacatgaggctagacaggtacaacttgctcatgaagagcctcgaatgccaagcttaggagtttgaactttattggaacactgacactaatagttaatgtttactgagtgcttactatgtgctaagagctatgcatgaatcacctcattctaattctcttaactctccacagcaggtcttattatttccagattgctgatgaaaaactgagtcacagagaggtaaaaaaaaaaaaacaaaaacaccctgggtccagagttgtgtgaaaaaattttaacaggttagagttgtgttgtagaaatctggtagccagccacgtgtaagacgaattgtagaaaggagagatcacaaatggctatcataagagaatcactgggaaaaggtaataagaacccaaattagaggcaaggagaacagaaagaaatgaaagaatgcaaagactgactggatgtgcacagcagtgaccgagagaaaggaggtatcacaggtgcctcttagctgctaagcctgggtgtcctgcagaaaggggcagctattatcagaagagagaagccagagagaggcacagatttggtggggatgatgttgagctccgtttgggggtatggtgagtgtctagggggcctgtaggaacccctccagaaaaattctcacaagcatttgaaaatcagtgacttgatctggagaaaaatatagggctggcattacaaacctgccggggtctgcattacagtattggttgaaagtgtggaaattgataatgatagatggttggtagatacatagatagatagatagatagatagatagatagatagatagatagatagatagatagaataaatagcatgatacatggaatgagggaagggctaaggataaagatgccttcatggatgcccacagttaggagaagaaagtagagcccatgaaggaacaactcccagagtttgcagatccaggcaagtatcttgttcagaaaataaagggaagggagtttccaaaagaaaagggggttgaaggtactcagatgctgaggagcctataaacaattgtgactaaaaaagactcaaatttggaaataaggtgtcctaatgaacatttcagaggatggagagagattcttaaagtcagcctttggaagaataactatatagaacacttccagaggccaaactagtgacaatggatcaaaagaattaaaatattgcatatctggaaattacacagctagaaatatgtagaaaagaaacacaaacatgcggtaagatacagctatggagctgtttgtcgaaattttgttaaaacagtgaaaatctgtaaactgtattaatgtccaacaataaaaaatatttaatgagaggcaaagatgctttgattggttataaagataaagaagttacaatactatgtacagtatgatcctttttttaaagggaggcacatatagaaagaactggaaggatatactgtaagaatttcacgatgtttgtttctgggtagtaggatttgacaattataagatctctgttttcccttcactccccccttttggattatctgtattttttataactctgtccaatagacatataatgtgagacacataattttaaatgttctagtggccacatttaaaaagtagaaagaaatagataaaataatcttcttaagatatttcattttactcaatatatccattactatgtgtaatcgatataaaaatattaatattttaaatgttttagcactaagcctgtttaacactaaatgcagtggatattcacatttacagaacatctctgtttgggccatccacttctcaagcaccacattaccgcatttggctcctagcacggctctatgaaataatagcagttaatatttatcaaaagcctactatgttccaggcactgctccaagttcatcaggtggagattataactcattaggtctcaataactccatgaggttgaacttattgtgctctccacaagcacgtgttgcttgcccttgtcctcataccataatgaaatgtttggagcttttgcaaaagccattttcctaatatgatgggcatccaagtctggttgcaaaggcttggatagagggcaagtgagagaattggagagagactaaaaatcattcaagaaattttgtggtgaaaaagacaagtcaaaagactgagagaaacatgaggacagaaaactctttgtaaggcaaggagacataaagatgaagtcaggctgaataggaaagaaggaggtgaaaggtgaaagagagatggctgggggtgtggctggtccaaattctcacaagatctctgttggagaaacattgcctggcttgtgctggtggcgagatgtacatagcagcttcccagctagagggagcagggagtcatggccttgtgagggagacagatggagacacacacaacccccatcgcaggtgaatgctatgaaagaatggggtggagggcttcccagggagatgaactgagttttaagaacgaagttagccagcaaagaagcaaaaggaagaggatatagacttgaggattacatcactggatgactgagccactgattggatgtggaggatgagagagtgatgctttgaggtgattcccaagttctccgcatggacaaaggcagagacaataacttgtactgagtgctctttggagtctgacattgatccagtgcaccacatatgttatctcatttactcctcacagaggtcccgtataacgcttgcaatttcacagatacgcaagctgaggcaaaataattggcccagggctcaccactaataaataggagagtctggatttggggccaggttattccagaaatagagagagggagggaaaacgttaatgtggggccagagccagaacaggtaggctaggctgtatcagcctgggaggcatcgggctattctccaaaaagtgaggctccgggttgcaggagatgggacctccaggaaactggtgggaggctggacaggggcaaagtgaaaggtaagcagcctgaagatgggtgggatgtagtggactccagcgtgaagagcctccagtctctggcagagctcagcagcttgggtgggaggaaacgaagggcagggagcagaaggtggtggggggaacgggttttggttaagatgggcagctgtccagtggtgaagaggggaagtatttgtgtatacgatcagttgtgggggatggtggtagagggagcagtatgagagtctgggaggaacagggcaggggagatggcacaggagaagattggggtgggggtgggggcactgatactgaatccagtacacagaggcaggcttagagaccccctgagggtgacaaattcttctcttaacttgctgcagaggaaacgacaagagaataggtcctaagaggagtgaaaatagaggaaactggccaagggataaagataagcagagctatggaagaaaaggaagaaaattagggaattactggagaggaagctgacagaaaggggcgctgggagaggaaaaggtgagggaggacgaaaacggaactcctatcacccagcgcattccagcaccccctccccctcccccgggaagtctgcgactgcatcctcacagggatggaatgggagtggaagctaggccaggctgggaccccggggctgtcccagcccaaccaagacggtgaccagactcaagttactgatcctccgctgtttcttcatctgtaaagtggagttaatgacagacagctggatgtcaacgttttttgttgtttgttttagagatgggatctccctgtatcgcccaggctggagtgcagcggtatgatcacggctcactgcagcctcgacctcctgggctcgagagatcttcctgtctcagcctcccgagtagaaatgtcagcatttgaatgcctcacaagggaaggtggtgtaataaaaggtctattgatttagatatgaacaagtatacccagagccacacgaatgaaaggaggtggccagtgtagatgcaagtctaggacggtcggtagattccaagcttctctacctgcccagttaaacaagttcgtggcccctggccagcctctcggtgggggtctgcgacagaacgggtgggattcattaccaggctaggagcgcaaggccctcgggccgggttcagcttgctgggctcctgtcttgtctcagcccccagcacttgctctgtcaccgcagagcacaggcccaggcaacgtttacccaactgaacccgcatccctgtgggaaccttgttccaacaaaatcccgtcgttgtctccttccatatggaacgaaaatctccttctgtacctctgcccagctccgcacccccgcccccttagagatacggcctggtcgggtcctggtccccccctcggcggcgcttccacttttcccagactgagggtggggaaaaggaggagggggaagaaatcagaggaggaaaagtcgggaggtaggggagccgggagacaggagagggggaaaataaagagcctgagacacaaacgagaggaaaagaccatcacagaaagctggaaatctccggagaggccagcgagaacccgcgctccccacggattccatcattccttccgaaggcgcctctgcggtgtctcagccgtgccaggccccggggttcccaggacgcggaggagtgctgggtgcggccgcctcgcctccccacccctggccgcccctccccacctcgcccaagggggccggaacggcgtcggcgcgcgggggcttttcggagcagtcgagtggaaaatagactttaacccgctttgtggcggccggggcgccctgagcgctctccaaaccacggctcccggcgctcaggcgggccgctgccaagacccggcctggagtccccgcagagttgcgcggcgcacggaccccgtggccttggggcgtcaggaggcccaacccagatctgcgcgcccaggcagcgctcaggccgctagaatggaccccggcagcggcgaggaagcggaactctctgcggctccctctccgcagtgcgccggcaaggtccaggtcccagcctccccaccgccgcccgcgccctcctaggcctcggagcggcgcctttctgcggcctcgaaggtggggtgggaaagtttggggagtcccggctctcacagcctgtcgtgagaactgcccccggggaattcgtccgccgtacggaaaaactggccggagcagagtcgtccgcggttccgcggtcgcgggtggaaggtgaaggtcgagggaggtcaggctgcttctgcgtgtcctgacggctggcgtgttctcttgagatgggctcgggctacttggccagcttcaatttaagccacagtgtctccgaggccctgacctggtccggcccgccgacacttgagcccccagagcctcagagaaggcgagggggtggatctcccagtgccgaggcccgccgtcctggtccaagccggtcgcggcaccgtgtctgggcactggagctgcttccagccccgcgaacagctggagggtggcagtgggaccgctccggcggcttctcccgcgcagtgccccgcctggccccttgtgaagggagtgagcgtcccctttccagagctgtcccccgtgacatccagaaaacgcgaaacctcaggaacaaggtcgcagcttcagaccgcggcccaggaggccgatggtgggtgagtgggagagtcccggagagcaggggggcagagagctggttttcgggaaaaccaatgtgttggaccccaaacatccaccctccgctcggatccaagttctctgagaactgaaacgacatcccgggacgaatgggagagttaggctgagctacacaccggggaggggagggttggagtttagccccaagcccttcggacgccttcttcggctcccgcgtgggttgagacggcggcacggccaccagactcagctaaagggcggagtcgcgaggagaagccagtggcgaggggaggaggaggcctggatctccccgcgaaggctccagtccggcttttgcctccgactgcgggctccctccccacccgccgtccctcgccccgccccgccccgccccccaccttggggcaggtgagcggcggccaatgggcgagcgcggggcaggtgcccgctaactcgcgcctcgcagcgctgggcggccggggctgggcagggcagtgcggggacaccgggggctggggtcggtcccagcgggactccgaaaggagggagacgagctcaaccctcgggccttactggcagctcgcagcctagcacggagcccgcgcctgtgcgggcgcctggagctgcccgctccgccgcagcagccgccgcgcctggccgtacgctgtggccggaccccgcggtcgctcgctcacacacccctcgccgctccgcgcctggctcgcccgcgggggccgagcgcgagcgggcgggcgggggaggtgaggggtgcgggcgggtgtgcatgtgcctggctgggtgcacaccccgcaaggcggcggcgccaggacgcggagcgctccccagagcccggctgcctcgcacagctcccgcggctgcgaccatgttccagcccgcggccaagcgcggctttaccatagagtccttggtggccaaggacggcggcaccggcgggggcactggcggcgggggcgcgggctcccatctcctggcggcggccgcctccgaggaaccgctccggcccacggcgctcaactaccctcaccccagcgcggccgaggcggccttcgtgagtggcttccctgccgcggccgccgcgggcgcgggccgctcgctctacggtgggcccgagctcgtgttccccgaggccatgaaccaccccgcgctgaccgtgcatccggcgcaccagctgggcgcctccccgctgcagcccccgcactccttcttcggcgcccagcaccgggaccctctccatttctacccctgggtcctgcggaaccgcttcttcggccaccgcttccagggtgagtgtccacgctgtgcccgccgaggcggccggccggcgcccgtgctgcggcgatgcgggggaggctcgggggcgcgcggggctgtttagaagttactgccgggaaggctgcaggtccgcggaggtagattcccaggcagggaagagctgtgcggcatccacccgcgccttcgccgcgtaggtctccctcccaggaaagcaggtggagacctccaggcttttctagaaaatataccagttcggacgcaagcccaggcgcgtcctcggagcctgtgctggccctcgccacagcctgcccaattctctctcccagctgagccagtctcagaccagagtacaactcctcccgctctccctccgcccggcttaacctcgcaccacgcttctctcgcaagtccaccaccacctccgagacctcagccttcgctggcgcgtccgggcgggggaaagtccattcgcgtgccccagctctgggggaagcaagggcagcagggagggcgaatcggagagttaatgttcagtgtggagggcctggctgtcttgggatgtttctcggcaaccttggcccgacttctccaagtcacacgtgcctctcctacccaaggtggggaaggtttgcagtaagcaaactggcttccgccgttgctcgccgccttcgggagggagcccacccggctgctggaataccgaggacagttttcccgggcagggggcgggggcagagggcttttaaggtcgtagccagtccgaaccccggagtttgcatccagcaatcggcttgctaataaagatcctccactggccctacacacacacacacacacacacacacacacacacacacgtttcaattatttgtctttcccggagaaaagagagttgcatttgttggagttcgttttcttccttgaaatttgttggagtttgtttttttcttttctttttttttaaattttattttaaagagtggccttgatttgtacaggcatcactttagtttccagttttattttgttagtgtagaccagaccacagccttgtgagaagggtctatggctcagagctaggtaacccggcttttagagaaacaaatgaaagggacatggctggagcttcggctccaggagctaatgtgacggtctgtagtctaggtctacagtcaattagatgtttggcacagttgtttagataataaaatgaaaattatctcttgacactttgactttcacagaaaaccgctttcccaggtcccgatttgtcaggcaattttttcagtcccacctggccaatagatgctgacctggcagataccacaaaaccagagaatgtaattactagaataagaattgttgtgggtagccttgcctcctctttgaagatttcaaagacttgcccaaatccaaatccgaaaaaacaaaaatgctacaatgtcatctgccttgggcaagagtttctgccacttaaaaataaatgtttactgataacatgaggatatctttaaaattgagcaatctaccctggtcctccgtgggctcgatccgaagcctgggtctcgaaacctggcgcccaggggccgagttgtagttggggcggtgtgtgagcccgcgggccgccgcggccgaggggctggcgggttggaggcttgtggagggataggggctcggaggagagggcggggtcgttcctaagtcctgtggcctccagccgttcagcttgtccggagtcggcatcctgggccgcaccctcggcttcgaatccagcccctgacgccctccgcaccgcggttcctgcctccgggcgccgagggccgggggcgcctggagagaaatccagctccggctctgagcgtctccagtcaggcgaggcggataaatccttcgcaaaaccctcttggaaattgccgccgcttcctgagccatcagtcccagcgggtacgttatcgagtagcacaaacagttggatttttccctcaagaaccgagtctggacgcggagatggagccaagtgtggctgcattttcggacccggaaatccgttgggcactgaaggacttttcgaaccctgtagcgctgttgcttcgcggtccatcgtcgccgctgcagacggatgcgctccccggcggctctacgccctccagtcccggccaggcctctgggctgggagccgagccgtctcgggccctccggcgccgcgttttctagagaaccgggtctcagcgatgctcatttcagccccgtcttaatgcaacaaacgaaaccccacacgaacgaaaaggaacatgtctgcgctctctgcgcagcgcttgggcggcgcggtcccggcgcgcggggaagcggcgtctccgctaaccgaggcgctggaaggggaaaagcgaatgcggaatcgtccaggactccgaaggtcggggccgctcgcgagcaccgaaggggaggagccgacgaagaccaggagtgggccgcatttcggtactgtttccccgagatcaggaactttccgggtctaggagcaacgcctggagggggctgtagagacccagccccccgggacccgcaactacaatgggccggagcttctaaggtcgcctttgttctggcaggaggacggggaatgaggttatctccgccgcctgtcctgcctctccctctcctagccctagggccctccgcccagccgtccggccctgagcccctggccggcggcggcctctccagcgaagactgcggctcgaagactgcagctcggaccccgggtgcttcggatccctagctcccacctccagctccctcattcctgggaatctcttgtgctagttcccagccattgccttgaaggggccctaaaagagtggctgtagaaaaatcggaggggtagggaagcagggaggagaggggtattcatttccctagctccagggacggctataccagtccctttccactttgctaactgtcctagtccgaaactgacagcccgttctcacagcccagaattactgcgtccaaacaggccgcaccctagacccaagtttgttctgcccttgtggtccaggcaagggaaactgaacccctggtaggggtggttcaggcctccttcccacaggtcggggggcggggcggtacaggtacctgtgcacctaaggcatcacccttgtctttgcagaaacatgtagcaattgatctgtttctcaggatgtttggtgttgtactaaacatcctctttctaacagggaaacgtccttattcttttggaatcaaataacctgtcatcacttagcatcttgactcatcctgcagtctcctgcttctctgtgatagggttagaaggacccctgtatttttgcacatgcatgtgaatataccctttaggacacatgctgtctaccacaactggacatgacaatgacctggggccattttctcagtaaggtagacccaaagcaacctagcatccccctaaaataaccagacttgaggcaaaggggcatgtatgttggtacagaagcttgttgccttcatcctctcatctgggtttataaagacaaacacagagcactccaccacacaggtgactgacatataccacataattacaaaataatcactaagtcagagacactggggcagactgcagacctgcttcctcagccccacactgcccttcacacctctgcctcctattcatacacacttacggggctttccacactgcagcctcacttctgaccaacctgggccagcccagcatctgaggccaaaccctgccaatgctgggatgagctaggctttctctctccctctctggttcatttgtccagaggaaaccactgttgggacttcacccaggttcataacaatgttgttttttgaagcaagttattaacattaacaagaagcatttgctttccacccacctttccctggcctacctcactggccccaccccagagactttaatcttccttattccccacctggagcaggctccatatttttctgccctttactcatctctgccagaccacctcccctgaccatctgtctattccactatcccaagtcaaacttctcttcagtcggacctgagggccctagatctgcgccacttgaataatcaaatggggtgtccctcacccatctccctgtgatgtggtcccaccatttttgtggctgcacagatccaaccagttgaaattgataaggtgactggagattattgactgaccccttccaggcactagccccgcaatcctagcaactctgttccacagaaaactccagcaaaaacttggcttctttaggtaaccaaagcccagagacttggaggaagtaaggtcagggagtttccaccaacagagggacaagaacagttacctggagagttttagctacagcatctcaattatctgcttttgattcacttacatagatgtttccagagatgggagatgttaactgaattatccaggtgattgtcttagagcaaagcaacaggtcaaatcaagtccaaccagtagccatctctgaagaaattaattggatcagtcaatcccaacagctaattctgtcaaaataatccatctagggttccgtgtttttggtgcatcaggaggctgttatgtgcccttacatgagaatccatgggtgattttgtcagggcctgttatgagtctgttagcatgtgcaaccaagccagagattgtgtgagggcctagtggggtgttcattgagacaggcgctgtgggtagaggcttgttttggcattgatagtttctgaccccatctcccctaccccagcttcatccagtccagtgttatcccttcctcctgcatgtgggagctgagtccctatgctggccaacatttgctaagggacagtcacactctaaaaatcctatacagtagataggaaaaaagacagtagggaggctggaacatatatttacacacgcccatgcagaaaccaatatatctatatctatatatattagagagaaagatgacatctagatatttatacacatgtttctttagcaagggactattcagggatgaagcagggatgaagcttttcccaccagacagtacttggagtctccagtgtgtgtgttgggatagggggttgggggcttaccctagaggctgggtctctggaccgccaaggcctgggggagagagaggtggagaaaggggaagaaggagcctgactttccacttccaggtgctgcctggaccactgacctagccactggcctatttataccccctgcaagacagagctagagcgtgctggcaggagtttttaatgagttagtagcctgagcattcagccgcaagactagtgcaagcaggtgtgaagggattgctccttgtgtaccttctagttcttgaatctgtgtttggcaaaggtgtgcctagcaccccgctgctccccttatcacgttcctgacccccagccctgcctctaccctgggtcctcttggagggagatgctttgcgaccagttaactgaaagcaaatcgttggggctggcggccagggcagcgcccctgggaaagggcggagaaagagcgccatggacttttcttccccaccccttggcctcttccgctgccccaggcattgtgaatgtgggtccacgcctcgtccggcctgccccatctcttggcttaacagagggatctggagagctgttattccccgcgttcccccgcggagtggctctcgagtgcggggaggtgttgcggaggggagtggacttagggaaggggcggcaaaagggcaaagggagaaatggcgtgtgtgtgcgtgtcaaggaatggagagggcagggcgcttgggagcagggcgcgaggccaggctctgttgggccccggctcacggcgccccttctctctgtctgtacctgcgtgtgttgccgtcggcggcggggccgcagccagcgacgtgccccaggacgggctgcttctgcacggccccttcgcacgcaagcccaagcggatccgcacggccttctcgccctcgcagctgctgcggctggagcgcgccttcgagaagaaccactacgtggtgggcgccgagcggaagcagctggccggcagtctcagcctctccgagacgcaggtaatcacccccggtcgcggcctgccctgcgcccggagcccgggtggaggtgagggtgcgcgggtgcaggagaggccctgagcccgccccagcccagccctgctgggttccaaaaggcccccattccccgcggcgctgcggtcaagcccgtctttagagcctcttcctcgagactgcgtgcagcctgctgagcccgcaggacttttgtcaagcgctaaagacctagcaggaggcagagtaaatgcaaactgtatcccgagcccggctcccaaagctcctcacggggggaccaggttccctggaggaagcgggtcgcctcgggagcgggcagcgcaggcagcaccgaggccactggagctggctccagccctggcattcctgcagcccttttcccgccactgtgtcggggcgctcatagtcctgcggggagccggtccgcactggctttgctgctgttcctgggcaaaactggcggggccttggctgcccaccagccaggagcgtctggggagaaagcccaggtgtcctcagactaccaacagaggggcttaaccagggaggggccagcccctgcttggggcccgagggttgctctgatccggcccaggccggctgatagggctgtggaagccacggtgtgcgcgcgcagagcatctgagtggcctgggcctggtgggaaatagaccccgggtactcaggtgcttctctgaatcactggaaaggctgtcgaatgggagaaggaataaactccaacggcgcctgggcttgaactgagtgaaattaacaattaccgtgtagtgtttttgtaactgatcgttaatttaagggaaaaaattaaagaattagatgaaagttatagggaggtggatttgggttcattgtaagtagactttgccataaataaatgctgcctgggatcactgcataagctcttggtccacccaggtccgacgtgttggagtggggctcagcgaccctcagcctagctgctgccctggaggtggatttcagtctctgcgtgccggccggctcccagagttgcgagaggccggctccgcggtctcccagctacctcccggctgacttttcaccttccgctcccctttcctcctagtctcgaccctactacaccaccgtcccctcccaagtcccgggcagtgagaagatgcccggcatggggggcagccggagcctccctttagcagccagagtaggaagggggcttagtgagggagcccagacccaaacttcatccgcagctttcttcggcggaccttaccctctcctccttcagtggcattttggcatctattgtcgtcatatctgtctgctgccccacttaatctacaaatcgctcacgggtcggaggcaggacccgtgcgttttcagatgtactagctgggctgttctaactgcagggaaaaagcttacaaaacaagagttaattttaaaaacgtttcaaagaaagatgtgtttttaaaaataagttaataaaataacactcccttttccctcctggcagtgttttaaaattattgtttgaaacaaggtgtcagtttaagaatggtgtttataattaacttcatttaaacagtaatatttattaaattttaattgcagaactgtaagaaaacaaaaatggtttttaatcctaccacccacagattaacacttgttgaaataatgtcattgtttttaaactttcaattttttagctcagtgagagcatttttaattaactctctttcaaactgaacctagctgcctgtcaatatttgctcctaacaatgccagttagtaaacggactgattgtttctttcatttttattatgagacatttcaaacatatatttaaaacaaaaccagatagaataacataaacgtatcctgcttcattaactatcaaaactcagggctaatcttcttcccgtctgtaaatgagctgctttttgcatatggtacaacaaaaagaatgaggggaggtttgagcctggggaacctgccgtggcagcctgtccttccaggtgaagaccctgagatggagagatggtttggacagagcttcccaggtgggcaaacacgattttaaatacctgcctccctgctaacttactgtgtaaccctgggcaagtcacttaacctctctgagctctggtttcctcattgagaaaatagttgtttaatgattaaaggtacctcatatgagctcaaataatattaatccccatccctagtcccttccctttaaggtagtatgtcagattagtagcataagaagatccaaacctgtgtgtcctcttagtccagcgttcttcctcccttatgcagtttccgtcatcaacattgcctttctgttgccctcataacttatcatacggagccaggctatataattagctacttctctatccgcctcttggtacttaccaggcaagcttacctgtcatttccagctatcagctatttgtcaagcatcagtcacccccaacagcccccctgccatgcatttctactgttaatatctgtatctgtctccagagctctctaatctgtcccaccccgtagctgttttacatctagccatatacttctgtctgctctcattaaactgcctccccaaaactggctaatttatatttccgattttctattagttatatgttcttcctaacaataagaactatttacccatttgttcattatctttgtcatctattttaccttctgtgtttccaccattcatctcaaacttcatctccatagagctatttcgcaactgacaaacagtatattcatatgtctggtaatggttatctattattggctctggctttgtcttctctctgggtctgggcctcagtttctgtatctgtgagattgaatgacacaaattccagggttttttctagtgctgagtttctgtgactcctctacattctacttctctgtgtttctgtatactacctcctccacattctcagagctcaccacacaacccctgcctatcatgatatgcatcaaactttgttgttattacttaattatctgccatgtccaaacatcaatctgtagaccagcagtatgcgtctctcagggagatcttaaaatacagattcctgggtttcacccaggagattctcttaggaagtccaggatagggtgcaggaaaaagtttaaaaacagttatttgggtgatcatgattaataacaggcctgagcatcttagctctgggaggcagaggccaagcctgtctgtttcttacaggacccagctcagtgcccgggatggagtacatgctcaataaacatgtattgaattaatgagcacatttctctttgcccatacaaatacacactaactttatcagtcattccccttgctctctgctgtcattgctccctccctgtccctctccttctatctttcccttgtactttcacagctgattgttgatttagattatgcatataccagtttgtggataaaacttctcggagggttactcagatcagtgtgtgaatgagctcttaatccagatctcagaagtctgtgcactccccaagctttagccgggtgctaggaggtgggcaacctgggtgactctgtgtgtttagtgggagtggggtattcgtgctgggatggccagtgcctcaatctaggagatgagggaagagccctgggcaagggctagttctcccttcaggttctaatgacttgttcctcactgcttgggtgccgccctggagtatgaccaggaaggtaccagtctaagcttcagtcctggtggctggttgggcagacctgggcctgggtcattgcagaggctcaagtttaatgagtatgtgtaatgggtgtgtgcaacatgtgtctgcccatgtggggcaccaacgggctttatgtgattgatgcccaaaggtcagatgatagcataggtacacattagatgccattaggcagtcatatgacatggagtgcagcttgcatgcttttgtgtgtgttcgtgtgtgtgtcgggggcaggggtaagttagttttagggggagtgagagaaagcacctggttgctccaggctgatcaactggtcagtgtttccagctactccttctgctctgaacagatcagcaggtgtttcttgaccttgcctgggttagagtttagctgagcggtgaaggcaaagggtacagaaacgtggcctgtggctttgaagatttcttactgagtgatgaaggctaagtgcaaagcttgcacatttgtgaaacatgcacaggaagaatgactagggtcccctttgaggtcacagctgtgggctgaggggtgtcaggatagaaacgcttgagaagatggctccaggaggcctcagacctggaagacttggggggatgcctaggatcctaggttggagggaaagaagggcagggtttgaggcaggcagatgaagatagagccaccatcttggagcccaagggcagggagatcttgggaggcaggagttacggtcacctgcctatggctttttcccctcagaggcatggaaaggaggatttggagggtcctttcctgctctggaatgttctggccttagagggatggataagagggggatatccaagtgacctgaattttagggaaaaatcaagagacatttgttcctagctcacggtgtgtccacatctcttctctaagtcttggcttttcttcaagaacttctgcatctcatgttccaggagtcctgtgtgggaggatgagggggagataaaggagattagagtggttctgtgaggagctgggaccaacatgtcctgaggtgaatcttatgatgtctccctgagagagacagaggaagggtccaggctgggctgaaagaggaggagacagggaggcttagggagatcatggttatggttggtggggagctggaggtgacctcagcttactgaggtgaaggttgaacttagcatggcattgattgggcttgaccttgagagtgggaacagcccacatgatcaaatgataggaaatgggtccccagggaagagagaaagctgaggggtgacttgactgatatctccaggctcctgtccattctgggatatttgaaatctttgaagacaggacacgtattcacctgaaagtgtgcaggggcctggagccacagacttttccatttgcaggagtggtgaaaaagaagggatccagacctcgttcattcagaatagcagattattccaaagtaaagtctgattagttctgaaatatcggctggagccatagtttgcttgtttgttagttcatttttccctttgttcttgcatgcatgtcttcatttattaatgcatacattgatcagtcctctagcaaactgatacattcattcatccatagcagtttcaccttcttcattctgcctgactcaagccaacccttcttctgcccagcagtaggtgtccctcctccaacttcccctaaaagtggccaatccaatttaccatgtggaatattaaaaactggccctcttgcaaaagtgtccacaaaactaagaaaaagatccagtttctccatcattgagcacttctcaaagcctttgctgattagaattctacccctcttctgttcattttctcctgttttccaggtctggcccaggtacctcttgcctagagcataggcttgtccagccagatgactatacatggaacatgtcctctgggcagggcactgggactaacataacagtttggctctccagtctcatagtctggtgaggaggcagacgtaaataaataaattagtgcacagtgggggtcactgttactgagactgggaagaggtactaaggaactacaggcaggtgatggggcaggaccattaaggaacatcaggacagggtcttgaaggctaaggagagtattccagggttggtaagtcaggagaatggagacttttggaaggatgtggcctctgagaaactgagatgttcacttcacagtgaaatgaggacttgggagaccacaagggacctcttgctacagcagagaggtcagagtgggcaaggggtcctccagctaacattccaactgtacctcggggcttagagagaggtgagtagtgtgtttgtgttggggatggggaggcatgtgcaggacagatccagcctccaagccataccatatgacccagctcctttccagggcctgttttctctagggaaggggctctgaaggatccagagttgcctggctgggttggagaggtcatgaaatgtcactctcatctcctaatacactcagagcccagttccttttctttttccaagtaaaaaataactaccattatcaataatctattaaggaaaattcagaaaagtagaaagaagaaagaaacatcacccacactttaggtatatttccttctagtcttttttccccgtgtgtagattttgtttttctatggttgtgatcacactgtgccagccttgtgcatcctggcgtttccattaatgctatgaagtcataagcactaccctgttattgcagtctttgtaaacagcatctggacactcagcccaaggctgttctccggaagatggccaggctgcacagggagagggtttatgtccctgcctctagagagatgcctacttgaccccagtatttttcatggagaaaatattcagaatcacctttcacttgggtgccctaggaagctgcctctggcctatcctgtgcctgaagtcgccatccaaagctttccttctttgagccagtgttgctagtcaagggcagcatgctgggcccgtcccactacaggccaatgtgaccgtcagtctccttcctgaaggacacttggaaatgcatgtggaaagaggaaggtacagaaaagggcccccggtccctggtactgcccgcatcacctgacagtcaccttcggccagcccacttgggcttctcaggaatgacaccccggccctgcatctggccctgagtcacgcacaggaggcagggtgagctcacccgcccactgactggcacagtcatagcaggctccagggtggggggcaggggccaggctgctgccaaatggttgtgctgagaaccacccagggtccaggtggccctgcgcccaaagataggtgggcttggagtccagcagcctgtgcccagagcctctgccatcctccacggccggcctaggtgagatgtgcaggctatgggcttggaaatgaggggagaatccccttgccctcactccatccatcgaggccgggcaccaacccttcctgggccagctttccagcccctggctggctgctctgcaggcactgaatgccagctgcccccatccccatgccagtgctctaaaatcagtgctctcaaacaagggcagatggcgcagtggaagttctggcaagaggggactgtgaggggaagtcctggggtaggtgggacagagaggactgcctgggaagggtgtaggggcagcacctcctgggcatgaaaccatctgcagggcacaggggccaggcctgcctctgcatgcactgcttggccttgtggctaaggcctgtgctttacccagttctctgggagcaggagcagtctttctgaggcctgccctcagccctgcccagggttcaaggatctctcctcagcatcattgctgctgccaaaaccaagaggctacccaccattcctcacccgcaactctgccacgcagcaccgtctgcacagctgcagttggccacatccacttgcttttaaatgtgctgtcatttcctggaaaccatccaggccttgtagcctgccctctgcacctcctccccaaggggggcctctggagcaagaatccaagaggtgccctggcagctgcaagtgtccccagaacatttcctctatgcatgaaggactgaagcccagagggggaagggacttagctgagaatcagtacccaggatcctcctgccaacgctgaagatggtttgggtctggcctgactctgcaaagccaagtaaagaaccacggagtcagggagagttgacagatgaaggctttctccacagcccacaagcactaagtccagtccaggagccaggatgagcctcccagattatgcatgagaggacattaagggctgtgtcctggacactgaacagatgctggaggaggagggaaaagaggcttcctggaggagatggcttagaaccatagacctgccctctgcctcatgcctcctccatcatggaggatggttgtccagtttctgtttgaaccccaccccataccttgccagggctctcactgccagacacagaatagggggctccctgggttcaaagtagagctcacttctgtcccctgggcttctcctgactgttccttgtgtgacctgttcccacatctggatgggctgcaggagccagtgctgtggggacagaaggtctggagctgcccgtgaagggcagaatgctgccctcagacccgcttcctccctgtccttgtctgtccaaggagaatgaggtctcactggtggatttcggactaccctgaggagctggcacctgagggacaaggccccccacctgcccagctccagcctctgatgaggggtgggagagagctacatgaggttgctaagaaagcctcccctgaaggagaccacacagtgtgtgaggttggagtctctagcagcgggttctgtgcccccagggatagtctggctgtccaggcactgctcttgatataaacaccacctcctagttatgaaaccatgcccattctgcctctctgtatggaaaagagcatggggctggcccgtggggtggtgtccactttaggccctgtgggagatcatgggaacccacgcagtgggtcataggctctctcatttactactcacatccactctgtgaagaagcgattatgatctctcctctagaaactcgtagagtcccatgtctgccggcttccagagcctgcactcctccaccttggcttggctttgctggggctagaggagctaggatgcacagcagctctgtgaccctttgtttgagaggaacaggaaaaccacccttctctctggcccactgtgtcctcttcctgccctgccatccccttctgtgaatgttagacccatgggagcagctggtcagaggggaccccggcctggggcccctaaccctatgtagcctcagtcttcccatcaggctctcagctcagcctgagtgttgaggccccagtggctgctctgggggcctcctgagtttctcatctgtgcccctccctccctggcccaggtgaaggtgtggttccagaaccggaggacaaagtacaaacggcagaagctggaggaggaagggcctgagtccgagcagaagaagaagggctcccatcacatcaaccggtggcgcattgccacgaagcaggccaatggggaggacatcgatgtcacctccaatgactagggtgggcaaccacaaacccacgagggcagagtgctgcttgctgctggccaggcccctgcgtgggcccaagctggactctggccactccctggccaggctttggggaggcctggagtcatggccccacagggcttgaagcccggggccgccattgacagagggacaagcaatgggctggctgaggcctgggaccacttggccttctcctcggagagcctgcctgcctgggcgggcccgcccgccaccgcagcctcccagctgctctccgtgtctccaatctcccttttgttttgatgcatttctgttttaatttattttccaggcaccactgtagtttagtgatccccagtgtcccccttccctatgggaataataaaagtctctctcttaatgacacgggcatccagctccagccccagagcctggggtggtagattccggctctgagggccagtgggggctggtagagcaaacgcgttcagggcctgggagcctggggtggggtactggtggagggggtcaagggtaattcattaactcctctcttttgttgggggaccctggtctctacctccagctccacagcaggagaaacaggctagacatagggaagggccatcctgtatcttgagggaggacaggcccaggtctttcttaacgtattgagaggtgggaatcaggcccaggtagttcaatgggagagggagagtgcttccctctgcctagagactctggtggcttctccagttgaggagaaaccagaggaaaggggaggattggggtctgggggagggaacaccattcacaaaggctgacggttccagtccgaagtcgtgggcccaccaggatgctcacctgtccttggagaaccgctgggcaggttgagactgcagagacagggcttaaggctgagcctgcaaccagtccccagtgactcagggcctcctcagcccaagaaagagcaacgtgccagggcccgctgagctcttgtgttcacctgcccttctgtttgtcccacttgtcaggatgaaggtttcctgacaagcaaatctgcattcctaagtctttcccttacgacatccagacccctctctttcttcttcacctcccatgtgctcatgaaacctctgctctttggcctccatgccaccattctgccggtgctaatgacagtcaccaaccacagtcactggccacccccttgtggccaaactcaaccacttcctgttgggttcacctgctccctgatctgttggctatgccctcctggattctcccaccccgctctgccttcatttcggtagctctgactcctccttcagcctgagttcattcagtgtgatccatgccttgtcacctctggccacccccaggtcagtccttgctctccctgactcctcattatgtcctttgccttgtggggatcacatccacttccaggacttctctttggctccctggcctgtgtctttagctgacctctgctgagctgcagacccgtcaagccagctgcctgctccaccccctgcccctcagcacctcaaagcccatacatccacagccggacgcacctacccatctgtgcttctccaggatcacccacctcgacaaacagcattagacatgtacacagtttcccaagctacaggaaatctgggaatcttcctcaagcttcctctcctcttccccagccccatccattcccatctagtgggttgagtctagactggcttcccaaccacacctccctaatgcagcaccccctacccccctgcccagctccctctgccaccggcctggcctgggccctgctcctctagggaggtttctgtgaatgtcaaggatgaggtccacttcaaaggaggccctccatgttgcagctaggtttctcttccttgcacccaaacagggcagactcacccccttctgagcccctttgccccatcttcttgccttgctcccggtaccctttgcttcagcatccccatccctgtccctgagtgagccacaggttttccctgtgctgtgccttcgctcatgctgttcttcccacctggaatgtcgggtgcttgatcaatgtggaactcactggaaagatgtcagagacccagccttgctgtctgggccacatgcagggatccaagcacacaaggtccttctgctgggagcacagacccaggtcccactcgcactctcagcgtctctctcccacctctgcccacctcacttgtgtccagtcagtgctagaaaccaaagggctttgtcccatcccaacaccccctctccctccatcagtcaggaatgcattctgcacatcttgaaagtcctaacatggataagtccagattaacccacatggcgaccctcactgccaagcaggtgggatcacttctgggagcacacatgcccaggtgtggaaggaaggtgggaggaagagtcatccttttggccccagtgggggacagagaaaggggtgagctgttcctctcaagatcctgcctcacttggtagggggagggggtccaggaagatcacagcagagcaccccctgtcatccgaataaagggctggaagggacccaaggaaacctgccagtctcccgaggccaggcctgcggggggcggggcggggagtccctgccgcactcccatccaccccccatgttgtgcctctccctgcagacggtaaatattggtgttgtgacttcattaataaaggcttctgtgagcctgaaaaa

SEQ ID NO: 50 - DNA, 30 bp, synthetic construct
  misc_feature 1..30: note = Synthetic sequence, primer1 (EMX1)
  source 1..30: mol_type = other DNA
gggcttctcctgactgttccttgtgtgacc

SEQ ID NO: 51 - DNA, 30 bp, synthetic construct
  misc_feature 1..30: note = Synthetic sequence, primer2 (EMX1)
  source 1..30: mol_type = other DNA
caggatggcccttccctatgtctagcctgt

SEQ ID NO: 52 - protein, 7 aa, synthetic construct
  REGION 1..7: note = Synthetic sequence, NLS (protein)
PKKKRKV

SEQ ID NO: 53 - DNA, 21 bp, synthetic construct
  misc_feature 1..21: note = Synthetic sequence, NLS (nucleotide)
  source 1..21: mol_type = other DNA
cccaagaagaagcggaaggtg

SEQ ID NO: 54 - protein, 19 aa, synthetic construct
  REGION 1..19: note = Synthetic sequence, BPNLS (protein)
KRTADGSEFESPKKKRKVE

SEQ ID NO: 55 - DNA, 57 bp, synthetic construct
  misc_feature 1..57: note = Synthetic sequence, BPNLS (nucleotide)
  source 1..57: mol_type = other DNA
aagcggactgctgatggcagtgaatttgagtccccaaagaagaagagaaaggtggaa

SEQ ID NO: 56 - DNA, 44 bp, synthetic construct
  misc_feature 1..44: note = Synthetic sequence, BPNLS insert upper
  source 1..44: mol_type = other DNA
agatcttaatacgactcactatagggagagccgccaccatggcc

SEQ ID NO: 57 - DNA, 13 bp, synthetic construct
  misc_feature 1..13: note = Synthetic sequence, BPNLS insert lower
  source 1..13: mol_type = other DNA
taatatcctcgag

SEQ ID NO: 58 - protein, 22 aa, synthetic construct
  REGION 1..22: note = Synthetic sequence, 2A (protein)
GSGATNFSLLKQAGDVEENPGP

SEQ ID NO: 59 - DNA, 66 bp, synthetic construct
  misc_feature 1..66: note = Synthetic sequence, 2A (nucleotide Cse1-Cse2)
  source 1..66: mol_type = other DNA
ggaagcggagcaaccaacttcagcctgctgaagcaggccggcgatgtggaggagaatccaggcccc

SEQ ID NO: 60 - DNA, 66 bp, synthetic construct
  misc_feature 1..66: note = Synthetic sequence, 2A(nucleotide Cse2-Cas7)
  source 1..66: mol_type = other DNA
ggctccggcgccaccaatttttctctgctgaagcaggcaggcgatgtggaggagaacccaggacct

SEQ ID NO: 61 - DNA, 66 bp, synthetic construct
  misc_feature 1..66: note = Synthetic sequence, 2A(nucleotide Cas7-Cas5)
  source 1..66: mol_type = other DNA
ggatctggagccaccaatttcagcctgctgaagcaagcaggcgacgtggaagaaaacccaggacca

SEQ ID NO: 62 - DNA, 66 bp, synthetic construct
  misc_feature 1..66: note = Synthetic sequence, 2A(nucleotide Cas5-Cas6)
  source 1..66: mol_type = other DNA
ggatctggggctactaatttttctctgctgaagcaagccggcgacgtggaagagaatccaggaccg

SEQ ID NO: 63 - DNA, 1388 bp, Homo sapiens
  source 1..1388: mol_type = unassigned DNA
tgaatgggtatgatgcttagaacagtgattggcatccagtatgtgccctcgaggcctcttaattattactggcttgctcatagtgcatgttctttgtgggctaactctagcgtcaataaaaatgttaagactgagttgcagccgggcatggtggctcatgcctgtaatcccagcattctaggaggctgaggcaggaggatcgcttgagcccaggagttcgagaccagcctgggcaacatagtgtgatcttgtatctataaaaataaacaaaattagcttggtgtggtggcgcctgtagtccccagccacttggaggggtgaggtgagaggattgcttgagcccgggatggtccaggctgcagtgagccatgatcgtgccactgcactccagcctgggcgacagagtgagaccctgtctcacaacaacaacaacaacaacaaaaaggctgagctgcaccatgcttgacccagtttcttaaaattgttgtcaaagcttcattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatag

SEQ ID NO: 64 - DNA, 987 bp, synthetic construct
  misc_feature 1..987: note = Synthetic sequence, CCR5 Gene with CRISPR induced deletion.
  misc_feature 76..476: note = Deleted nucleotides
  source 1..987: mol_type = other DNA
ccacttggaggggtgaggtgagaggattgcttgagcccgggatggtccaggctgcagtgagccatgatcgtgccactgcactccagcctgggcgacagagtgagaccctgtctcacaacaacaacaacaacaacaaaaaggctgagctgcaccatgcttgacccagtttcttaaaattgttgtcaaagcttcattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatcttta

SEQ ID NO: 65 - DNA, 987 bp, synthetic construct; identical to SEQ ID NO: 64
  misc_feature 1..987: note = Synthetic sequence, CCR5 Gene with CRISPR induced deletion.
  misc_feature 120..460: note = Deleted nucleotides
  source 1..987: mol_type = other DNA

SEQ ID NO: 66 - DNA, 988 bp, synthetic construct
  misc_feature 1..988: note = Synthetic sequence, CCR5 Gene with CRISPR induced deletion.
  misc_feature 115..382: note = Deleted nucleotides
  source 1..988: mol_type = other DNA
ccacttggaggggtgaggtgagaggattgcttgagcccgggatggtccaggctgcagtgagccatgatcgtgccactgcactccagcctgggcgacagagtgagaccctgtctcacaacaacaacaacaacaacaaaaaggctgagctgcaccatgcttgacccagtttcttaaaattgttgtcaaagcttcattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgctaaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatcttta

SEQ ID NO: 67 - DNA, 987 bp, synthetic construct; identical to SEQ ID NO: 64
  misc_feature 1..987: note = Synthetic sequence, CCR5 Gene with CRISPR induced deletion.
  misc_feature 61..404: note = Deleted nucleotides
  source 1..987: mol_type = other DNA

SEQ ID NO: 68 - DNA, 2130 bp, Homo sapiens
  source 1..2130: mol_type = unassigned DNA
agggaaaagaggcttcctggaggagatggcttagaaccatagacctgccctctgcctcatgcctcctccatcatggaggatggttgtccagtttctgtttgaaccccaccccataccttgccagggctctcactgccagacacagaatagggggctccctgggttcaaagtagagctcacttctgtcccctgggcttctcctgactgttccttgtgtgacctgttcccacatctggatgggctgcaggagccagtgctgtggggacagaaggtctggagctgcccgtgaagggcagaatgctgccctcagacccgcttcctccctgtccttgtctgtccaaggagaatgaggtctcactggtggatttcggactaccctgaggagctggcacctgagggacaaggccccccacctgcccagctccagcctctgatgaggggtgggagagagctacatgaggttgctaagaaagcctcccctgaaggagaccacacagtgtgtgaggttggagtctctagcagcgggttctgtgcccccagggatagtctggctgtccaggcactgctcttgatataaacaccacctcctagttatgaaaccatgcccattctgcctctctgtatggaaaagagcatggggctggcccgtggggtggtgtccactttaggccctgtgggagatcatgggaacccacgcagtgggtcataggctctctcatttactactcacatccactctgtgaagaagcgattatgatctctcctctagaaactcgtagagtcccatgtctgccggcttccagagcctgcactcctccaccttggcttggctttgctggggctagaggagctaggatgcacagcagctctgtgaccctttgtttgagaggaacaggaaaaccacccttctctctggcccactgtgtcctcttcctgccctgccatccccttctgtgaatgttagacccatgggagcagctggtcagaggggaccccggcctggggcccctaaccctatgtagcctcagtcttcccatcaggctctcagctcagcctgagtgttgaggccccagtggctgctctgggggcctcctgagtttctcatctgtgcccctccctccctggcccaggtgaaggtgtggttccagaaccggaggacaaagtacaaacggcagaagctggaggaggaagggcctgagtccgagcagaagaagaagggctcccatcacatcaaccggtggcgcattgccacgaagcaggccaatggggaggacatcgatgtcacctccaatgactagggtgggcaaccacaaacccacgagggcagagtgctgcttgctgctggccaggcccctgcgtgggcccaagctggactctggccactccctggccaggctttggggaggcctggagtcatggccccacagggcttgaagcccggggccgccattgacagagggacaagcaatgggctggctgaggcctgggaccacttggccttctcctcggagagcctgcctgcctgggcgggcccgcccgccaccgcagcctcccagctgctctccgtgtctccaatctcccttttgttttgatgcatttctgttttaatttattttccaggcaccactgtagtttagtgatccccagtgtcccccttccctatgggaataataaaagtctctctcttaatgacacgggcatccagctccagccccagagcctggggtggtagattccggctctgagggccagtgggggctggtagagcaaacgcgttcagggcctgggagcctggggtggggtactggtggagggggtcaagggtaattcattaactcctctcttttgttgggggaccctggtctctacctccagctccacagcaggagaaacaggctagacatagggaagggccatcctgtatcttgagggaggacaggcccaggtctttcttaacgtattgagaggtgggaatcaggcccaggtagttcaatgggagagggagagtgcttccctctgcctagagactctggtggcttctccagttgaggagaaaccagaggaaaggggaggattggggtctgggggagggaacaccattcacaaaggctgacggttccagtccgaagt

SEQ ID NO: 69 - DNA, 1730 bp, synthetic construct
  misc_feature 1..1730: note = Synthetic sequence, EMX1 Gene with CRISPR induced deletion.
  misc_feature 163..675: note = Deleted nucleotides
  misc_feature 1030..1392: note = Deleted nucleotides
  source 1..1730: mol_type = other DNA
gggcttctcctgactgttccttgtgtgacctgttcccacatctggatgggctgcaggagccagtgctgtggggacagaaggtctggagctgcccgtgaagggcagaatgctgccctcagacccgcttcctccctgtccttgtctgtccaaggagaatgaggtctcactggtggatttcggactaccctgaggagctggcacctgagggacaaggccccccacctgcccagctccagcctctgatgaggggtgggagagagctacatgaggttgctaagaaagcctcccctgaaggagaccacacagtgtgtgaggttggagtctctagcagcgggttctgtgcccccagggatagtctggctgtccaggcactgctcttgatataaacaccacctcctagttatgaaaccatgcccattctgcctctctgtatggaaaagagcatggggctggcccgtggggtggtgtccactttaggccctgtgggagatcatgggaacccacgcagtgggtcataggctctctcatttactactcacatccactctgtgaagaagcgattatgatctctcctctagaaactcgtagagtcccatgtctgccggcttccagagcctgcactcctccaccttggcttggctttgctggggctagaggagctaggatgcacagcagctctgtgaccctttgtttgagaggaacaggaaaaccacccttctctctggcccactgtgtcctcttcctgccctgccatccccttctgtgaatgttagacccatgggagcagctggtcagaggggaccccggcctggggcccctaaccctatgtagcctcagtcttcccatcaggctctcagctcagcctgagtgttgaggccccagtggctgctctgggggcctcctgagtttctcatctgtgcccctccctccctggcccaggtgaaggtgtggttccagaaccggaggacaaagtacaaacggcagaagctggaggaggaagggcctgagtccgagcagaagaagaagggctcccatcacatcaaccggtggcgcattgccacgaagcaggccaatggggaggacatcgatgtcacctccaatgactagggtgggcaaccacaaacccacgagggcagagtgctgcttgctgctggccaggcccctgcgtgggcccaagctggactctggccactccctggccaggctttggggaggcctggagtcatggccccacagggcttgaagcccggggccgccattgacagagggacaagcaatgggctggctgaggcctgggaccacttggccttctcctcggagagcctgcctgcctgggcgggcccgcccgccaccgcagcctcccagctgctctccgtgtctccaatctcccttttgttttgatgcatttctgttttaatttattttccaggcaccactgtagtttagtgatccccagtgtcccccttccctatgggaataataaaagtctctctcttaatgacacgggcatccagctccagccccagagcctggggtggtagattccggctctgagggccagtgggggctggtagagcaaacgcgttcagggcctgggagcctggggtggggtactggtggagggggtcaagggtaattcattaactcctctcttttgttgggggaccctggtctctacctccagctccacagcaggagaaacaggctagacatagggaagggccatcctg

SEQ ID NO: 70 - DNA, 1730 bp, synthetic construct; identical to SEQ ID NO: 69
  misc_feature 1..1730: note = Synthetic sequence, EMX1 Gene with CRISPR induced deletion.
  misc_feature 296..989: note = Deleted nucleotides
  source 1..1730: mol_type = other DNA

SEQ ID NO: 71 - DNA, 176 bp, synthetic construct
  misc_feature 1..176: note = Synthetic sequence, pre-crRNA(LRSR)
  source 1..176: mol_type = other DNA
tggatgtgttgtttgtgtgatactataaagttggtagattgtgactggcttaaaaaatcattaattaataataggttatgtttagagtgttccccgcgccagcggggataaaccgcaggccaatggggaggacatcgatgtcacctcgtgttccccgcgccagcggggataaaccg

SEQ ID NO: 72 - DNA, 90 bp, synthetic construct
  misc_feature 1..90: note = Synthetic sequence, pre-crRNA(RSR)
  source 1..90: mol_type = other DNA
gtgttccccgcgccagcggggataaaccgcaggccaatggggaggacatcgatgtcacctcgtgttccccgcgccagcggggataaaccg

SEQ ID NO: 73 - DNA, 61 bp, synthetic construct
  misc_feature 1..61: note = Synthetic sequence, mature crRNA
  source 1..61: mol_type = other DNA
ataaaccgcaggccaatggggaggacatcgatgtcacctcgtgttccccgcgccagcgggg

SEQ ID NO: 74 - DNA, 35 bp, Homo sapiens
  source 1..35: mol_type = unassigned DNA
aagcaggccaatggggaggacatcgatgtcacctc

SEQ ID NO: 75 - DNA, 34 bp, Homo sapiens
  misc_feature 1..8: note = Seed sequence
  misc_feature 9..32: note = Spacer sequence
  source 1..34: mol_type = unassigned DNA
tcacatcaaccggtggcgcattgccacgaagcag

SEQ ID NO: 76 - DNA, 10 bp, synthetic construct
  misc_feature 1..10: note = Synthetic sequence, Emx1 deletion mutant
  source 1..10: mol_type = other DNA
ctgccctacc

SEQ ID NO: 77 - DNA, 10 bp, synthetic construct
  misc_feature 1..10: note = Synthetic sequence, Emx1 deletion mutant
  source 1..10: mol_type = other DNA
gcccacctgg

SEQ ID NO: 78 - DNA, 10 bp, synthetic construct
  misc_feature 1..10: note = Synthetic sequence, Emx1 deletion mutant
  source 1..10: mol_type = other DNA
gagctgagga

SEQ ID NO: 79 - DNA, 10 bp, synthetic construct
  misc_feature 1..10: note = Synthetic sequence, Emx1 deletion mutant
  source 1..10: mol_type = other DNA
accctcagct

SEQ ID NO: 80 - DNA, 10 bp, synthetic construct
  misc_feature 1..10: note = Synthetic sequence, Emx1 deletion mutant
  source 1..10: mol_type = other DNA
tgaggtctca

SEQ ID NO: 81 - DNA, 10 bp, synthetic construct
  misc_feature 1..10: note = Synthetic sequence, Emx1 deletion mutant
  source 1..10: mol_type = other DNA
ggactaggcc

SEQ ID NO: 82 - DNA, 10 bp, synthetic construct
  misc_feature 1..10: note = Synthetic sequence, Emx1 deletion mutant
  source 1..10: mol_type = other DNA
ggactaagaa

SEQ ID NO: 83 - DNA, 17 bp, synthetic construct
  misc_feature 1..17: note = Synthetic sequence, Emx1 deletion mutant
  source 1..17: mol_type = other DNA
tgatgcaggccaatggg

(19) United States
(12) Patent Application Publication
(10) Pub. No.: US 2024/0124898 A1
(43) Pub. Date: Apr. 18, 2024

(54) METHOD FOR PRODUCING DNA-EDITED EUKARYOTIC CELL, AND KIT USED IN THE SAME

(71) Applicant: OSAKA UNIVERSITY, Osaka (JP)

(72) Inventors: Tomoji MASHIMO, Osaka (JP); Junji TAKEDA, Osaka (JP); Hiroyuki MORISAKA, Osaka (JP); Kazuto YOSHIMI, Osaka (JP)

(73) Assignee: OSAKA UNIVERSITY, Osaka (JP)

(21) Appl. No.: 18/467,297

(22) Filed: Sep. 14, 2023

Related U.S. Application Data
(63) Continuation of application No. 16/611,308, filed on Nov. 6, 2019, now Pat. No. 11,807,869.

(30) Foreign Application Priority Data
Jun. 8, 2017 (JP) 2017-113747

(51) Int. Cl. C12N 15/90; A61K 48/00; C12N 5/14; C12N 5/16; C12N 9/22; C12N 15/85

(52) U.S. Cl. CPC C12N 15/907; A61K 48/00; C12N 5/14; C12N 5/16; C12N 9/22; C12N 15/85; C12N 15/8509; C12N 2310/20; C12N 2800/22; C12N 2800/80

(57) ABSTRACT

A CRISPR-Cas3 system was successfully established in a eukaryotic cell.

### CROSS REFERENCE TO RELATED APPLICATIONS

This application is a Continuation of U.S. application Ser. No. 16/611,308 filed on Nov. 6, 2019 (allowed), which is a National Stage of International Application No. PCT/JP2018/022066 filed on Jun. 8, 2018, claiming priority based on Japanese Patent Application No. 2017-113747 filed on Jun. 8, 2017.

### INCORPORATION BY REFERENCE OF SEQUENCE LISTING

The content of the electronically submitted sequence listing, file name: Q291086SEQ_LIST_ST26_AS FILED.xml; size: 193,047 bytes; and date of creation: Sep. 14, 2023, is incorporated herein by reference in its entirety.

### TECHNICAL FIELD

The present invention relates to a method for producing DNA-edited eukaryotic cells, animals, and plants, and to a kit used in the method.

### BACKGROUND ART

Bacteria and archaea have adaptive immune mechanisms which specifically recognize and eliminate organisms such as foreign phages that intend to intrude from the outside. These systems, called the CRISPR-Cas systems, first introduce genomic information on the foreign organisms into the self genome (adaptation) Then, when the same foreign organisms intend to intrude again, the systems cleave and eliminate the foreign genomes by using the complementarity of the information introduced in the self genome and the genome sequence (interference).

Recently, genome editing (DNA editing) techniques using the above CRISPR-Cas systems as “DNA editing tools” have been developed (NPL 1).

The CRISPR-Cas systems are roughly divided into “Class 1” and “Class 2,” in which effectors working in the process of cleaving DNA are composed of multiple Cas and a single Cas, respectively. Among other things, as the Class 1 CRISPR-Cas systems, “type I” involving Cas3 and Cascade complexes (meaning Cascade-crRNA complexes, and the same applies below) is widely known. As the Class 2 CRISPR-Cas systems, “type II” involving Cas9 is widely known (hereinafter, regarding the CRISPR-Cas systems, “Class 1 type I” and “Class 2 type II” may be simply referred to as “type I” and “type II,” respectively). In addition, what have been widely used in the conventional DNA editing techniques are the Class 2 CRISPR-Cas systems involving Cas9 (which hereinafter may be referred to as the “CRISPR-Cas9 systems”). For example, NPL 1 reports a Class 2 CRISPR-Cas system which cleaves DNA using Cas9.

On the other hand, for the Class 1 CRISPR-Cas systems, which cleave DNA using Cas3 and Cascade complexes (which hereinafter may also be referred to as the “CRISPR-Cas3 systems”), no successful example of genomic editing has been reported in eukaryotic cells despite a lot of effort. For example, NPL 2 and NPL 3 reported that simple use of a CRISPR-Cas3 system made it possible to completely degrade target DNA in a cell-free system and selectively remove specific E. coli strains. However, these do not mean the success of genome editing, nor have they been demonstrated at all in eukaryotic cells. In addition, PTL 1 proposes to perform genome editing using the FokI nuclease in place of Cas3 in eukaryotic cells (Example 7, FIG. 7, and FIG. 11) because the CRISPR-Cas3 systems degrade target DNA in E. coli by helicase activity and exonuclease activity of Cas3 (Example 5 and FIG. 6). Moreover, PTL 2 proposes deletion of cas3 and repurposing for programmable gene repression by use of inactivated Cas3 (Cas3′ and Cas3″) (for example, Example 15 and claim 4(e)) because the CRISPR-Cas3 systems degrade target DNA in E. coli (FIGS. 4A-4D).

### CITATION LIST

### Patent Literature

[PTL 1] Published Japanese Translation of PCT International Application No. 2015-503535
[PTL 2] Published Japanese Translation of PCT International Application No. 2017-512481

### Non Patent Literature

[NPL 1] Jinek M et al. (2012) A Programmable Dual-RNA Guided DNA Endonuclease in Adaptive Bacterial Immunity, Science, Vol. 337 (Issue 6096), pp. 816-821
[NPL 2] Mulepati S & Bailey S (2013) In Vitro Reconstitution of an Escherichia coli RNA-guided Immune System Reveals Unidirectional, ATP-dependent Degradation of DNA Target, Journal of Biological Chemistry, Vol. 288 (No. 31), pp. 22184-22192
[NPL 3] Ahmed A. Gomaa et al. (2014) Programmable Reomoval of Bacterial Strains by Use of Genome Targeting CRISPR-Cas Systems, mbio.asm.org, Volume 5, Issue 1, e00928-13

### SUMMARY OF INVENTION

### Technical Problem

The present invention has been made in view of the above circumstances, and an object thereof is to establish a CRISPR-Cas3 system in eukaryotic cells.

### Solution to Problem

The present inventors have made earnest studies to achieve the above object, and finally succeeded in establishing a CRISPR-Cas3 system in eukaryotic cells. The most widely used CRISPR-Cas9 system has succeeded in genome editing in various eukaryotic cells, but this system usually uses a mature crRNA as a crRNA. However, it was surprising that, in the CRISPR-Cas3 systems, genome editing was difficult in eukaryotic cells in the case of using a mature crRNA and that efficient genomic editing was possible only by using a pre-crRNA, which usually was not used as a constituent element of a system. That is, in order to make the CRISPR-Cas3 systems function in eukaryotic cells, cleaving of a crRNA by proteins constituting the Cascade was found to be important. The CRISPR-Cas3 systems using this pre-crRNA were widely applicable not only to the type I-E system but also to the type I-F and type I-G systems. Moreover, addition of a nuclear localization signal, particularly a bipartite nuclear localization signal to Cas3 made it possible to further improve the genome editing efficiency for the CRISPR-Cas3 systems in eukaryotic cells. Furthermore, the present inventors have found that the CRISPR-Cas3 systems, unlike the CRISPR-Cas9 systems can cause a large deletion in a region containing a PAM sequence or in an upstream region thereof. These findings have led to the completion of the present invention.

Specifically, the present invention relates to a CRISPR-Cas3 system in eukaryotic cells, and more specifically to the following invention.

[1] A method for producing a DNA-edited eukaryotic cell, comprising: introducing a CRISPR-Cas3 system into a eukaryotic cell, wherein the CRISPR-Cas3 system includes the following (A) to (C).
(A) a Cas3 protein, a polynucleotide encoding the protein, or an expression vector containing the polynucleotide,
(B) a Cascade protein, a polynucleotide encoding the protein, or an expression vector containing the polynucleotide, and
(C) a crRNA, a polynucleotide encoding the crRNA, or an expression vector containing the polynucleotide.

[2] A method for producing a DNA-edited animal (excluding a human) or plant, comprising: introducing a CRISPR-Cas3 system into an animal (excluding a human) or plant, wherein the CRISPR-Cas3 system includes the following (A) to (C).
(A) a Cas3 protein, a polynucleotide encoding the protein, or an expression vector containing the polynucleotide,
(B) a Cascade protein, a polynucleotide encoding the protein, or an expression vector containing the polynucleotide, and
(C) a crRNA, a polynucleotide encoding the crRNA, or an expression vector containing the polynucleotide.

[3] The method according to [1] or [2], further comprising cleaving the crRNA with a protein constituting the Cascade protein after introducing the CRISPR-Cas3 system into the eukaryotic cell.

[4] The method according to [1] or [2], wherein the crRNA is a pre-crRNA.

[5] The method according to any one of [1] to [4], wherein a nuclear localization signal is added to the Cas3 protein and/or the Cascade protein.

[6] The method according to [5], wherein the nuclear localization signal is a bipartite nuclear localization signal.

[7] A kit for use in the method according to any one of [1] to [6], the kit comprising the following (A) and (B).
(A) a Cas3 protein, a polynucleotide encoding the protein, or an expression vector containing the polynucleotide and
(B) a Cascade protein, a polynucleotide encoding the protein, or an expression vector containing the polynucleotide.

[8] The kit according to [7], further comprising a crRNA, a polynucleotide encoding the crRNA, or an expression vector containing the polynucleotide.

[9] The kit according to [8], wherein the crRNA is a pre-crRNA.

[10] The kit according to any one of [7] to [9], wherein a nuclear localization signal is added to the Cas3 protein and/or the Cascade protein.

[11] The kit according to [10], wherein the nuclear localization signal is a bipartite nuclear localization signal.

Note that in the present specification, the term “polynucleotide” intends a polymer of nucleotides and is used synonymously with the term “gene,” “nucleic acid,” or “nucleic acid molecule.” The polynucleotide may also be present in the form of DNA (for example, cDNA or genomic DNA) or in the form of RNA (for example, mRNA). Also, the term “protein” is used synonymously with “peptide” or “polypeptide.”

### Advantageous Effects of Invention

Use of the CRISPR-Cas3 system of the present invention made it possible to edit DNA in eukaryotic cells.

### BRIEF DESCRIPTION OF DRAWINGS

FIG. 1 is the results of SSA assay measuring cleavage activity against exogenous DNA.

FIG. 2 is a schematic diagram showing the position of the target sequence in the CCR5 gene (SEQ ID NO: 63).

FIG. 3A is a diagram showing the CCR5 gene (clone 1, SEQ ID NO: 64) in which a part of the nucleic acid sequence has been deleted by the CRISPR-Cas3 system.

FIG. 3B is a diagram showing the CCR5 gene (clone 2, SEQ ID NO: 65) in which a part of the nucleic acid sequence has been deleted by the CRISPR-Cas3 system.

FIG. 3C is a diagram showing the CCR5 gene (clone 3, SEQ ID NO: 66) in which a part of the nucleic acid sequence has been deleted by the CRISPR-Cas3 system.

FIG. 3D is a diagram showing the CCR5 gene (clone 4, SEQ ID NO: 67) in which a part of the nucleic acid sequence has been deleted by the CRISPR-Cas3 system.

FIG. 4A is a schematic diagram showing the structure of a Cascade plasmid.

FIG. 4B is a schematic diagram showing the structure of a Cas3 plasmid.

FIG. 4C is a schematic diagram showing the structure of a pre-crRNA plasmid.

FIG. 4D is a schematic diagram showing the structure of a reporter vector (including the target sequence).

FIG. 5 is a schematic diagram showing the position of the target sequence in the EMX1 gene (SEQ ID NO: 68).

FIG. 6A is a diagram showing the EMX1 gene (clone 1, SEQ ID NO: 69) deleted in part of the nucleic acid sequence by the CRISPR-Cas3 system.

FIG. 6B is a diagram showing the EMX1 gene (clone 2, SEQ ID NO: 70) deleted in part of the nucleic acid sequence by the CRISPR-Cas3 system.

FIG. 7 is a schematic diagram showing the structure of a Cas3/Cascade plasmid added with bpNLSs.

FIG. 8 is a schematic diagram showing the structure of a Cascade (2A) plasmid.

FIG. 9 is the results of SSA assay measuring cleavage activity against exogenous DNA

FIG. 10A is a diagram showing the structures of the pre-crRNAs (LRSR and RSR, SEQ ID NOs: 71 and 72 respectively) and the mature crRNA (SEQ ID NO: 73) used in Examples. In the figure, the underlines show the 5′ handle (Cas5 handle), and the double underlines shows the 3′ handle (Cas6 handle).

FIG. 10B is a diagram showing the results of SSA assay using the pre-crRNAs (LRSR and RSR) and the mature crRNA.

FIG. 11 shows the results of SSA assay using a single NLS or two NLSs (bpNLS) in a plasmid for the expression of the Cas3/Cascade gene.

FIG. 12 is a diagram showing the effects of the PAM sequence on the DNA cleavage activity of the CRISPR-Cas3 system.

FIG. 13 is a diagram showing the effects of a single mismatch of the spacer on the DNA cleavage activity of the CRISPR-Cas3 system.

FIG. 14 is a diagram showing the effects of Cas3 mutation in HD nuclease domain (H74A), SF2 helicase domain motif 1 (K320A), and motif 3 (S483/T485A).

FIG. 15 shows a comparison of the DNA cleavage activity of the type I-E, type I-F, and type I-G CRISPR-Cas3 systems.

FIG. 16 is a diagram showing the magnitude of deletion by the CRISPR-Cas3 system detected by the sequencing of a TA cloning sample of a PCR product.

FIG. 17 is a diagram showing the position of deletion by the CRISPR-Cas3 system detected by a mass-processing sequencing of a TA clone (n=49).

FIG. 18A is a diagram showing the number for each deletion size detected by the CRISPR-Cas3 system using a microarray-based capture sequence of 1000 kb or more around the targeted EMX1 locus.

FIG. 18B is a diagram showing the number for each deletion size detected by the CRISPR-Cas3 system using a microarray-based capture sequence of 1000 kb or more around the targeted CCR5 locus.

### DESCRIPTION OF EMBODIMENTS

[1] Method for Producing DNA-Edited Eukaryotic Cells, Animals, and Plants

A method of the present invention comprises introducing a CRISPR-Cas3 system into a eukaryotic cell, wherein the CRISPR-Cas3 system includes the following (A) to (C).
(A) a Cas3 protein, a polynucleotide encoding the protein, or an expression vector containing the polynucleotide,
(B) a Cascade protein, a polynucleotide encoding the protein, or an expression vector containing the polynucleotide, and
(C) a crRNA, a polynucleotide encoding the crRNA, or an expression vector containing the polynucleotide

The Class 1 CRISPR-Cas systems are classified into type I and type III, and depending on the types of proteins constituting the Cascade (hereinafter simply referred to as the “Cascade” or the “Cascade proteins”), type I is further classified into six types of type I-A, type I-B, type I-C, type I-D, type I-E, and type I-F as well as type I-G, a subtype of type I-B (for example, see [van der Oost J et al. (2014) Unravelling the structural and mechanistic basis of CRISPR-Cas systems, Nature Reviews Microbiologym, Vol. 12 (No. 7), pp. 479-492] and [Jackson R N et al. (2014) Fitting CRISPR-associated Cas3 into the Helicase Family Tree, Current Opinion in Structural Biology, Vol. 24, pp. 106-114]).

The type I CRISPR-Cas systems have the function of cleaving DNA by cooperation of Cas3 (protein having nuclease activity and helicase activity), Cascade, and crRNAs. They are referred to as the “CRISPR-Cas3 system” in the present invention because Cas3 is used as a nuclease.

Use of the CRISPR-Cas3 system of the present invention makes it possible to obtain, for example, the following advantages.

First, the crRNA used in the CRISPR-Cas3 system generally recognizes a target sequence of 32 to 37 bases (Ming Li et al., Nucleic Acids Res. 2017 May 5; 45(8): 4642-4654). On the other hand, the crRNA used in the CRISPR-Cas9 system generally recognizes a target sequence of 18 to 24 bases. Therefore, it is considered that the CRISPR-Cas3 system can recognize target sequences more accurately than the CRISPR-Cas9 system.

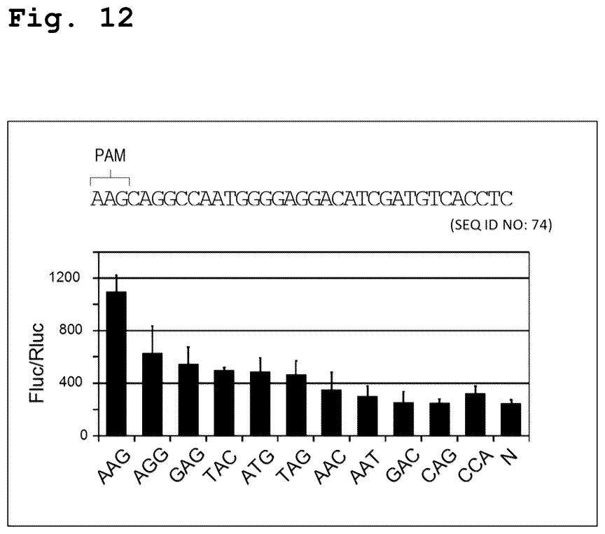

In addition, the PAM sequence of the Class 2 type II system, the CRISPR-Cas9 system, is “NGG (N is an arbitrary base)” adjacent to the 3′ side of the target sequence. Also, the PAM sequence of the Class 2 type V system, the CRISPR-Cpf1 system, is “AA” adjacent to the 5′ side of the target sequence. On the other hand, the PAM sequence of the CRISPR-Cas3 system of the present invention is “AAG” adjacent to the 5′ side of the target sequence or a nucleic acid sequence similar to that (for example, “AGG,” “GAG,” “TAC,” “ATG,” “TAG,” and the like) (FIG. 12). Thus, it is considered that, by using the CRISPR-Cas3 system of the present invention, regions which cannot be recognized by conventional methods can be subjected to DNA editing.

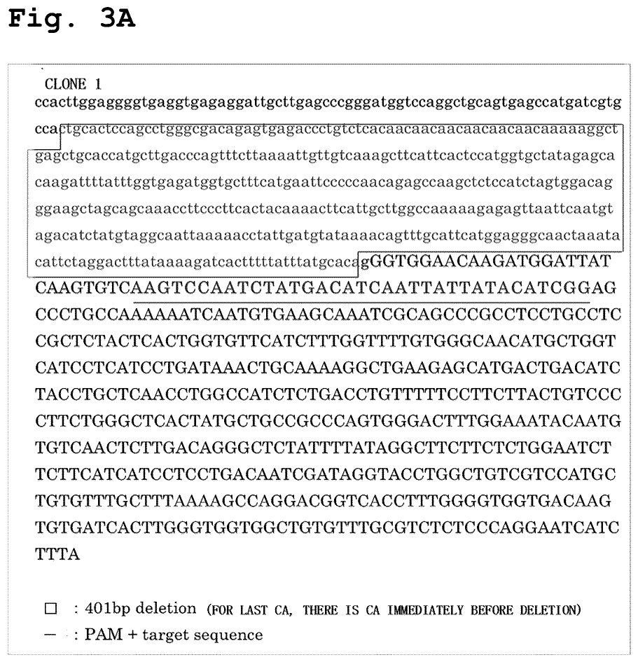

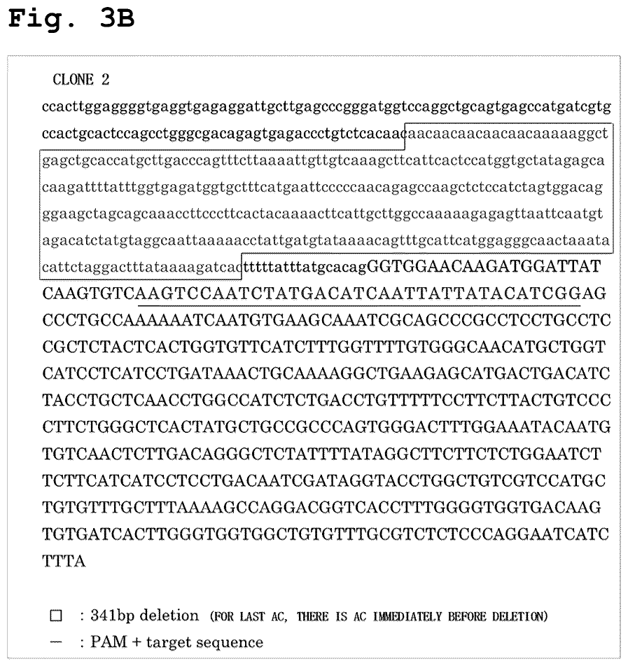

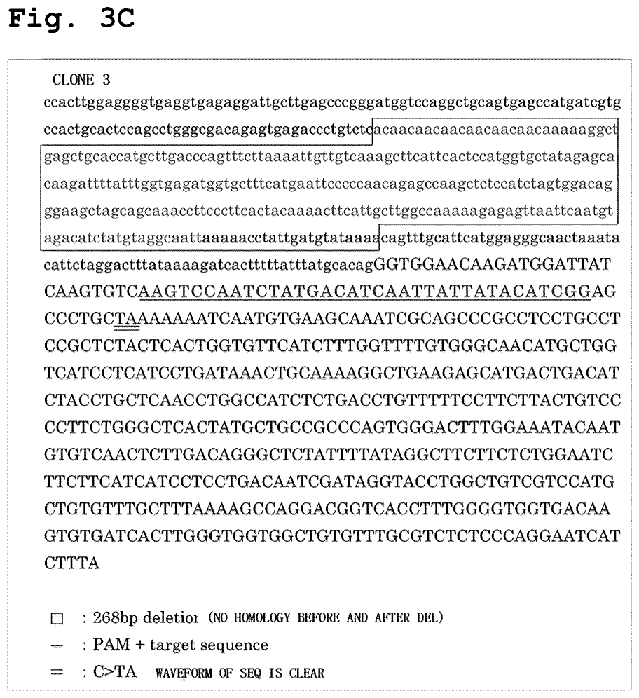

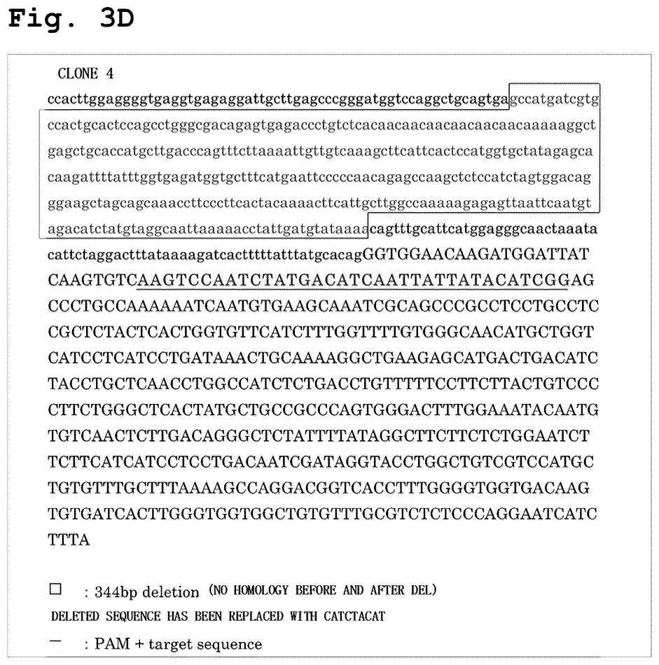

Furthermore, unlike the above Class 2 CRISPR-Cas systems, the CRISPR-Cas3 system causes DNA cleavages at multiple locations. Therefore, use of the CRISPR-Cas3 system of the present invention makes it possible to generate a wide range of deletion mutations ranging from one hundred to several thousand, and possibly even more bases (FIGS. 3, 6, and 16 to 18). It is considered that this function can be used for knocking out a long genomic region or knocking in long DNA. When performing knock-in, donor DNA is usually used, and the donor DNA is also a molecule constituting the CRISPR-Cas3 system of the present invention.

Note that, when simply described as “Cas3” in the present specification, it means a “Cas3 protein.” The same applies to Cascade proteins.

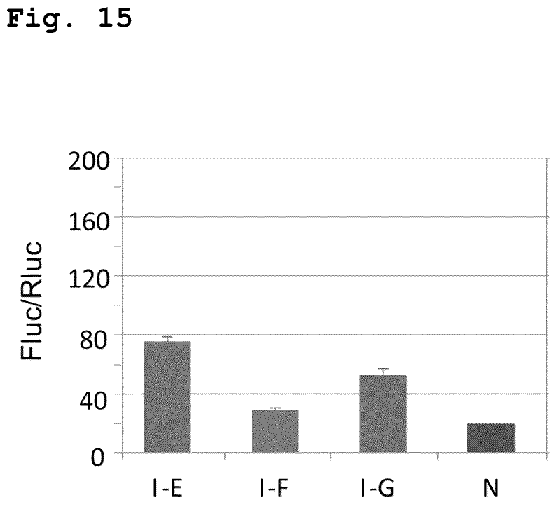

The CRISPR-Cas3 system of the present invention includes all six subtypes of type I. That is, although proteins constituting the CRISPR-Cas3 system may differ slightly in constitution and the like depending on the subtype (for example, the proteins constituting the Cascade are different), the present invention includes all of these proteins. Indeed, in the present example, it was found that genomic editing is possible not only for type I-E but also for type 1-G and type I-F systems (FIG. 15).

The type I-E CRISPR-Cas3 system, which is common among type I CRISPR-Cas3 systems, cleaves DNA when a crRNA cooperates with Cas3 and Cascade (Cse1 (Cas8), Cse2 (Cas11), Cas5, Cas6, and Cas7).

The type I-A system has Cascade constituent elements of Cas8a1, Csa5 (Cas11), Cas5, Cas6, and Cas7, the type I-B has Cascade constituent elements of Cas8b1, Cas5, Cas6, and Cas7, the type I-C has Cascade constituent elements of Cas8c, Cas5, and Cas7, the type I-D has Cascade constituent elements of Cas10d, Csc1 (Cas5), Cas6, and Csc2 (Cas7), the type I-F has Cascade constituent elements of Csy1 (Cas8f), Csy2 (Cas5), Cas6, and Csy3 (Cas7), and the type I-G system has Cascade constituent elements Cst1 (Cas8a1), Cas5, Cas6, and Cst2 (Cas7). In the present invention, Cas3 and Cascade are collectively referred to as the “Cas protein group.”

Hereinafter, the type I-E CRISPR-Cas3 system is described as a representative example. For other types of CRISPR-Cas3 systems, the Cascade constituting the systems may be interpreted as appropriate.

—Cas Protein Group—

In the CRISPR-Cas3 system of the present invention, the Cas protein group can be introduced into eukaryotic cells in the form of a protein, in the form of a polynucleotide encoding the protein, or in the form of an expression vector containing the polynucleotide. When the Cas protein group is introduced into eukaryotic cells in the form of a protein, it is possible to appropriately prepare the amount and the like of each protein, which is excellent from the viewpoint of handling. Moreover, taking into consideration, for example, the efficiency of cleavage in cells, it is also possible to first form a complex of the Cas protein group and then to introduce it to eukaryotic cells.

In the present invention, it is preferable to add a nuclear localization signal to the Cas protein group. The nuclear localization signal can be added to the N-terminus side and/or the C-terminus side of the Cas protein group (5′-end side and/or the 3′-end side of the polynucleotide encoding each Cas protein group). In this way, addition of a nuclear localization signal to the Cas protein group promotes localization to the nucleus in a cell, making it possible to efficiently perform DNA editing as a result.

The above nuclear localization signal is a peptide sequence composed of several to several tens of basic amino acids, and its sequence is not particularly limited as long as proteins are transferred into the nucleus. A specific example of such nuclear localization signal is described in, for example, [Wu J et al. (2009) The Intracellular Mobility of Nuclear Import Receptors and NLS Cargoes, Biophysical journal, Vol. 96 (Issue 9), pp. 3840-3849]. Any nuclear localization signal usually used in the technical field can be used in the present invention.

The nuclear localization signal may be, for example, PKKKRKV (SEQ ID NO: 52) (encoded by the nucleic acid sequence CCCAAGAAGAAGCGGAAGGTG (SEQ ID NO: 53)). When the above nuclear localization signal is used, it is preferable to arrange, for example, a polynucleotide composed of the nucleic acid sequence with SEQ ID NO: 53 on the 5′-end side of the polynucleotide encoding each Cas protein group. In addition, the nuclear localization signal can be, for example, KRTADGSEFESPKKKRKVE (SEQ ID NO: 54) (encoded by the nucleic acid sequence AAGCGGACTGCTGATGGCAGTGAATTTGAGTCCCCAAAGAAGAAGAGAAAGGTG GAA (SEQ ID NO: 55)). When the above nuclear localization signal is used, it is preferable to arrange, for example, polynucleotides composed of the nucleic acid sequences with SEQ ID NO: 55 on both sides of the polynucleotide encoding each Cas protein group (specifically, to use a “bipartite nuclear localization signal (bpNLS)”).

Such modifications are important for allowing the CRISPR-Cas3 system of the present invention to be expressed and to function efficiently in eukaryotic cells, together with the utilization of pre-crRNAs described later.

One preferred embodiment of the Cas protein group used in the present invention is as follows.
Cas3; a protein encoded by a polynucleotide composed of a nucleic acid sequence represented by SEQ ID NO: 1 or SEQ ID NO: 7
Cse1 (Cas8); a protein encoded by a polynucleotide composed of a nucleic acid sequence represented by SEQ ID NO: 2 or SEQ ID NO: 8
Cse2 (Cas11); a protein encoded by a polynucleotide composed of a nucleic acid sequence represented by SEQ ID NO: 3 or SEQ ID NO: 9
Cas5; a protein encoded by a polynucleotide composed of a nucleic acid sequence represented by SEQ ID NO: 4 or SEQ ID NO: 10
Cas6; a protein encoded by a polynucleotide composed of a nucleic acid sequence represented by SEQ ID NO: 5 or SEQ ID NO: 11
Cas7; a protein encoded by a polynucleotide composed of a nucleic acid sequence represented by SEQ ID NO: 6 or SEQ ID NO: 12

The above Cas protein group is (1) a protein obtained by adding PKKKRKV (SEQ ID NO: 52) as a nuclear localization signal to the N-termini of Cas3, Cse1 (Cas8), Cse2 (Cas11), Cas5, Cas6, and Cas7 of wild type E. coli, or (2) a protein obtained by adding KRTADGSEFESPKKKRKVE (SEQ ID NO: 54) as a nuclear localization signal to the N-termini and C-termini of Cas3, Cse1 (Cas8), Cse2 (Cas11), Cas5, Cas6, and Cas7 of wild type E. coli. With proteins having such amino acid sequences, the above Cas protein group can be transferred into the nucleus of a eukaryotic cell. The above Cas protein group transferred to the nucleus in this way cleaves the target DNA. In addition, it is possible to edit target DNA even in a DNA region having a strong structure considered to be difficult in the CRISPAR-Cas9 system (heterochromatin and the like).

Another embodiment of the proteins in the Cas protein group used in the present invention is a protein encoded by a nucleic acid sequence having 90% or more sequence identity with the nucleic acid sequence of the above Cas protein group. Another embodiment of the proteins in the Cas protein group used in the present invention is a protein encoded by a polynucleotide which hybridizes with a polynucleotide composed of a nucleic acid sequence complementary to the nucleic acid sequence of the Cas protein group described above under stringent conditions. Each of the above proteins has DNA cleavage activity when forming a complex with another protein constituting the Cas protein group. The meanings of terms such as “sequence identity” and “stringent conditions” are described later.

—Polynucleotide Encoding Cas Protein Group—

Polynucleotides encoding wild type proteins constituting the type I-E CRISPR-Cas system include polynucleotides modified to be efficiently expressed in eukaryotic cells. That is, it is possible to use a polynucleotide which encodes the Cas protein group and which has been modified. One preferred embodiment of the modification of polynucleotides is modification to a nucleic acid sequence suitable for expression in eukaryotic cells, for example, optimization of a codon to be expressed in eukaryotic cells.

One preferred embodiment of polynucleotides encoding the Cas protein group used in the present invention is as follows
Cas3; a polynucleotide composed of a nucleic acid sequence represented by SEQ ID NO: 1 or SEQ ID NO: 7
Cse1 (Cas8); a polynucleotide composed of a nucleic acid sequence represented by SEQ ID NO: 2 or SEQ ID NO: 8
Cse2 (Cas11); a polynucleotide composed of a nucleic acid sequence represented by SEQ ID NO: 3 or SEQ ID NO: 9
Cas5; a polynucleotide composed of a nucleic acid sequence represented by SEQ ID NO: 4 or SEQ ID NO: 10
Cas6; a polynucleotide composed of a nucleic acid sequence represented by SEQ ID NO: 5 or SEQ ID NO: 11
Cas7; a polynucleotide composed of a nucleic acid sequence represented by SEQ ID NO: 6 or SEQ ID NO: 12

Each of these is a polynucleotide made to function and be expressed in mammalian cells by artificially modifying the nucleic acid sequences encoding the wild type Cas protein group of E. coli (Cas3; SEQ ID NO: 13, Cse1 (Cas8); SEQ ID NO: 14, Cse2 (Cas11); SEQ ID NO: 15, Cas5; SEQ ID NO: 16, Cas6; SEQ ID NO: 17, Cas7; SEQ ID NO: 18).

The above artificial modification of polynucleotides is to modify to a nucleic acid sequence suitable for expression in eukaryotic cells and to add a nuclear localization signal. Modification of a nucleic acid sequence and addition of a nuclear localization signal are as described above. As a result, it can be expected that, for the Cas protein group, the expression level will be sufficiently increased and the functions will be improved.

Another embodiment of polynucleotides encoding the Cas protein group used in the present invention is a polynucleotide formed by modifying a nucleic acid sequence encoding the wild type Cas protein group, composed of a nucleic acid sequence having 90% or more sequence identity with the nucleic acid sequence of the above Cas protein group. Proteins expressed from these polynucleotides have DNA cleavage activity when forming a complex with proteins expressed from other polynucleotides constituting the Cas protein group.

The sequence identity of nucleic acid sequences may be at least 90% or more and more preferably 95% or more (for example, 95%, 96%, 97%, 98%, and 99% or more) in the entire nucleic acid sequence (or the region encoding the site required for the functions of Cse3). It is possible to determine the identity of nucleic acid sequences using a program such as BLASTN (see [Altschul S F (1990) Basic local alignment search tool, Journal of Molecular Biology, Vol. 215 (Issue 3), pp. 403-410]). Examples of the parameters for analyzing nucleic acid sequences by BLASTN include score=100 and word length=12. Specific methods for analysis by BLASTN are known to those skilled in the art. Addition or deletion (gap and the like) may be allowed in order to align the nucleic acid sequences to be compared with the optimal state.

Moreover, “having DNA cleavage activity” is intended to mean the ability to cleave at least one site of a polynucleotide strand.

It is preferable for the CRISPR-Cas3 system of the present invention to cleave DNA by specifically recognizing the target sequence. For example, the dual-Luciferase assay described in Example A-1 makes it possible to know whether or not the CRISPR-Cas3 system specifically recognizes the target sequence.

Another embodiment of polynucleotides encoding the Cas protein group used in the present invention is a polynucleotide which hybridizes with a polynucleotide composed of a nucleic acid sequence complementary to the nucleic acid sequence of the Cas protein group described above under stringent conditions. Proteins expressed from these polynucleotides have DNA cleavage activity when forming a complex with proteins expressed from other polynucleotides constituting the Cas protein group.

Here, the “stringent conditions” refer to the conditions under which two polynucleotide strands form a double-stranded polynucleotide specific for a nucleic acid sequence but does not form a nonspecific double-stranded polynucleotide. The phrase “hybridizes under stringent conditions” can be said in other words as conditions capable of hybridizing in a temperature range from a melting temperature (Tm value) of nucleic acids with high sequence identity (for example, perfectly matched hybrids) to a temperature lower by 15° C., preferably by 10° C., and more preferably by 5° C.

Examples of the stringent conditions are shown as follows. First, two types of polynucleotides are hybridized for 16 to 24 hours at 60 to 68° C. (preferably 65° C. and more preferably 68° C.) in a buffer solution (pH 7.2) composed of 0.25 M Na₂HPO₄, 7% SDS, 1 mM EDTA, and 1×Denhardt's solution. Thereafter, washing is carried out twice for 15 minutes in a buffer solution (pH 7.2) composed of 20 mM Na₂HPO₄, 1% SDS, and 1 mM EDTA at 60 to 68° C. (preferably 65° C. and more preferably 68° C.).

Other examples include the following method. First, prehybridization is carried out overnight at 42° C. in a hybridization solution containing 25% formamide (50% formamide under more severe conditions), 4×SSC (sodium chloride/sodium citrate), 50 mM Hepes (pH 7.0), 10×Denhardt's solution, and 20 μg/mL of denatured salmon sperm DNA. Thereafter, labeled probes are added, and incubation is carried out overnight at 42° C. to hybridize the two kinds of polynucleotides.

Next, washing is carried out under any of the following conditions. Normal condition; 1×SSC and 0.1% SDS are used as washing liquids for washing at about 37° C. Severe condition; 0.5×SSC and 0.1% SDS are used as washing liquids for washing at about 42° C. More severe condition; 0.2×SSC and 0.1% SDS are used as washing liquids for washing at about 65° C.

As the washing conditions for hybridization become more severe, the specificity of hybridization becomes higher. Note that the above combination of conditions SSC, SDS, and temperature is merely illustrative. Stringency similar to the above can be achieved by appropriately combining the above-mentioned elements for determining the stringency of hybridization or other elements (for example, probe concentration, probe length, and hybridization reaction time). This is described in, for example, [Joseph Sambrook & David W. Russell, Molecular cloning: a laboratory manual 3rd Ed., New York: Cold Spring Harbor Laboratory Press, 2001].

—Expression Vector Containing Polynucleotide Encoding Cas Protein Group—

In the present invention, it is possible to use an expression vector for expressing the Cas protein group. Regarding the expression vector, various types of commonly used vectors can be used as a base vector, and it can be appropriately selected depending on the cells for introduction or the introduction method. Specific examples usable include plasmids, phages, cosmids, and the like. The specific type of the vector is not particularly limited, and it suffices to appropriately select a vector which can be expressed in the host cell.

Examples of the expression vectors described above include phage vectors, plasmid vectors, viral vectors, retroviral vectors, chromosome vectors, episomal vectors, virus-derived vectors (bacterial plasmids, bacteriophages, yeast episomes, and the like), yeast chromosomal elements and viruses (baculoviruses, papova viruses, vaccinia viruses, adenoviruses, tripox viruses, pseudorabies viruses, herpes viruses, lentiviruses, retroviruses, and the like), and vectors derived from combinations thereof (cosmids, phagemids, and the like).

Preferably, the expression vector further contains a site for transcription initiation and transcription termination as well as a ribosome binding site in the transcription region. The coding site of the mature transcript in the vector will contain the transcription initiation codon AUG at the beginning and an appropriately located termination codon at the end of the polypeptide to be translated.

In the present invention, the expression vector for expressing the Cas protein group may contain a promoter sequence. The above promoter sequence may be appropriately selected depending on the type of eukaryotic cell serving as a host. In addition, the expression vector may contain a sequence for enhancing transcription from DNA, for example an enhancer sequence. Examples of enhancers include the SV40 enhancer (which is arranged at 100-270 bp downstream of the replication origin), the early promoter enhancer of the cytomegalovirus, and the polyoma enhancer and the adenovirus enhancer arranged downstream of the replication origin. Additionally, the expression vector may contain a sequence for stabilizing a transcribed RNA, for example a poly(A) addition sequence (polyadenylation sequence, polyA). Examples of poly(A) addition sequences include poly(A) addition sequences derived from the growth hormone gene, poly(A) addition sequences derived from the bovine growth hormone gene, poly(A) addition sequences derived from the human growth hormone gene, poly(A) addition sequences from the SV40 virus, and poly(A) additional sequences derived from the human or rabbit β-globin gene.

The number of polynucleotides encoding the Cas protein group to be incorporated into the same vector is not particularly limited as long as it is possible to exhibit the functions of the CRISPR-Cas systems in the host cell into which the expression vector has been introduced. For example, it is possible to make such a design that the polynucleotide encoding the Cas protein group is mounted on vectors of one type (of the same type). Furthermore, it is also possible to make such a design that all or some of the polynucleotide encoding the Cas protein groups is mounted on separate vectors. For example, it is possible to make such a design that the polynucleotide encoding Cascade proteins is mounted on vectors of one type (of the same type) and the polynucleotide encoding Cas3 is mounted on other vectors. It is preferable to use a method for mounting the polynucleotide encoding Cas protein groups on six different types of vectors from the viewpoint of expression efficiency and the like.

Otherwise, multiple polynucleotides encoding the same proteins may be mounted on the same vectors for the purpose of controlling the expression level and the like. For example, it is possible to make such a design that the polynucleotides encoding Cas3 are arranged at two sites of vectors of one type (of the same type).

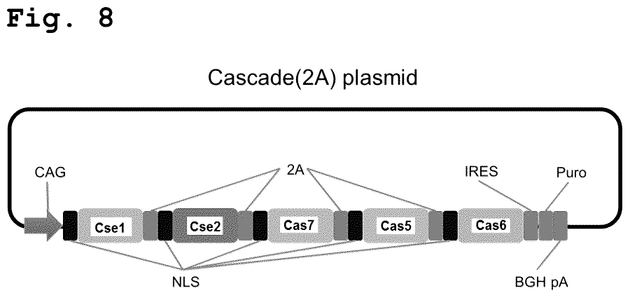

In addition, it is possible to use an expression vector which contains multiple nucleic acid sequences encoding the Cas protein group and which has nucleic acid sequences that are inserted between those multiple nucleic acid sequences and that encode amino acid sequences (2A peptides and the like) to be cleaved by intracellular proteases (for example, see the vector structure of FIG. 8). When polynucleotides having such nucleic acid sequences are transcribed and translated, polypeptide strands linked in the cell are expressed. Subsequently, due to the action of intracellular proteases, the Cas protein groups are separated, become separate proteins, and then form complexes to function. This makes it possible to regulate the amount ratio of Cas protein groups expressed intracellularly. For example, it is predicted that Cas3 and Cse1 (Cas8) will be expressed in equal amounts from an “expression vector containing one nucleic acid sequence encoding Cas3 and one nucleic acid sequence encoding Cse1 (Cas8).” In addition, it is possible to express multiple Cas protein groups with one type of expression vector, which is advantageous in excellence of handling property. On the other hand, the embodiment is usually superior in which the Cas protein groups are expressed by different expression vectors from the viewpoint of high DNA cleavage activity.

It is possible to prepare the expression vectors used in the present invention by known methods. Examples of such methods include the method described in the manual attached to a kit for preparing vectors as well as methods described in various handbooks. An example of a comprehensive handbook is [Joseph Sambrook & David W. Russell, Molecular cloning: a laboratory manual 3rd Ed., New York: Cold Spring Harbor Laboratory Press, 2001].

—Expression Vector Containing crRNA, Polynucleotide Encoding the crRNA, or the Polynucleotide—

The CRISPR-Cas3 system of the present invention includes a crRNA, a polynucleotide encoding the crRNA, or an expression vector containing the polynucleotide for the purpose of targeting to DNA for genome editing.

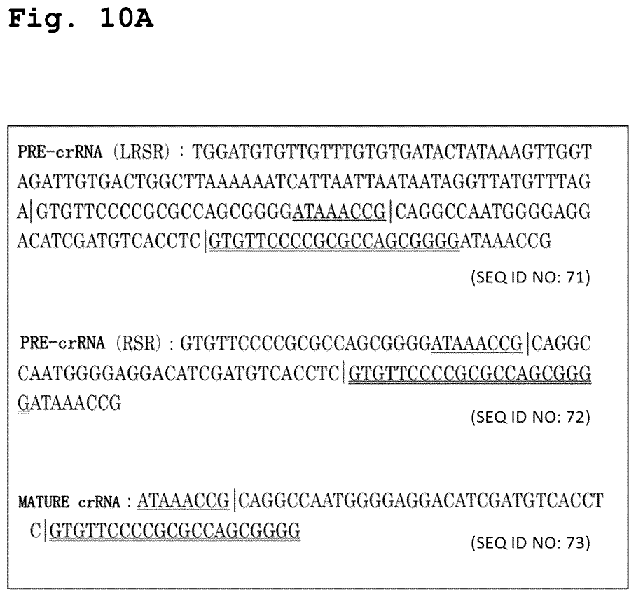

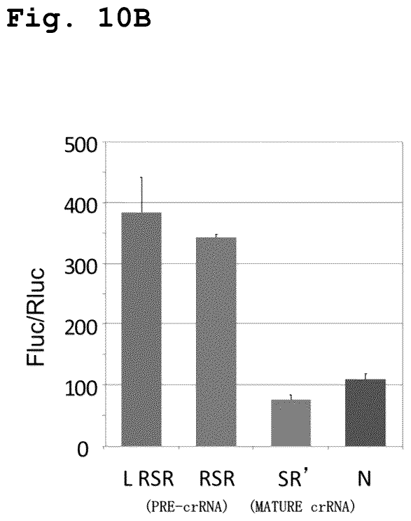

The crRNA is an RNA which forms part of the CRISPR-Cas system and has a nucleic acid sequence complementary to the target sequence. The CRISPR-Cas3 system of the present invention makes it possible with a crRNA to specifically recognize a target sequence and cleave the sequence. In CRISPR-Cas systems typified by the CRISPR-Cas9 system, mature crRNAs have been usually used as cRNAs. However, although the reason is not clear, it was found that use of a mature crRNA is not suitable when the CRISPR-Cas3 system is made to function in eukaryotic cells. Moreover, it was surprisingly found that it is possible to highly efficiently perform genome editing in eukaryotic cells by using a pre-crRNA instead of a mature crRNA. This fact is apparent from a comparative experiment between a mature crRNA and pre-crRNAs (FIG. 10). Therefore, it is particularly preferable to use pre-crRNAs as the crRNAs of the present invention.

The pre-crRNAs used in the present invention typically have the structures of “leader sequence-repeated sequence-spacer sequence-repeated sequence (LRSR structure)” and “repeated sequence-spacer sequence-repeated sequence (RSR structure).” The leader sequence is an AT-rich sequence and functions as a promoter to express a pre-crRNA. The repeated sequence is a sequence repeating with a spacer sequence in between, and the spacer sequence is a sequence designed in the present invention as a sequence complementary to the target DNA (originally it is a sequence derived from a foreign DNA incorporated in the course of adaptation). The pre-crRNA becomes a mature crRNA when cleaved by proteins constituting the Cascade (for example, Cas6 for types I-A, B, and D to E and Cas5 for type I-C).

Typically, the strand length of a leader sequence is 86 bases, and the strand length of a repeated sequence is 29 bases. The strand length of a spacer sequence is, for example, 10 to 60 bases, preferably 20 to 50 bases, more preferably 25 to 40 bases, and typically 32 to 37 bases. Thus, in the case of the LRSR structure, the pre-crRNA used in the present invention has a strand length of, for example, 154 to 204 bases, preferably 164 to 194 bases, more preferably 169 to 184 bases, and typically 176 to 181 bases. In addition, in the case of the RSR structure, the strand length is, for example, 68 to 118 bases, preferably 78 to 108 bases, more preferably 83 to 98 bases, and typically 90 to 95 bases.

In order to make the CRISPR-Cas3 system of the present invention function in eukaryotic cells, it is considered that the process is important by which the repeated sequences of a pre-crRNA are cleaved by the proteins constituting the Cascade. Thus, it should be understood that the above repeated sequences may be shorter or longer than the above strand length as long as such cleavage takes place. Specifically, it can be said that the pre-crRNA is a crRNA formed by adding sequences sufficient for cleavage by proteins constituting the Cascade to both ends of the mature crRNA described below. In this way, a preferred embodiment of the method of the present invention includes the step of cleaving a crRNA with proteins constituting the Cascade after introducing the CRISPR-Cas3 system into eukaryotic cells.

On the other hand, the mature crRNA generated by cleavage of a pre-crRNA has a structure of “5′-handle sequence-spacer sequence-3′-handle sequence.” Typically, the 5′-handle sequence is composed of 8 bases from positions 22 to 29 of the repeated sequence and is held in Cas5. In addition, the 3′-handle sequence is typically composed of 21 bases from positions 1 to 21 in the repeated sequence, forms a stem loop structure with the bases of positions 6 to 21, and is held at Cas6. Thus, the strand length of a mature crRNA is usually 61 to 66 bases. Note that, since there are also mature crRNAs having no 3′-handle sequence depending on the type of the CRISPR-Cas3 system, the strand length is shortened by 21 bases in this case.

Note that the sequence of an RNA may be appropriately designed according to the target sequence for which DNA editing is desired. In addition, it is possible to synthesize an RNA using any method known in the art.

—Eukaryotic Cell—

Examples of “eukaryotic cells” in the present invention include animal cells, plant cells, algae cells, and fungal cells. In addition, examples of animal cells include mammalian cells as well as cells of, for example, fish, birds, reptiles, amphibians, and insects.

Examples of the “animal cells” include cells constituting animal bodies, cells constituting organs/tissues excised from animals, and cultured cells derived from animal tissues. Specific examples include germ cells such as oocytes and sperm; embryonic cells of embryos at various stages (such as 1-cell embryos, 2-cell embryos, 4-cell embryos, 8-cell embryos, 16-cell embryos, and morula embryos); stem cells such as induced pluripotent stem (iPS) cells and embryonic stem (ES) cells; and somatic cells such as fibroblasts, hematopoietic cells, neurons, muscle cells, bone cells, liver cells, pancreatic cells, brain cells, and kidney cells. It is possible to use oocytes before fertilization and after fertilization as the oocytes used for preparing genome-edited animals, preferably oocytes after fertilization, that is, fertilized eggs. Particularly preferably, the fertilized eggs are from pronuclear stage embryos. Oocytes can be thawed and used after freezing.

In the present invention, “mammalian” is a concept including human and non-human mammals. Examples of non-human mammals include cloven-hoofed mammals such as cattle, boars, pigs, sheep, and goats, odd-toed mammals such as horses, rodents such as mice, rats, guinea pigs, hamsters, and squirrels, lagomorphs such as rabbits, and carnivores such as dogs, cats, and ferrets. The non-human mammals described above may be livestock or companion animals (pets), or may be wild animals.

Examples of the “plant cells” include cells of cereals, oil crops, feed crops, fruits, and vegetables. Examples of the “plant cells” include cells constituting plant bodies, cells constituting organs and tissues separated from plants, and cultured cells derived from plant tissues. Examples of organs and tissues of plants include leaves, stems, shoot apexes (growing points), roots, tubers, and calli. Examples of plants include rice, corn, banana, peanut, sunflower, tomato, oilseed rape, tobacco, wheat, barley, potato, soybean, cotton, and carnation as well as propagation materials thereof (for example, seeds, tuberous roots, and tubers).

—DNA Editing—

In the present invention, “editing the DNA of a eukaryotic cell” may be a step in which the DNA of a eukaryotic cell is edited in vivo or in vitro. In addition, “editing the DNA” means the operations exemplified by the following types (including combinations thereof).

Note that, in the present specification, the DNA used in the above context includes not only DNA present in the nucleus of a cell but also exogenous DNA and DNA present other than the nucleus of a cell such as mitochondrial DNA.
1. cleaving the DNA strand at the target site
2. deleting a base of the DNA strand at the target site
3. inserting a base into the DNA strand at the target site
4. replacing a base of the DNA strand at the target site
5. modifying a base of the DNA strand at the target site
6. modulating the transcription of the DNA (gene) at the target site.

One embodiment of the CRISPR-Cas3 system of the present invention uses a protein having an enzymatic activity for modifying the target DNA by a method other than introducing DNA cleavage. This embodiment can be achieved by, for example, fusing Cas3 or Cascade with a heterologous protein having a desired enzymatic activity into a chimeric protein. Thus, “Cas3” and “Cascade” in the present invention also include such fusion proteins. Examples of the enzymatic activity of the protein to be fused include, but not limited to, deaminase activity (for example, cytidine deaminase activity and adenosine deaminase activity), methyl transferase activity, demethylation enzyme activity, DNA repair activity, DNA damage activity, dismutase activity, alkylation activity, depurination activity, oxidation activity, pyrimidine dimer formation activity, integrase activity, transposase activity, recombinase activity, polymerase activity, ligase activity, photoreactivation enzyme activity, and glycosylase activity. In this case, the nuclease activity or the helicase activity of Cas3 is not necessarily required. For this reason, it is possible to use as Cas3 a mutant in which some or all of these activities are deleted (for example, a mutant of D domain H74A (dnCas3), a mutant of K320N of SF2 domain motif 1 (dhCas3), and a double mutant of S483A/T485A of SF2 domain motif 3 (dh2Cas3)). Precise genome editing is possible by replacing bases without causing large deletion at the target site if, for example, a fusion protein of a deaminase and a mutant in which some or all of the nuclease activities of Cas3 have been eliminated is used as a constituent element of the CRISPR-Cas3 system of the present invention. The method for applying a deaminase to the CRISPR-Cas systems is well known (Nishida K. et al., Targeted nucleotide editing using hybrid prokaryotic and vertebrate adaptive immune systems, Science, DOI: 10.1126/science.aaf8729, (2016)), and it suffices to apply the method to the CRISPR-Cas3 system of the present invention.

Another embodiment of the CRISPR-Cas3 system of the present invention regulates gene transcription at the binding site of the present system without DNA cleavage. This embodiment can be achieved by, for example, fusing Cas3 or Cascade with the desired transcription regulating protein into a chimeric protein. Thus, “Cas3” and “Cascade” in the present invention also include such fusion proteins. Examples of the transcription regulating protein include, but not limited to, light inducible transcriptional regulators, small molecule/drug responsive transcriptional regulators, transcription factors, and transcriptional repressors. In this case, the nuclease activity or the helicase activity of Cas3 is not necessarily required. For this reason, it is possible to use as Cas3 a mutant in which some or all of these activities are deleted (for example, a mutant of D domain H74A (dnCas3), a mutant of K320N of SF2 domain motif 1 (dhCas3), and a double mutant of S483A/T485A of SF2 domain motif 3 (dh2Cas3)). Methods for applying a transcription regulating protein to the CRISPR-Cas systems are known to those skilled in the art.

Additionally, in the CRISPR-Cas3 system of the present invention, consider the case of, for example, using a mutant in which some or all of the nuclease activities of Cas3 are deleted. Proteins having other nuclease activities may be fused with Cas3 or Cascade. Such embodiment is included in the present invention.

Besides, in the CRISPR-Cas3 system of the present invention, consider the case of using a mutant in which some or all of the nuclease activities of Cas3 are deleted and using the activities of other proteins in editing DNA. The “DNA cleavage activity” in the present specification is appropriately interpreted as various activities which those proteins have.

Moreover, DNA editing may be performed on DNA contained in a specific cell within an individual. Such DNA editing can be performed on, for example, a specific cell as a target among cells constituting the body of an animal or plant.

No limitation is imposed on the method for introducing the molecules constituting the CRISPR-Cas3 system of the present invention into eukaryotic cells in the form of a polynucleotide or an expression vector containing the polynucleotide. Examples of the method include electroporation, the calcium phosphate method, the liposome method, the DEAE dextran method, the microinjection method, cationic lipid mediated transfection, electroporation, transduction, and infection using virus vectors. Such methods are described in many standard laboratory manuals such as “Leonard G. Davis et al., Basic methods in molecular biology, New York: Elsevier, 1986.”

No limitation is imposed on the method for introducing the molecules of the CRISPR-Cas3 system of the present invention into eukaryotic cells in the form of a protein. Examples thereof include electroporation, cationic lipid mediated transfection, and microinjection.

The DNA editing according to the present invention can be applied to various fields. Application examples include gene therapy, breed improvement, production of transgenic animals or cells, production of useful substances, and life science research.

Known methods can be used as methods for preparing non-human individuals from cells. Germ cells or pluripotent stem cells are usually used in the case of producing non-human individuals from cells of animals. For example, molecules constituting the CRISPR-Cas3 system of the present invention are introduced into an oocyte. The obtained oocyte is then transplanted into the uterus of a female non-human mammal which has been placed in a pseudopregnant state. After that, a litter is obtained. The transplantation can be carried out in a fertilized egg of 1-cell embryo, 2-cell embryo, 4-cell embryo, 8-cell embryo, 16-cell embryo, or morula embryo. If desired, the oocyte can be cultured under suitable conditions until transplantation. Transplantation and culture of the oocyte can be carried out based on a conventionally known method (Nagy A. et al., Manipulating the Mouse Embryo, Cold Spring Harbour, New York: Cold Spring Harbour Laboratory Press, 2003). It is also possible to obtain, from the obtained non-human individual, clones or descendants in which the desired DNA has been edited.

In addition, it has long been known that somatic cells of plants possess differentiation totipotency, and methods for regenerating plants from plant cells of various plants have been established. Therefore, it is possible to obtain a plant in which the desired DNA is knocked in by introducing the molecules constituting the CRISPR-Cas3 system of the present invention into plant cells and regenerating plants from the obtained plant cells. It is also possible to obtain progeny, clones, or propagation materials in which the desired DNA has been edited. As a method of redifferentiating a plant tissue by tissue culture to obtain an individual, it is possible to use a method established in the present technical field (Protocols for Plant Transformation, edited by TABEI Yutaka, Kagaku-Dojin, pp. 340-347 (2012)).

[2] Kit Used in CRISPR-Cas3 System

A kit used in the CRISPR-Cas3 system of the present invention comprises the following (A) and (B).
(A) a Cas3 protein, a polynucleotide encoding the protein, or an expression vector containing the polynucleotide, and
(B) a Cascade protein, a polynucleotide encoding the protein, or an expression vector containing the polynucleotide.

The kit may further comprises a crRNA, a polynucleotide encoding the crRNA, or an expression vector containing the polynucleotide

The constituent elements of the kit of the present invention may be in an embodiment in which all or some of them are mixed, or may be in an embodiment in which each of them is independent.

It is possible to use the kit of the present invention in fields such as pharmaceutical preparations, food, animal husbandry, fishery, industry, bioengineering, and life science research.

Hereinafter, the kit of the present invention is described assuming pharmaceutical preparations (drugs). Note that, in the case of using the above-described kit in fields such as animal husbandry, bioengineering, and life science research, the kit can be used by appropriately interpreting the following explanation based on common technical knowledge in those fields.

It is possible to prepare a pharmaceutical preparation for editing DNA of animal cells including humans by usual methods using the CRISPR-Cas3 system of the present invention. More specifically, the pharmaceutical preparation can be prepared by formulating the molecules constituting the CRISPR-Cas3 system of the present invention with, for example, a pharmaceutical preparation additive.

Here, the “pharmaceutical preparation additive” means a substance other than the active ingredients contained in the pharmaceutical preparation. The pharmaceutical preparation additive is a substance contained in a pharmaceutical preparation for the purpose of facilitating formulation, stabilizing the quality, enhancing the utility, and the like. Examples of the pharmaceutical preparation additive described above can include excipients, binders, disintegrants, lubricants, fluidizers (solid antistatic agents), colorants, capsule coats, coating agents, plasticizers, taste-making agents, sweeteners, flavoring agents, solvents, dissolution assisting agents, emulsifiers, suspending agents (pressure sensitive adhesives), thickeners, pH adjusters (acidifiers, alkalizers, and buffers), humectants (solubilizers), antibacterial preservatives, chelating agents, suppository bases, ointment bases, curing agents, softeners, medical water, propellants, stabilizers, and preservatives. These pharmaceutical preparation additives can readily be selected by those skilled in the art according to the intended dosage form and route of administration as well as standard pharmaceutical practice.

In addition, the pharmaceutical preparation for editing DNA of animal cells using the CRISPR-Cas3 system of the present invention may contain additional active ingredients. The additional active ingredients are not particularly limited and can be appropriately designed by those skilled in the art.

Specific examples of the active ingredients and pharmaceutical preparation additives described above can be learned according to the standards established by, for example, the US Food and Drug Administration (FDA), the European Medicines Authority (EMA), the Japanese Ministry of Health, Labor and Welfare.

Examples of methods for delivering a pharmaceutical preparation to the desired cells include methods using virus vectors targeting the cells (adenovirus vectors, adeno-associated virus vectors, lentivirus vectors, Sendai virus vectors, and the like) or antibodies specifically recognizing the cells. The pharmaceutical preparation can take any dosage form depending on the purpose. Also, the above pharmaceutical preparation is properly prescribed by doctors or medical professionals.

The kit of the present invention preferably further includes an instruction manual.

### EXAMPLES

Hereinafter, the present invention is described in more detail with reference to Examples, but the present invention is not limited only to the following examples.

A. Establishment of CRISPR-Cas3 System in Eukaryotic Cell

[Material and Method]

[1] Preparation of Reporter Vectors Containing Target Sequences

The target sequences were the sequence derived from the human CCR5 gene (SEQ ID NO: 19) and the spacer sequence of E. coli CRISPR (SEQ ID NO: 22).

For the purpose of inserting the target sequences into the vectors, a synthetic polynucleotide (SEQ ID NO: 20) containing the target sequence derived from the human CCR5 gene (SEQ ID NO: 19) and a synthetic polynucleotide (SEQ ID NO: 21) containing a sequence complementary to the above target sequence (SEQ ID NO: 19) were prepared. Similarly, a synthetic polynucleotide (SEQ ID NO: 23) containing the target sequence derived from the spacer sequence of E. coli CRISPR (SEQ ID NO: 22) and a synthetic polynucleotide (SEQ ID NO: 24) containing a sequence complementary to the above target sequence (SEQ ID NO: 22) were prepared. All of the above synthetic polynucleotides were obtained from Hokkaido System Science Co., Ltd.

The above polynucleotides were inserted into the reporter vectors by the method described in [Sakuma T et al. (2013) Efficient TALEN construction and evaluation methods for human cell and animal applications, Genes to Cells, Vol. 18 (Issue 4), pp. 315-326]. The outline is as follows. First, polynucleotides having sequences complementary to each other (the polynucleotide of SEQ ID NO: 20 and the polynucleotide of SEQ ID NO: 21; the polynucleotide of SEQ ID NO: 23 and the polynucleotide of SEQ ID NO: 24) were heated at 95° C. for 5 minutes, and then cooled to room temperature and hybridized. A block incubator (BI-515A, Astec) was used for the above step. Next, the polynucleotide hybridized to form a double-stranded structure was inserted into the base vector to prepare a reporter vector.

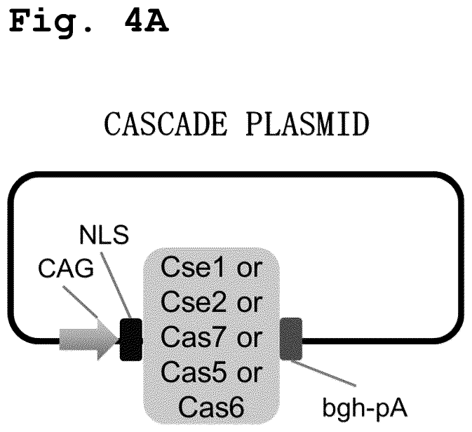

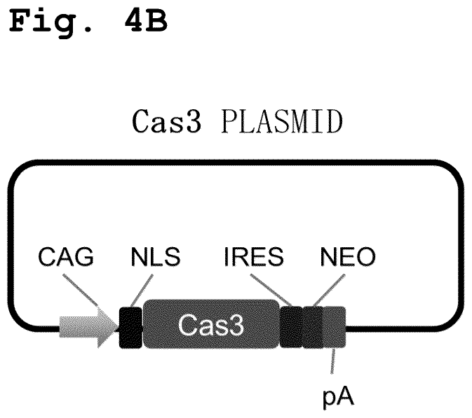

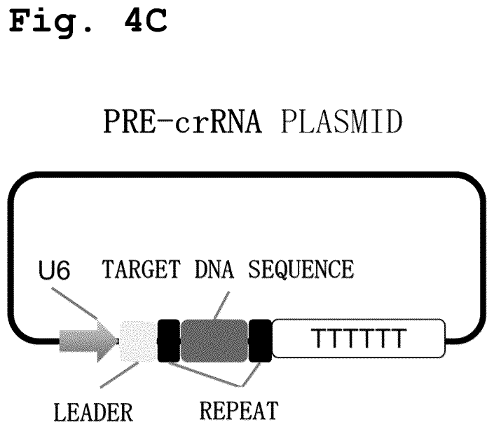

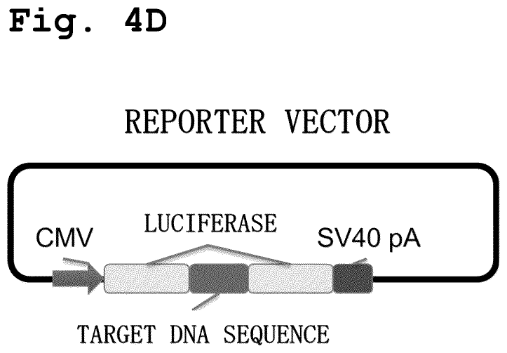

The sequences of the prepared reporter vectors are shown at SEQ ID NO: 31 (reporter vector containing the target sequence derived from the human CCR5 gene) and SEQ ID NO: 32 (reporter vector containing the target sequence derived from the spacer sequence of E. coli CRISPR). In addition, the structure of reporter vector is shown in FIG. 4D.

[2] Preparation of Cse1 (Cas8), Cse2 (Cas11), Cas5, Cas6, Cas7, and crRNA Expression Vectors

[Amplification and Preparation of Inserts]

Consider polynucleotides having modified nucleic acid sequences encoding Cse1 (Cas8), Cse2 (Cas11), Cas5, Cas6, and Cas7 (with SEQ ID NO: 2, SEQ ID NO: 3, SEQ ID NO: 4, SEQ ID NO: 5, and SEQ ID NO: 6, respectively). First, production of polynucleotides linked in the order of SEQ ID NO: 2-SEQ ID NO: 3-SEQ ID NO: 6-SEQ ID NO: 4-SEQ ID NO: 5 (polynucleotides having linked nucleic acid sequences for encoding Cse1 (Cas8)-Cse2 (Cas11)-Cas7-Cas5-Cas6 in this order) was outsourced to GenScript Corporation for purchase. The nucleic acid sequences encoding the proteins of Cse1 (Cas8)-Cse2 (Cas11)-Cas7-Cas5-Cas6 were linked with 2A peptides (amino acid sequence: GSGATNFSLLKQAGDVEENPGP (SEQ ID NO: 58)).

Note that the nucleic acid sequences encoding the 2A peptides were slightly different depending on the Cas protein linkage sites and were as follows.

The sequence between Cse1 (Cas8) and Cse2 (Cas11):
(SEQ ID NO: 59)
GGAAGCGGAGCAACCAACTTCAGCCTGCTGAAGCAGGCCGGCGATGTGGA
GGAGAATCCAGGCCCC.
The sequence between Cse2 (Cas11)and Cas7:
(SEQ ID NO: 60)
GGCTCCGGCGCCACCAATTTTTCTCTGCTGAAGCAGGCAGGCGATGTGGA
GGAGAACCCAGGACCT.
The sequence between Cas7 and Cas5:
(SEQ ID NO: 61)
GGATCTGGAGCCACCAATTTCAGCCTGCTGAAGCAAGCAGGCGACGTGGA
AGAAAACCCAGGACCA.
The sequence between Cas5 and Cas6:
(SEQ ID NO: 62)
GGATCTGGGGCTACTAATTTTTCTCTGCTGAAGCAAGCCGGCGACGTGGA
AGAGAATCCAGGACCG.

Next, each of the polynucleotides was amplified under the PCR conditions (primer and time course) in the following table. For PCR, 2720 Thermal cycler (applied biosystems) was used.

TABLE 1

| Primer | Sequence | Timecourse |
|---|---|---|
| Cse1-U | GCAAAGAATTCAGAT | 98° C. (10 Sec) → |
| | CTCCACCATGCCTAA | 68° C. (1 Min) |
| | GAAGAAGAGAAAAGT | 35 Cycles |
| | GAACCTGCTGATTGA | |
| | C | |
| | (SEQ ID NO: 36) | |
| Cse1-L | TCATCGATGCATCTC | |
| | GAGTTATCCATTAGA | |
| | AGGTCCTCCCTGTGG | |
| | CTTC | |
| | (SEQ ID NO: 37) | |
| Cse2-U | GCAAAGAATTCAGAT | 98° C. (10 Sec) → |
| | CTCCACCATGCCCAA | 68° C. (1 Min) |
| | GAAGAAGCGGAAGGT | 35 Cycles |
| | GGCCGATGAGATCGA | |
| | C | |
| | (SEQ ID NO: 38) | |
| Cse2-L | TCATCGATGCATCTC | |
| | GAGTTAGGCGTTCTT | |
| | ATTTGTGGTCAGCAC | |
| | GAAG | |
| | (SEQ ID NO: 39) | |
| Cas5-U | GCAAAGAATTCAGAT | |
| | CTCCACCATGCCCAA | |
| | GAAGAAGCGGAAGGT | |
| | GTCCAATTTCATCAA | |
| | C | |
| | (SEQ ID NO: 40) | |
| Cas5-L | TCATCGATGCATCTC | |
| | GAGTTATGCCTCTCC | |
| | ATTGTTCCGCACCCA | |
| | GCTC | |
| | (SEQ ID NO: 41) | |
| Cas6-U | GCAAAGAATTCAGAT | |
| | CTCCACCATGCCCAA | |
| | GAAGAAGCGGAAAGT | |
| | GTACCTGAGCAAAGT | |
| | G | |
| | (SEQ ID NO: 42) | |
| Cas6-L | TCATCGATGCATCTC | |
| | GAGTTACAGAGGTGC | |
| | CAGTGACAGCAGCCC | |
| | AC | |
| | (SEQ ID NO: 43) | |
| Cas7-U | GCAAAGAATTCAGAT | 98° C. (10 Sec) → |
| | CTCCACCATGCCCAA | 68° C. (1 Min 40 Sec) |
| | GAAGAAGCGGAAGGT | 35 Cycles |
| | GCGCTCCTACCTGAT | |
| | C | |
| | (SEQ ID NO: 44) | |
| Cas7-L | TCATCGATGCATCTC | |
| | GAGTTACTGGCTCAC | |
| | GTCCATTCCTCCCTT | |
| | GATC | |
| | (SEQ ID NO: 45) | |

Polynucleotides having the following complementary sequences were obtained as polynucleotides having nucleic acid sequences for expressing crRNA.
1. polynucleotides for expressing crRNA corresponding to the sequence derived from the human CCR5 gene (SEQ ID NOs: 25 and 26, obtained from Hokkaido System Science Co., Ltd.)
2. polynucleotides for expressing crRNA corresponding to the spacer sequence of E. coli CRISPR (SEQ ID NOs: 27 and 28, obtained from Hokkaido System Science Co., Ltd.)
3. polynucleotides for expressing crRNA corresponding to the sequence derived from the human EMX1 gene (SEQ ID NOs: 29 and 30, obtained from Pharmac).

[Ligation and Transformation]

As a substrate plasmid, pPB-CAG-EBNXN (supplied from Sanger Center) was used. In NEB buffer, 1.6 μg of the substrate plasmid, 1 μl of restriction enzyme BglII (New England Biolabs), and 0.5 μl of XhoI (New England Biolabs) were mixed and reacted at 37° C. for 2 hours. The cleaved substrate plasmids were purified with Gel extraction kit (Qiagen).

The substrate plasmids thus prepared and the above inserts were ligated with a Gibson Assembly system. Ligation was carried out in accordance with the protocol of the Gibson Assembly system with the ratio of the substrate plasmids to the inserts being 1:1 (at 50° C. for 25 minutes, total volume of the reaction solution: 8 μL).

Subsequently, 6 μL of a solution of the plasmids obtained above (ligation reaction solution) and competent cells (prepared by Takeda Laboratory) were used to perform transformation in accordance with the usual method.

Thereafter, plasmid vectors were purified from the transformed E. coli by the alkaline prep method. Briefly, the plasmid vectors were recovered using QIAPREP SPIN MINIPREP™ Kit (Qiagen), and the recovered plasmid vectors were purified by the ethanol precipitation method and then adjusted to have a concentration of 1 μg/μL in a TE buffer solution.

The structure of each plasmid vector is shown in FIGS. 4 (a) to 4 (c). In addition, the nucleic acid sequences of pre-crRNA expression vectors are shown at SEQ ID NO: 33 (expression vector for expressing crRNA corresponding to the sequence derived from the human CCR5 gene), SEQ ID NO: 34 (expression vector for expressing crRNA corresponding to the spacer sequence of E. coli CRISPR), and SEQ ID NO: 35 (expression vector for expressing crRNA corresponding to the sequence derived from the human EMX1 gene).

[3] Preparation of Cas3 Expression Vector

A polynucleotide having a modified nucleic acid sequence encoding Cas3 (SEQ ID NO: 1) was obtained from Genscript. Specifically, pUC57 vector incorporating the polynucleotide described above was obtained from Genscript.

The above vector was cleaved with restriction enzyme NotI. Next, 2 U of Klenow Fragment (Takara Bio Inc.) and 1 μL of 2.5 mM dNTP Mixture (Takara Bio Inc.) were used to smooth the edge of the fragment. Thereafter, the above fragment was purified using Gel extraction (Qiagen). The purified fragment was further cleaved with restriction enzyme XhoI and purified using Gel extraction (Qiagen).

The purified fragment was ligated using a substrate plasmid (pTL2-CAG-IRES-NEO vector, prepared by Takeda Laboratory) and a ligation kit (Mighty Mix, Takara Bio Inc.). After that, transformation and purification were carried out by the same operations as in [2]. The recovered plasmid vector was prepared to have a concentration of 1 μg/μL in a TE buffer solution.

[4] Preparation of Plasmid Vector Containing BPNLS

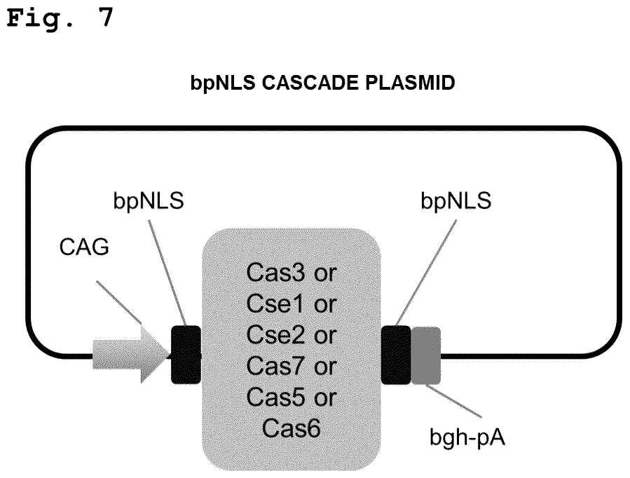

Prepared were Cas3, Cse1 (Cas8), Cse2 (Cas11), Cas5, Cas6, and Cas7 expression vectors in which BPNLSs were connected to the 5′-end and the 3′-end (see FIG. 7).

The production of an insert for each Cas protein group containing BPNLSs at both ends was ordered to Thermo Fisher Scientific. The specific sequence of the above insert is (AGATCTTAATACGACTCACTATAGGGAGAGCCGCCACCATGGCC: SEQ ID NO: 56)-(any one of SEQ ID NOs: 7 to 12)-(TAATATCCTCGAG: SEQ ID NO: 57). SEQ ID NO: 56 is a sequence provided with a cleavage site by BgIII. SEQ ID NO: 57 is a sequence provided with a cleavage site by XhoI.

The pMK vector incorporating the above sequence was cleaved with restriction enzymes BgIII and XhoI and purified using Gel extraction (Qiagen). The purified fragment was ligated using a substrate plasmid (pPB-CAG-EBNXN, supplied from Sanger Center) and a ligation kit (Mighty Mix, Takara Bio Inc.). After that, transformation and purification were carried out by the same operations as in [2]. The recovered plasmid vector was prepared to have a concentration of 1 μg/μL in a TE buffer solution.

[5] Preparation of Plasmid Vector Containing Cascade (2A)

Prepared was an expression vector in which the nucleic acid sequence had Cse1 (Cas8), Cse2 (Cas11), Cas7, Cas5, and Cas6 linked in this order. More specifically, prepared was an expression vector having an arrangement of (NLS-Cse1 (Cas8): SEQ ID NO: 2)-2A-(NLS-Cse2 (Cas11): SEQ ID NO: 3)-2A-(NLS-Cas7: SEQ ID NO: 6)-2A-(NLS-Cas5: SEQ ID NO: 4)-2A-(NLS-Cas6: SEQ ID NO: 5) (see FIG. 8). Note that the amino acid sequence of NLS is PKKKRKV (SEQ ID NO: 52), and the nucleic acid sequence is CCCAAGAAGAAGCGGAAGGTG (SEQ ID NO: 53). In addition, the amino acid sequence of 2A peptide is GSGATNFSLLKQAGDVEENPGP (SEQ ID NO: 58) (the corresponding nucleic acid sequences are SEQ ID NOs: 59 to 62).

A polypeptide having the above nucleic acid sequence was obtained from GenScript. The pUC57 vector incorporating the above sequence was cleaved with restriction enzyme EcoRI-HF and purified using Gel extraction (Qiagen). The purified fragment was ligated using a substrate plasmid (pTL2-CAG-IRES-Puro vector, prepared by Takeda Laboratory) and a ligation kit (Mighty Mix, Takara Bio Inc.). After that, transformation and purification were carried out by the same operations as in [2]. The recovered plasmid vector was prepared to have a concentration of 1 μg/μL in a TE buffer solution.

### Example A-1

The cleavage activity of the target sequence of the exogenous DNA was evaluated as follows. A crRNA and Cas3, Cse1 (Cas8), Cse2 (Cas11), Cas5, Cas6, and Cas7 added with nuclear localization signals to have a modified nucleic acid sequence were expressed in HEK (human embryonic kidney) 293T cells.

Prior to transfection, the HEK 293T cells were cultured in a 10 cm dish. Culture of the HEK 293T cells was carried out in EF medium (GIBCO) at 37° C. in a 5% CO₂ atmosphere. The density of HEK 293T cells in the EF medium was adjusted to 3×10^{4/100} μL.

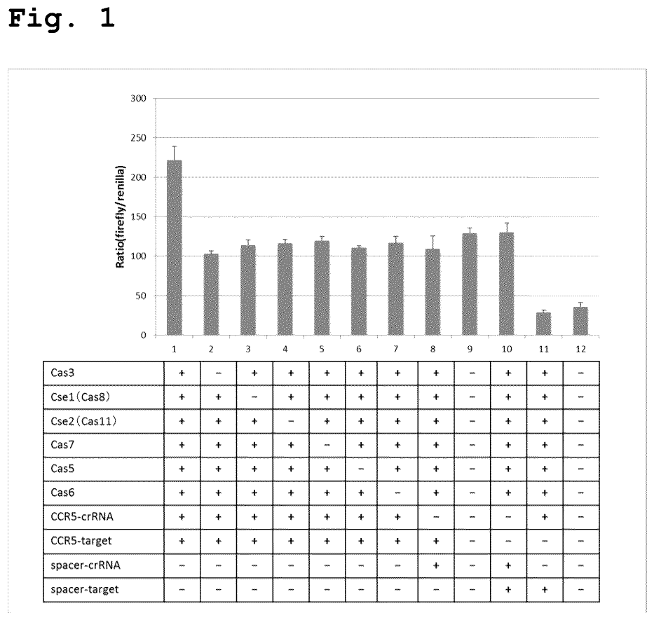

In addition, 100 ng of the above reporter vector; 200 ng of each of the Cas3 plasmid, the Cse1 (Cas8) plasmid, the Cse2 (Cas11) plasmid, the Cas5 plasmid, the Cas6 plasmid, the Cas7 plasmid, and the crRNA plasmid; 60 ng of pRL-TK vector (capable of expressing Renilla luciferase, Promega); and 300 ng of pBluecscript II KS (+) vector (Agilent Technologies) were mixed in 25 μL of Opti-MEM (Thermo Fisher Scientific). The conditions using the reporter vector having the target sequence derived from CCR5 as the reporter vector correspond to 1 in FIG. 1, and the conditions using the reporter vector having the spacer sequence of E. coli CRISPR correspond to 10 in FIG. 1.

Next, 1.5 μL of LIPOFECTAMINE™ 2000 (Thermo Fisher Scientific) and 25 μL of OPTIMEM™ (Thermo Fisher Scientific) were mixed and incubated at room temperature for 5 minutes. Thereafter, the above plasmid+OPTIMEM™ mixture and LIPOFECTAMINE™ 2000+OPTIMEM™ mixture were mixed and incubated at room temperature for 20 minutes. The resulting mixture was mixed with 1 mL of the above EF medium containing HEK 293T cells and seeded in a 96-well plate (seeded in a total of 12 wells, 1 well per combination of vectors).

After culturing at 37° C. in a 5% CO₂ atmosphere for 24 hours, a dual-Luciferase assay was carried out in accordance with the protocol of the DUAL-GLO™ Luciferase assay system (Promega). For measurement of luciferase and Renilla luciferase, CENTRO XS³ LB 960™ (BERTHOLD TECHNOLOGIES) was used.

The same experiment was conducted under the following conditions as a control experiment.
1. Instead of any one of the Cas3 plasmid, the Cse1 (Cas8) plasmid, the Cse2 (Cas11) plasmid, the Cas5 plasmid, the Cas6 plasmid, and the Cas7 plasmid, the same amount of pBluecscript II KS(+) vector (Agilent Technologies) was mixed for expression (2 to 7 in FIG. 1).
2. Instead of the crRNA plasmid used in the above procedure, plasmids for expressing a crRNA not complementary to the target sequence were mixed. Specifically, for the purpose of expression, plasmids for expressing the crRNA corresponding to the spacer sequence of E. coli CRISPR were mixed for the target sequence derived from the CCR5 gene (8 in FIG. 1), and plasmids for expressing the crRNA corresponding to the sequence derived from the CCR5 gene were mixed when targeting the spacer sequence of E. coli CRISPR (11 in FIG. 1).
3. As negative controls, only a reporter vector having the target sequence derived from CCR5 (9 in FIG. 1) and only a reporter vector having the spacer sequence of E. coli CRISPR (12 in FIG. 1) were expressed.

(Results)

The results of the dual-Luciferase assay are shown in the graph of FIG. 1, and the experimental conditions are shown in the lower table of FIG. 1. In FIG. 1, “CCR5-target” and “spacer-target” represent the target sequence derived from CCR5 and the spacer sequence of E. coli CRISPR, respectively. In addition, “CCR5-crRNA” and “spacer-crRNA” represent the sequence complementary to CCR5-target and the sequence complementary to spacer-target, respectively.

In FIG. 1, the system into which the crRNA plasmid complementary to the target sequence and all of the Cas3 plasmid, the Cse1 (Cas8) plasmid, the Cse2 (Cas11) plasmid, the Cas5 plasmid, the Cas6 plasmid, and the Cas7 plasmid were introduced exhibited cleavage activity higher than that of other systems (compare between 1 and 2 to 8, and between 10 and 11). Therefore, it was found that it is possible to express Cas3, Cse1 (Cas8), Cse2 (Cas11), Cas5, Cas6, and Cas7 in human cells by using the expression vectors according to an embodiment of the present invention.

In addition, it was suggested that introducing of the above expression vectors into human cells forms Cas3, Cascade, and crRNA complexes in human cells and cleaves the target sequence.

Furthermore, in FIG. 1, comparison between 8 and 9 and between 11 and 12 reveals that cleavage activity was equivalent to that of the negative controls in a system expressing a crRNA not complementary to the target sequence. In other words, it was suggested that the CRISPR-Cas3 system of the present invention can specifically cleave sequences complementary to crRNA in mammalian cells.

### Example A-2

An experiment was conducted to evaluate whether or not it is possible to cleave endogenous DNA of human cells by type I CRISPR-Cas systems using the same method as in Example A-1.

Specifically, the nucleic acid sequence in human cells was modified to express pre-crRNA and Cas3, Cse1 (Cas8), Cse2 (Cas11), Cas5, Cas6, and Cas7 added with nuclear localization signals, and evaluation was conducted on whether or not the sequence of the endogenous CCR5 gene of the cells would be cleaved.

The same HEK 239T cells as in Example A-1 were seeded in a 24-well plate at a density of 1×10⁵ cells/well and cultured for 24 hours.

Mixed with 50 μL of OPTI-MEM™ (Thermo Fisher Scientific) were 1 μg of the Cas3 plasmid, 1.3 μg of the Cse1 (Cas8) plasmid, 1.3 μg of the Cse2 (Cas11) plasmid, 1.1 μg of the Cas5 plasmid, 0.8 μg of the Cas6 plasmid, 0.3 μg of the Cas7 plasmid, and 1 μg of the crRNA plasmid. Subsequently, a mixture of 5 μL of LIPOFECTAMINE (registered trademark) 2000 (Thermo Fisher Scientific), 50 μL of OPTI-MEM™ Thermo Fisher Scientific), and 1 mL of EF medium was added to the above DNA mixture. Thereafter, 1 mL of the resulting mixture was added to the above 24-well plate.

After culturing at 37° C. in a 5% CO₂ atmosphere for 24 hours, the medium was replaced with 1 mL of EF medium. Past 48 hours following transfection (24 hours after medium replacement), the cells were harvested and adjusted to a concentration of 1×10⁴ cells/5 μL in PBS.

The above cells were heated at 95° C. for 10 minutes. Next, 10 mg of proteinase K was added, followed by incubation at 55° C. for 70 minutes. Furthermore, the product heat-treated at 95° C. for 10 minutes was used as a template for PCR.

By performing 35 cycles of 2-step PCR, 10 μL of the above template was amplified. Here, primers having the sequences of SEQ ID NOs: 47 and 48 were used as primers for PCR. In addition, KOD FX (Toyobo Co., Ltd.) was used as a DNA polymerase, and the 2-step PCR procedure was in accordance with the protocol attached to KOD FX. The product amplified by PCR was purified using QIAQUICK™ PCR Purification Kit (QIAGEN). The specific procedure was in accordance with the protocol attached to the above kit.

The dA was added to the 3′-end of the purified DNA obtained using rTaq DNA polymerase (Toyobo Co., Ltd.). The purified DNA was subjected to electrophoresis in a 2% agarose gel, and a band of about 500 to 700 bp was cut out. Then, DNA was extracted from the cut gel and purified using a Gel extraction kit (QIAGEN). Next, TA cloning was carried out using PGEM-T EASY VECTOR SYSTEMS™ (Promega) and the above DNA was cloned. Finally, DNA cloned by alkaline prep method was extracted and analyzed by Sanger sequence. For the analysis, BIGDYE (registered trademark) Terminator v 3.1 Cycle Sequencing Kit (Thermo Fisher Scientific) and APPLIED BIOSYSTEMS™ 3730 DNA Analyzer (Thermo Fisher Scientific) were used.

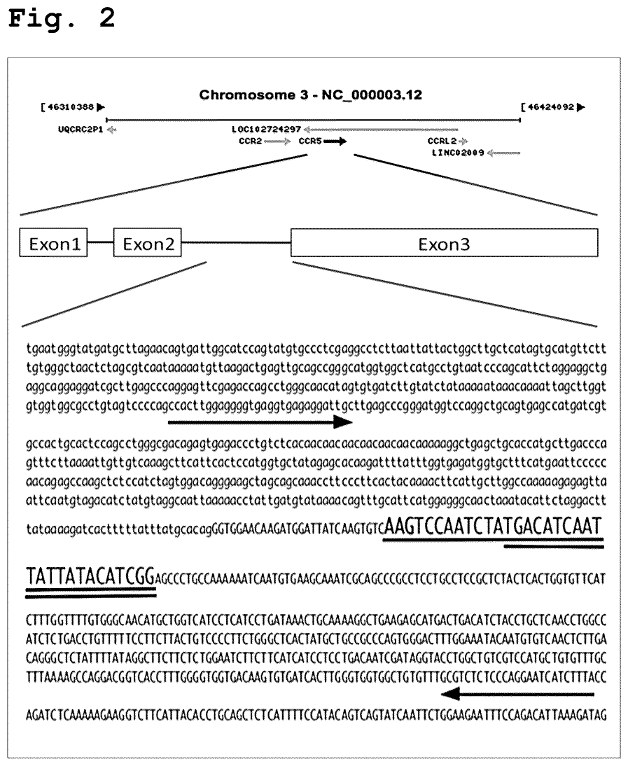

The outline of the endogenous CCR5 gene sequence, the target of the CRISPR-Cas system in this example, is described based on FIG. 2. Note that in FIG. 2, the exons are in capital letters, and the introns are in lowercase letters.

In this example, the target was the sequence within the CCR5 gene located in third chromosome short arm (P) region 21 (FIG. 2; the entire length of the nucleic acid sequence of CCR5 is shown at SEQ ID NO: 46). Specifically, the sequence within Exon 3 of the CCR5 gene was used as the target sequence. As a control, the target sequence of Cas9 was also arranged at approximately the same position. More precisely, the entire underlined sequence is the target sequence of type I CRISPR-Cas system (AAG and the following 32 bases), and the double underlined sequence is the target sequence of Cas9 (CGG and the preceding 20 bases). The sequence of crRNA was designed to allow guidance to the target sequence of the type I CRISPR-Cas system (AAG and the following 32 bases).

(Results)

The results of the above experiment were such that clone 1 having 401 bp deleted, clone 2 having 341 bp deleted, clone 3 having 268 bp deleted, and clone 4 having 344 bp deleted were obtained as compared with the original nucleic acid sequences (FIGS. 3A to 3D). This showed that it is possible to delete the endogenous DNA of human cells by the CRISPR-Cas3 system of the present invention. Specifically, it was suggested that the above CRISPR-Cas system enables editing of DNA of human cells.

This example observed clones having base pairs deleted. This fact supports that DNA cleavage takes place at multiple sites, according to the CRISPR-Cas3 system of the present invention.

DNA of several hundred base pairs (268 to 401 bp) was deleted by the CRISPR-Cas3 system of the present invention. This was more extensive than the deletion obtained by the CRISPR-Cas system using Cas9 (usually cleaved at only one site on the DNA).

### Example A-3

An experiment was conducted to evaluate whether or not it is possible to cleave the endogenous DNA of human cells by the CRISPR-Cas3 system using the same method as in Example A-1.

Specifically, the nucleic acid sequence in human cells was modified to express pre-crRNA and Cas3, Cse1 (Cas8), Cse2 (Cas11), Cas5, Cas6, and Cas7 added with nuclear localization signals, and evaluation was conducted on whether or not the sequence of the endogenous EMX1 gene of the cells would be cleaved.

The same HEK 293T cells as in Example A-1 were seeded in a 24-well plate at a density of 1×10⁵ cells/well and cultured for 24 hours.

Mixed with 50 μL of OPTI-MEM™ (Thermo Fisher Scientific) were 500 ng of the Cas3 plasmid, 500 ng of the Cse1 (Cas8) plasmid, 1 μg of the Cse2 (Cas11) plasmid, 1 μg of the Cas5 plasmid, 1 μg of the Cas6 plasmid, 3 μg of the Cas7 plasmid, and 500 μg of the crRNA plasmid. Further added and mixed in the above mixture were 4 μL of LIPOFECTAMINE (registered trademark) 2000 (Thermo Fisher Scientific) and 50 μL of OPTI-MEM™ (Thermo Fisher Scientific). The resulting mixture was incubated at room temperature for 20 min and then added to the HEK 293T cells.

Here, the structure of the expression vector of the Cas protein group used in Example A-3 is shown in FIG. 7. As shown in FIG. 7, the above expression vector is obtained by sandwiching the sequence encoding the Cas protein group with BPNLSs (bipartite NLSs) (see [Suzuki K et al. (2016) In vivo genome editing via CRISPR/Cas9 mediated homology-independent targeted integration, Nature, Vol. 540 (Issue 7631), pp. 144-149]). The amino acid sequence of BPNLS is KRTADGSEFESPKKKRKVE (SEQ ID NO: 54), and the nucleic acid sequence is

(SEQ ID NO: 55)
AAGCGGACTGCTGATGGCAGTGAATTTGAGTCCCCAAAGAAGAAG
AGAAAGGTGGAA

After the above HEK 293T cells were cultured at 37° C. in a 5% CO₂ atmosphere for 24 hours, the medium was replaced with 1 mL of EF medium (1 mL per 1 well). Past 48 hours following transfection (24 hours after medium replacement), the cells were harvested and adjusted to a concentration of 1×10⁴ cells/5 μL in PBS.

The above cells were heated at 95° C. for 10 minutes. Next, 10 mg of proteinase K was added, followed by incubation at 55° C. for 70 minutes. Furthermore, the product heat-treated at 95° C. for 10 minutes was used as a template for PCR.

By performing 40 cycles of 3-step PCR, 10 μL of the above template was amplified. Here, primers having the sequences of SEQ ID NOs: 50 and 51 were used as primers for PCR. In addition, HOTSTARTAQ™ (QIAGEN) was used as a DNA polymerase, and the 3-step PCR procedure was in accordance with the protocol attached to HOTSTARTAQ™. The product amplified by PCR was subjected to electrophoresis in a 2% agarose gel, and a band of about 900 to 1100 bp was cut out. Then, DNA was extracted from the cut gel and purified using a Gel extraction kit (QIAGEN). The specific procedure was in accordance with the protocol attached to the above kit.

Next, TA cloning was carried out using PGEM-T EASY VECTOR SYSTEMS™ (Promega), and the above DNA was cloned. Finally, DNA cloned by alkaline prep method was extracted and analyzed by Sanger sequence. For the analysis, BIGDYE (registered trademark) Terminator v 3.1 Cycle Sequencing Kit (Thermo Fisher Scientific) and APPLIED BIOSYSTEMS 3730™ DNA Analyzer (Thermo Fisher Scientific) were used.

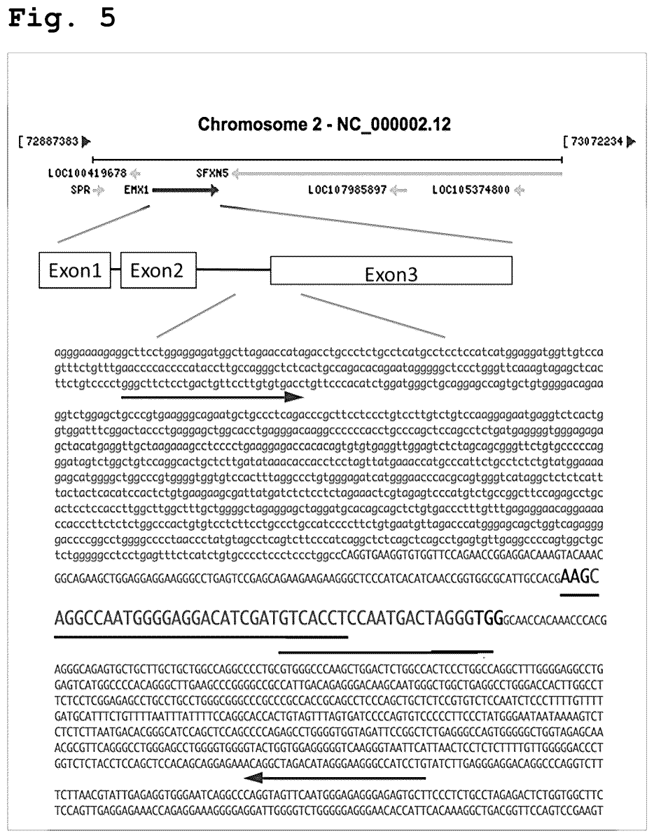

The outline of the endogenous EMX1 gene sequence, the target of the CRISPR-Cas3 system in Example A-3, is described based on FIG. 5. Note that in FIG. 5, the exons are in capital letters, and the introns are in lowercase letters.

In Example A-3, the target was the sequence within the EMX1 gene located in second chromosome short arm (P) region 13 (FIG. 5; the entire length of the nucleic acid sequence of EMX1 is shown at SEQ ID NO: 49). Specifically, the sequence within Exon 3 of the EMX1 gene was used as the target sequence. As a control, the target sequence of Cas9 was also arranged at approximately the same position. More precisely, the underlined sequence located upstream is the target sequence of type I CRISPR-Cas system (AAG and the following 32 bases), and the underlined sequence located downstream is the target sequence of Cas9 (TGG and the preceding 20 bases). The sequence of crRNA used in Example A-3 was designed to allow guidance to the target sequence of the CRISPR-Cas3 system (AAG and the following 32 bases).

(Results)

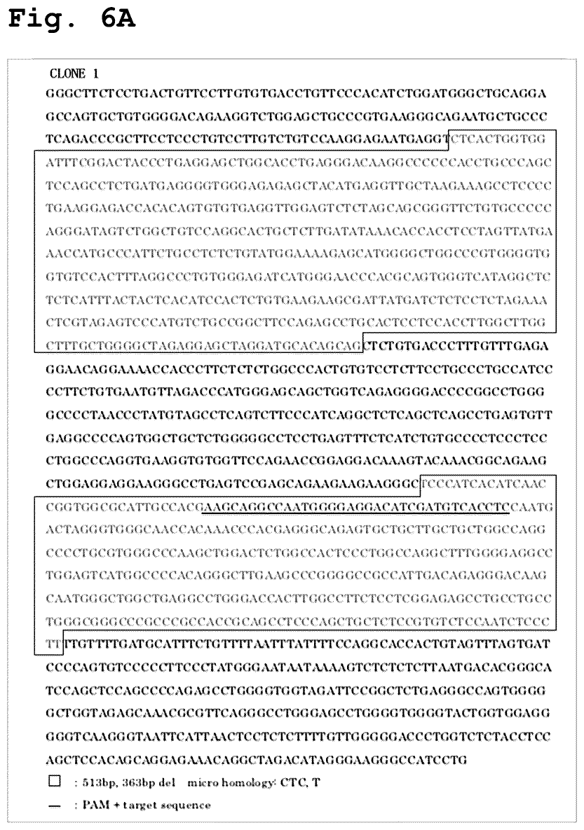

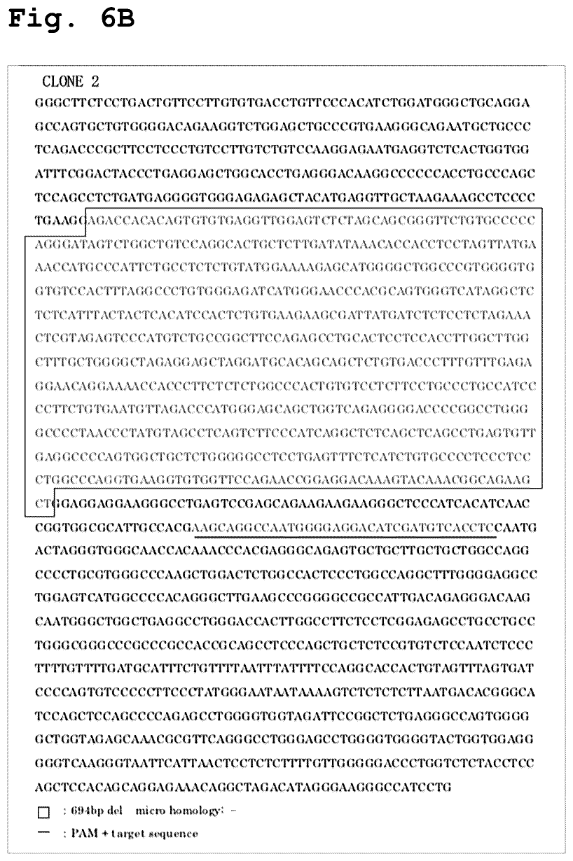

The results of the above experiment were such that clone 1 having two deleted sites of 513 bp and 363 bp and clone 2 having 694 bp deleted were obtained as compared with the original nucleic acid sequences (FIGS. 6A and 6B). These experimental results also showed that it is possible to delete the endogenous DNA of human cells by the CRISPR-Cas3 system of the present invention. Specifically, it was suggested that the above CRISPR-Cas3 system enables editing of DNA of human cells.

In addition, it was similar to Example A-2 that cleavage took place at two or more sites of the double-stranded DNA, and DNA of several hundred base pairs was deleted. Therefore, the results of Example A-3 more strongly support the suggestions obtained from Example A-2.

### Example A-4

The cleavage activity of the target sequence of the exogenous DNA was evaluated as follows. HEK 293T cells were caused to express the CRISPR-Cas3 system in which the nucleic acid sequences were modified and the nucleic acid sequences encoding Cascade proteins were linked.

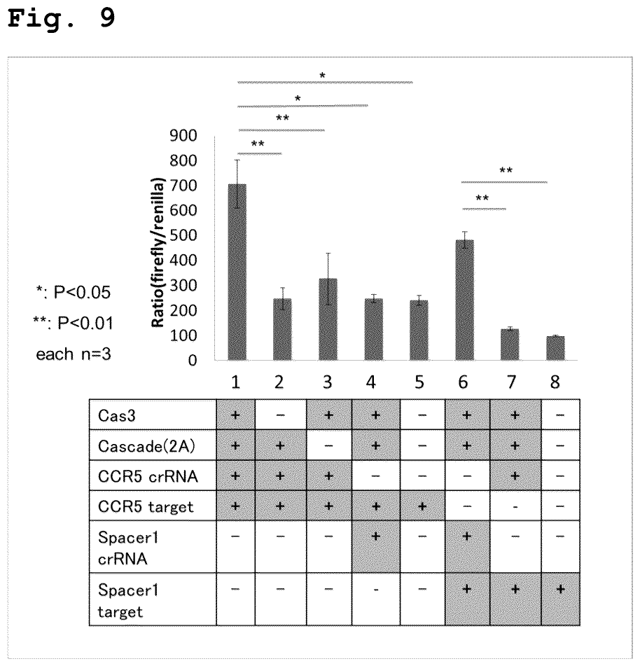

In Example A-4, 100 ng of the reporter vector; 200 ng of each of the Cas3 plasmid, the Cascade (2A) plasmid, and the crRNA plasmid; 60 ng of pRL-TK vector (capable of expressing Renilla luciferase, Promega); and 300 ng of pBluecscript II KS(+) vector (Agilent Technologies) were mixed in 25 μL of Opti-MEM (Thermo Fisher Scientific). The conditions using the reporter vector having the target sequence derived from CCR5 as the reporter vector correspond to 1 in (b) of FIG. 9, and the conditions using the reporter vector having the spacer sequence of E. coli CRISPR correspond to 6 in (b) of FIG. 9.

Here, as the above reporter vectors, the two kinds of reporter vectors prepared in [1] of [Preparation Example] (that is, vectors having the structure shown in FIG. 4D) were used. In addition, as the above Cascade (2A) plasmid, the expression vectors prepared in [4] of [Preparation Example] (that is, the vector having the structure shown in FIG. 8) were used.

A dual-Luciferase assay was carried out in the same method as Example A-1 except that the above expression vectors were used.

Moreover, the same experiment was conducted under the following conditions as a control experiment.
1. Instead of either one of the Cas3 plasmid and the Cascade (2A) plasmid, the same amount of pBluecscript II KS(+) vector (Agilent Technologies) was mixed for expression (2 and 3 in FIG. 9).
2. Instead of the crRNA plasmid used in the above procedure, plasmids for expressing a crRNA not complementary to the target sequence were mixed. Specifically, for the purpose of expression, plasmids for expressing the crRNA corresponding to the spacer sequence of E. coli CRISPR were mixed for the target sequence derived from the CCR5 gene (4 in FIG. 9), and plasmids for expressing the gRNA corresponding to the sequence derived from the CCR5 gene were mixed when targeting the spacer sequence of E. coli CRISPR (7 in FIG. 9)
3. As negative controls, only a reporter vector having the target sequence derived from CCR5 (5 in FIG. 9) and only a reporter vector having the spacer sequence of E. coli CRISPR (8 in FIG. 9) were expressed.

(Results)

The results of the dual-Luciferase assay are shown in the graph of FIG. 9, and the experimental conditions are shown in the lower table of FIG. 9. In FIG. 9, “CCR5-target” and “spacer-target” represent the target sequence derived from CCR5 and the spacer sequence of E. coli CRISPR, respectively. In addition, “CCR5-crRNA” and “spacer-crRNA” represent the sequence complementary to CCR5-target and the sequence complementary to spacer-target, respectively.

As shown in FIG. 9, the system into which the crRNA plasmid complementary to the target sequence and both of the Cas3 plasmid and the Cascade (2A) plasmid were introduced exhibited cleavage activity significantly higher than that of other systems (compare between 1 and 2 to 5, and between 6, 7, and 8). Thus, it was suggested that, even in a system in which nucleic acid sequences encoding Cascade proteins are linked for expression, it is possible to specifically cleave sequences complementary to crRNA in mammalian cells by using the CRISPR-Cas system according to an embodiment of the present invention.

B. Examination of Factors and the Like Affecting Genome Editing by CRISPR-Cas3 System in Eukaryotic Cell

[Material and Method]

[1] Configuration of Cas Gene and crRNA

Constituent genes of Cas3 and Cascade (Cse1, Cse2, Cas5, Cas6, and Cas7) derived from E. coli K-12 strain, to which bpNLSs were added to the 5′ side and the 3′ side, were designed and cloned by codon optimization for mammalian cells followed by gene synthesis. These genes were subcloned downstream of the CAG promoter of the pPB-CAG. EBNXN plasmid donated by Sanger Institute. Mutants of Cas3 such as H74A (dead nickase; dn), K320N (dead helicase; dh), and double mutants of S483A and T485A (dead helicase ver. 2; dh2) were prepared by self-ligation of PCR products of PrimeSTAR MAX. Regarding the crRNA expression plasmid, a sequence of crRNA having two BbsI restriction enzyme sites at the position of the spacer under the U6 promoter was synthesized. All crRNA expression plasmids were prepared by inserting 32-base-pair double-stranded oligos of the target sequence into the BbsI restriction enzyme sites.

The Cas9-sgRNA expression plasmid pX330-U6-Chimeric_BB-CBh-hSpCas9 was obtained from Addgene. Designing of gRNA employed CRISPR web tool, CRISPR design tool, and/or CRISPRdirect to predict unique target sites in the human genome. The target sequence was cloned into the sgRNA scaffold of pX330 in accordance with the protocol of the Feng Zhang laboratory.

The SSA reporter plasmid containing two BsaI restriction enzyme sites was donated by Professor YAMAMOTO Takashi at Hiroshima University. The target sequence of the genomic region was inserted into the BsaI sites. As a Renilla luciferase vector, pRL-TK (Promega) was obtained. All plasmids were prepared by midiprep or maxiprep method using PureLink HiPure Plasmid Purification Kit (Thermo Fisher).

[2] Evaluation of DNA Cleavage Activity with HEK 293T Cells

An SSA assay was carried out as in Example A in order to detect DNA cleavage activity in mammalian cells. HEK 293T cells were cultured at 37° C. in 5% CO₂ with high-Glucose Dulbecco's modified Eagle's medium supplemented with 10% fetal bovine serum (Thermo fisher). In the wells of a 96-well plate, 0.5×10⁴ cells were seeded. After 24 hours, Cas3, Cse1, Cse2, Cas7, Cas5, Cas6, and crRNA expression plasmids (each 100 ng), SSA reporter vectors (100ng), and Renilla luciferase vectors (60ng) were transfected into HEK 293T cells by using LIPOFECTAMINE™ 2000 and OPTIMEM™ (Life Technologies) in accordance with a slightly modified protocol. Twenty four hours after the transfection, a dual luciferase assay was carried out by using the DUAL-GLO™ luciferase assay system (Promega) in accordance with the protocol.

[3] Detection of Indels in HEK 293T Cells

In the wells of a 24-well plate, 2.5×10⁴ cells were seeded. After 24 hours, Cas3, Cse1, Cse2, Cas7, Cas5, Cas6, and crRNA expression plasmids (each 250 ng) were transfected into HEK 293T cells by using LIPOFECTAMINE™ 2000 and OPTIMEM™ (Life Technologies) in accordance with a slightly modified protocol. Two days after the transfection, total DNA was extracted from the harvested cells by using Tissue XS kit (Takara-bio Inc.) in accordance with the protocol. The target locus was amplified by using GFLEX™ (Takara bio Inc.) or QUICK TAQ HS DYEMIX™ (TOYOBO Co., Ltd.), followed by electrophoresis in an agarose gel. For the purpose of detecting small insertion/deletion mutations in PCR products, SURVEYOR™ Mutation Detection Kit (Integrated DNA Technologies) was used in accordance with the protocol. For TA cloning, the pCR4Blunt-TOPO plasmid vector (Life Technologies) was used in accordance with the protocol. For sequence analysis, BIGDYE™ Terminator Cycle Sequencing Kit and ABI PRISM 3130™ Genetic Analyzer (Life Technologies) were used.

For the purpose of detecting various unusual mutations, a DNA library of PCR amplification products was prepared using TRUSEQ™ Nano DNA Library Prep Kit (Illumina), and amplicon sequencing was carried out with MISEQ™ (2×150 bp) in accordance with the standard procedure by Macrogen. The raw reads of the samples were mapped to human genome hg38 by BWA-MEM. The coverage data was visualized with Integrative Genomics Viewer (IGV), and the histogram at the target region was extracted.

Reporter HEK 293T cells having mCherry-P2A-EGFP c321C>G for detecting SNP-KI (snip knock-in) in mammalian cells were donated by Professor NAKADA Shin-ichiro. The reporter cells were cultured with 1 μg/ml of puromycin. Single-stranded DNA or 500 ng of donor plasmid was co-introduced together with CRISPR-Cas3 by the method described above. All cells were harvested 5 days after the transfection, and FACS analysis was carried out using AriaIIIu™ (BD). GFP positive cells were sorted and total DNA was extracted by the method described above. SNP exchange in the genome was detected by PCR amplification using HIDI™ DNA polymerase (myPOLS Biotec).

[4] Detection of Off-Target Site Candidates

Off-target candidates of type I-E CRISPR were detected in human genome hg38 using GGGenome by two different procedures. As PAM candidate sequences, AAG, ATG, AGG, GAG, TAG, and AAC were selected in accordance with existing reports (Leenay, R. T, et al. Mol. Cell 62, 137-147 (2016), Jung, et al. Mol. Cell. 2017 Jung et al., Cell 170, 35-47 (2017)). Positions with fewer mismatches were selected in the first approach for 32 base pairs of the target sequence excluding positions of multiples of 6 because it had been reported that such positions are not recognized as target sites. In the following approach, regions completely matching the 5′-end of the PAM side of the target sequence were detected and listed in descending order.

[5] Deep Sequencing of Off-Target Analysis

In whole genome sequencing, genomic DNA was extracted from the transfected HEK 293T cells and cleaved using the COVARIS™ sonicator. A DNA library was prepared using TRUSEQ™ DNA PCR-Free LT Library Prep Kit (Illumina), and genomic sequencing was carried out using HISEQ™ X (2×150 bp) in accordance with the standard procedure by Takara Bio Inc. The raw reads of the samples were mapped to human genome hg38 by BWA-MEM and cleaned by the Trimmomatic program. Discordant read pairs and split reads were excluded by samtools and Lumpy-sv, respectively. For the purpose of detecting only large deletions in the same chromosome, the read pairs mapped to different chromosomes were removed using BadMateFilter of the Genome Analysis Toolkit program. The total number of the discordant read pairs or split reads in the 100 kb region was counted by Bedtools to calculate the error rate with the negative control. SureSelectXT custom DNA probes were designed with SureDesign under moderately stringent conditions and prepared by Agilent technologies to enrich the off-target candidates before the sequencing. The target regions were selected as follows. The probes near the target regions covered 800 kb upstream and 200 kb downstream of PAM. In the vicinity of off-target regions of CRISPR-Cas3, 9 kb upstream and 1 kb downstream of PAM candidates were covered. In the vicinity of the off-target regions of CRISPR-Cas9, 1 kb of upstream and 1 kb of downstream of PAM were covered. After preparation of the DNA library with SureSelectXT reagent kit and custom probe kit, genome sequencing was carried out with Hiseq 2500 (2×150 bp) in accordance with the standard procedure by Takara Bio Inc. Discordant lead pairs and split leads on the same chromosome were excluded by the method described above. The total number of the discordant read pairs or split reads in the 10 kb region was counted by Bedtools to calculate the error rate with the negative control.

### [Example B-1] Influence of Types of crRNA and Nuclear Localization Signal on DNA Cleavage Activity

In Example A, genomic editing in eukaryotic cells succeeded by chance by using a CRISPR-Cas3 system containing a pre-crRNA (LRSR; leader sequence-repeated sequence-spacer sequence-repeated sequence) as a crRNA. Here, the present inventors assumed that the reason why genome editing in eukaryotic cells using the CRISPR-Cas3 system had not been successful for many years was due to the fact that mature crRNA had been used as crRNA. In light of the above, in addition to the pre-crRNA (LRSR), a pre-crRNA (RSR; repeated sequence-spacer sequence-repeated sequence) and a mature crRNA (5′-handle sequence-spacer sequence-3′-handle sequence) were prepared as crRNAs, and the genome editing efficiency was examined with the reporter system of Example A (FIGS. 10A and 10B). Note that the nucleic acid sequences of the pre-crRNA (LRSR), the pre-crRNA (RSR), and the mature crRNA are shown at SEQ ID NOs: 63, 64, and 65, respectively.

Consequently, no cleavage activity of the target DNA was observed in the CRISPR-Cas3 system using the mature crRNA. On the other hand, it was surprising that, in the case of using the pre-crRNAs (LRSR and RSR), very high cleavage activity of the target DNA was observed. These results in the CRISPR-Cas3 system are in contrast with those of the CRISPR-Cas9 system, which exhibits high DNA cleavage activity by using a mature crRNA. In addition, this fact suggests that use of a mature crRNA is one of the reasons why genomic editing in eukaryotic cells has not succeeded in the CRISPR-Cas3 system.

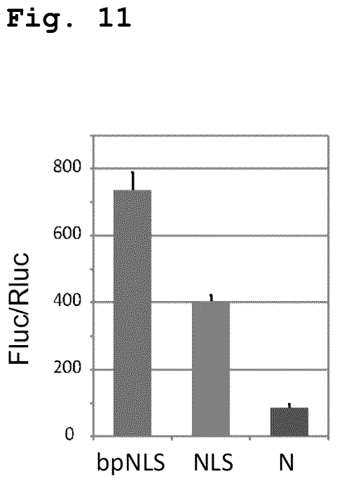

In addition, examination was also carried out using the SV40 nuclear localization signal and bipartite nuclear translocation signal as nuclear localization signals added to Cas3 (FIG. 11). As a result, higher cleavage activity of target DNA was observed when the bipartite nuclear translocation signal was used.

Therefore, in the following experiments, the pre-crRNA (LRSR) was used as a crRNA and the bipartite nuclear translocation signal was used as a nuclear localization signal.

### [Example B-2] Influence of PAM Sequence on DNA Cleavage Activity

For the purpose of confirming the target specificity of the CRISPR-Cas3 system, the effects of various PAM sequences on DNA cleavage activity were examined (FIG. 12). In an SSA assay, the DNA cleavage activity showed various results for different PAM sequences. The highest activity was observed for 5′-AAG PAM, and AGG, GAG, TAC, ATG, and TAG also showed noticeable activity.

### [Example B-3] Influence of Mismatch of crRNA and Spacer Sequence on DNA Cleavage Activity

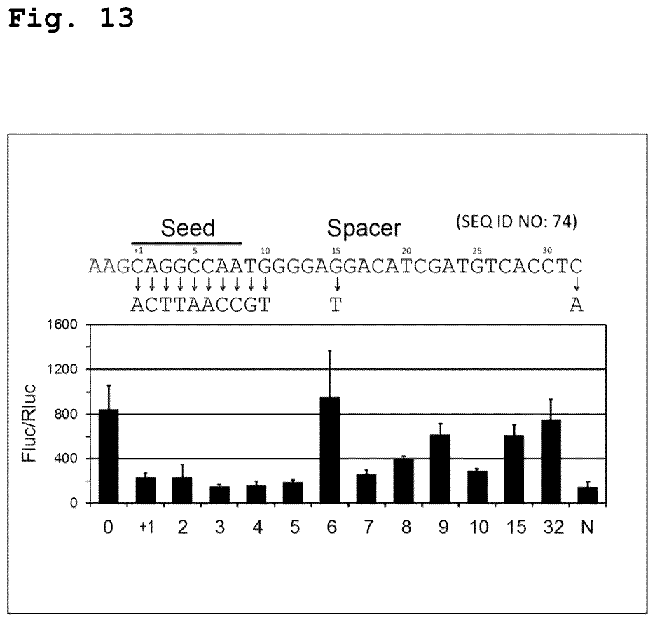

Studies in the past of the crystal structure of E. coli Cascade have shown that a heteroduplex of 5 base partitions is formed between crRNA and spacer DNA. This is due to the failure of base pairing at every sixth position by the SAM element of Cas7 effector (FIG. 13). The influence of mismatch of crRNA and spacer sequence on DNA cleavage activity was evaluated. Cleavage activity dropped dramatically at any single mismatch in the seed region (positions 1-8), except for bases not recognized as a target (position 6).

### [Example B-4] Examination of Necessity of Domains of Cas3 in DNA Cleavage Activity

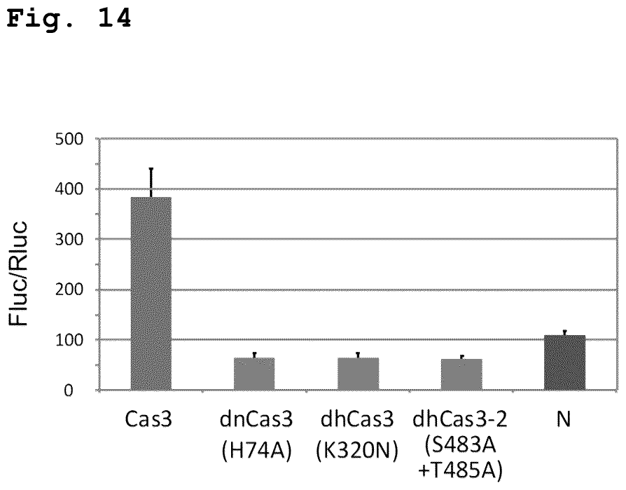

In vitro characterization of the catalytic characteristics of the Cas3 protein revealed that the N-terminus HD nuclease domain cleaves the single-stranded region of the DNA substrate, and subsequently the SF2 helicase domain at the C-terminus unwinds the target DNA in an ATP-dependent manner while proceeding in the 3′- to 5′-direction. Three Cas3 mutants, namely a mutant of HD domain H74A (dnCas3), a mutant of K320N of SF2 domain motif 1 (dhCas3), and a double mutant of S483A/T485A of SF2 domain motif 3 (dh2Cas3) were prepared to examine whether or not the Cas3 domain was necessary for DNA cleavage (FIG. 14). As a result, the DNA cleavage activity completely disappeared in all three mutants of Cas3 protein, revealing that Cas3 can cleave the target DNA through the HD nuclease domain and the SF2 helicase domain.

### [Example B-5] Examination of DNA Cleavage Activity in Various Types of CRISPR-Cas3 Systems

The type 1 CRISPR-Cas3 systems have been highly diversified (A to G of type 1, seven types in total). The above examples examined the DNA cleavage activity in eukaryotic cells in the type I-E CRISPR-Cas3 system. On the other hand, this example examined the DNA cleavage activity in other type 1 CRISPR-Cas3 systems (type I-F and type I-G). Specifically, Cas3 and Cas5-7 of Shewanella putrefaciens of type I-F and Cas5-8 of Pyrococcus furiosus of type I-G were codon optimized and cloned (FIG. 15). As a result, DNA cleavage activity was also found in these type 1 CRISPR-Cas3 systems in the SSA assay using 293T cells although there was a difference in the strength of DNA cleavage activity.

### [Example B-6] Examination of Mutations Introduced into Endogenous Genes by CRISPR-Cas3 System

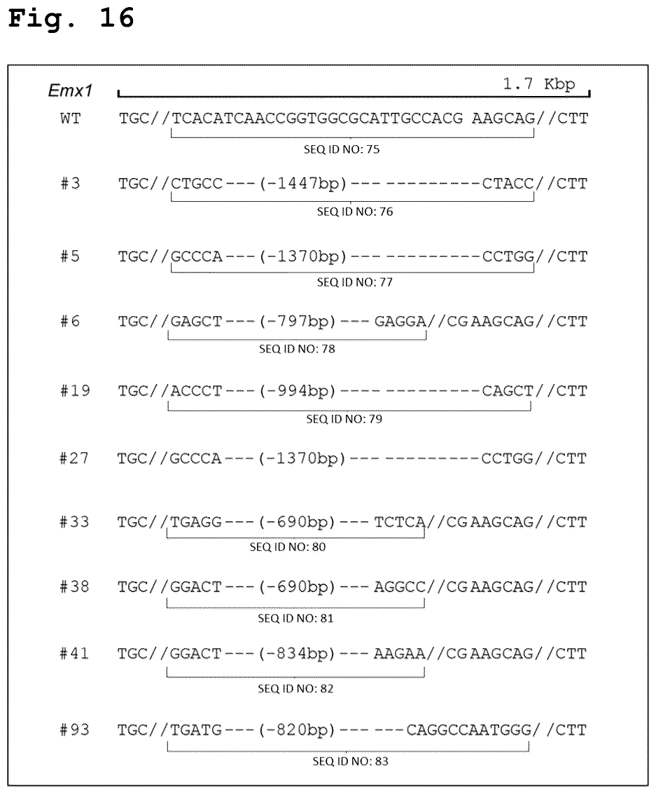

The mutations introduced into endogenous genes by the CRISPR-Cas3 system were examined using the type I-E system. The EMX1 gene and the CCR5 gene were selected as target genes to prepare pre-crRNA (LRSR) plasmids. The 293T cells were lipofected with plasmids encoding pre-crRNA and six Cas (3, 5-8, and 11) effectors. As a result, the CRISPR-Cas3 revealed that deletion of several hundred to several thousand base pairs took place primarily in the upstream direction of the 5′ PAM of the spacer sequence of the target region (FIG. 16). A microhomology of 5 to 10 base pairs at the repaired junction was confirmed, which may have been caused by annealing of the complementary strands by an annealing dependent repair pathway. Note that in the mature crRNA plasmids, no genome editing was found in the EMX1 and CCR5 regions.

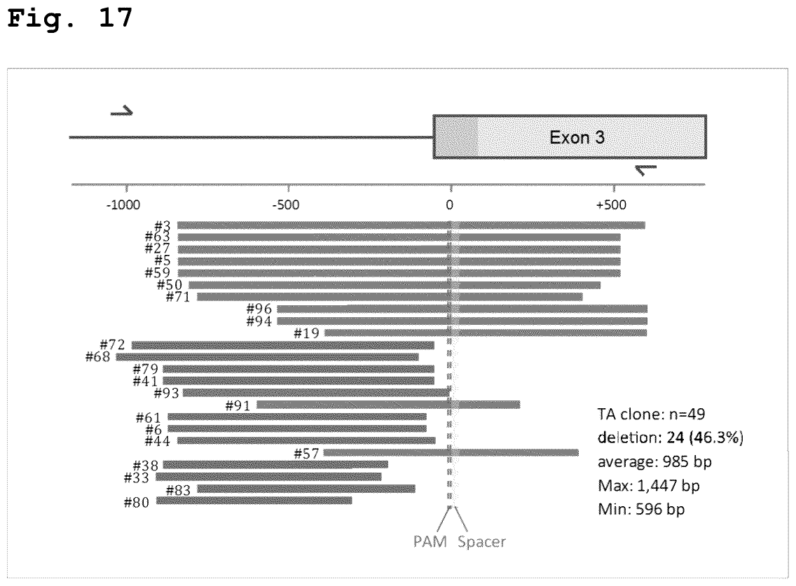

Ninety six TA clones were picked up and compared with sequences of wild type EMX1 by sequencing for the purpose of further characterizing the genome editing by Cas3 by Sanger sequencing and TA cloning of PCR products (FIG. 17). Deletion of a minimum of 596 base pairs, a maximum of 1447 base pairs, and an average of 985 base pairs was observed in 24 clones out of 49 clones which could confirm sequence insertion (efficiency of 46.3%). Half of the clones (n=12) had large deletions including PAM and spacer sequences, and the other half were deleted upstream of PAM.

Further characterization of Cas3 was carried out by next generation sequencing by PCR amplification products with a primer set in broader regions such as 3.8 kb of the EMX1 gene and 9.7 kb of CCR5. Multiple PAM sites (AAG, ATG, and TTT) for targeting with type I-E CRISPR were also examined. In the amplicon sequencing, AAG was 38.2% and ATG was 56.4%. As compared with 86.4% of TTT and 86.4% of Cas9 targeting EMX1, the coverage rate in the broad genomic region upstream of the PAM site was greatly reduced. The decrease in coverage was similar when targeting the CCR 5 region. In contrast, Cas9 induced small insertions and small deletions (indels) at the target sites, while Cas3 had no small indel mutations at PAM or target site. These results suggested that the CRISPR-Cas3 system causes deletions in a wide range upstream of the target site in human cells.

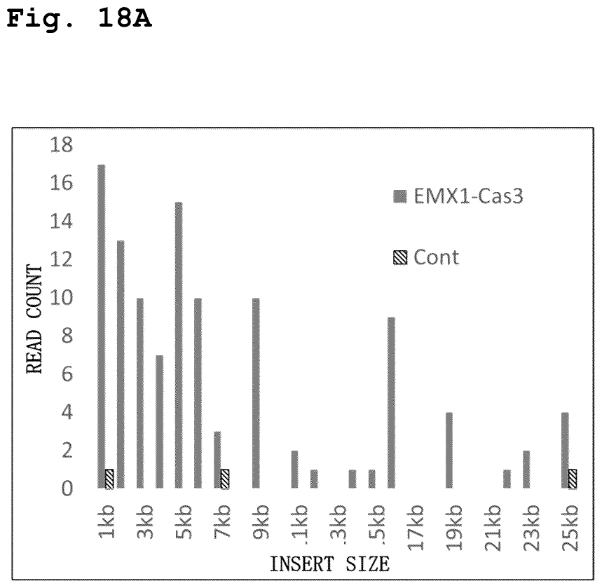

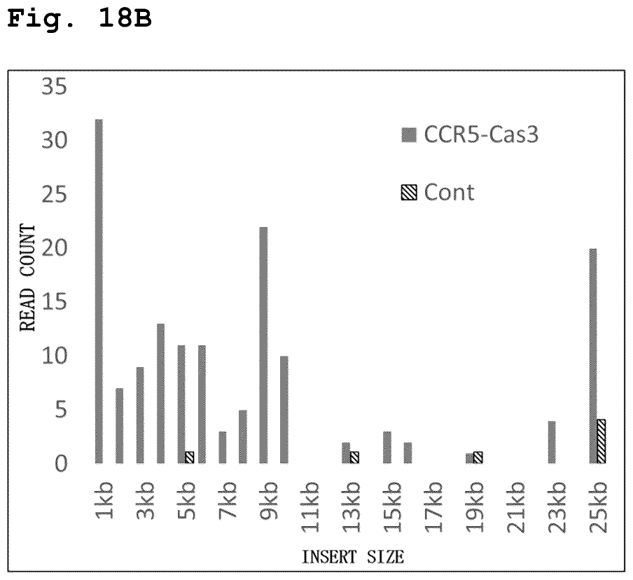

Considering the limitations of PCR analysis such as amplification of less than 10 kb and strong bias favoring shorter PCR fragments, a microarray-based capture sequence of 1000 kb or more around the targeted EMX1 and CCR5 loci was used (FIGS. 18A and 18B). Deletion of up to 24 kb for the EMX1 locus and up to 43 kb for the CCR5 locus was observed. However, 90% of mutations at EMX1 and 95% of mutations at CCR5 were less than 10 kb. These results suggested that the CRISPR-Cas3 system may have potent nuclease and helicase activities in the eukaryotic genome.

It should be noted that whether or not undesirable off-target mutations can be induced in non-target genomic regions is a major concern particularly for clinical applications, as demonstrated in the CRISPR-Cas9 system. However, in the CRISPR-Cas3 system, no significant off-target effects were observed.

### INDUSTRIAL APPLICABILITY

The CRISPR-Cas3 system of the present invention can edit DNA of eukaryotic cells, and therefore can be widely applied to fields requiring genome editing such as medicine, agriculture, forestry, and fisheries, industry, life science, biotechnology, and gene therapy.

1. A method for cleaving endogenous DNA in a eukaryotic cell, comprising:
introducing a CRISPR-Cas3 system that can cleave endogenous DNA in a eukaryotic cell, wherein the CRISPR-Cas3 system is a Type I-D CRISPR-Cas3 system, and wherein the CRISPR-Cas3 system includes the following (A) to (C):
(A) a Cas3 protein, a polynucleotide encoding the protein, or an expression vector containing the polynucleotide,
(B) Cascade proteins comprising Cas10, Cas5, Cas6, and Cas7, a polynucleotide encoding the Cascade proteins, or an expression vector containing the polynucleotide, and
(C) a pre-crRNA which targets the endogenous DNA, a polynucleotide encoding the pre-crRNA, or an expression vector containing the polynucleotide.

2. A method for cleaving endogenous DNA in a nonhuman animal or plant, comprising:
introducing a CRISPR-Cas3 system that can cleave endogenous DNA in a nonhuman animal or plant, wherein the CRISPR-Cas3 system is a Type I-D CRISPR-Cas3 system, and wherein the CRISPR-Cas3 system includes the following (A) to (C):
(A) a Cas3 protein, a polynucleotide encoding the protein, or an expression vector containing the polynucleotide,
(B) Cascade proteins comprising Cas10, Cas5, Cas6, and Cas7, a polynucleotide encoding the Cascade proteins, or an expression vector containing the polynucleotide, and
(C) a pre-crRNA which targets the endogenous DNA, a polynucleotide encoding the pre-crRNA, or an expression vector containing the polynucleotide.

3. The method according to claim 1, further comprising cleaving the pre-crRNA which targets the endogenous DNA with a protein constituting the Cascade proteins after introducing the CRISPR-Cas3 system into the eukaryotic cell.

4. The method according to claim 1, wherein
a nuclear localization signal is added to the Cas3 protein and/or one or more of the Cascade proteins.

5. The method according to claim 4, wherein
the nuclear localization signal is a bipartite nuclear localization signal.

6. A kit for use in the method according to claim 1, the kit comprising the following (A) to (C):
(A) a Cas3 protein, a polynucleotide encoding the protein, or an expression vector containing the polynucleotide,
(B) Cascade proteins comprising Cas10, Cas5, Cas6 and Cas7, a polynucleotide encoding the Cascade proteins, or an expression vector containing the polynucleotide, and
(C) a pre-crRNA which targets the endogenous DNA, a polynucleotide encoding the pre-crRNA, or an expression vector containing the polynucleotide.

7. The kit according to claim 6, wherein
a nuclear localization signal is added to the Cas3 protein and/or one or more of the Cascade proteins.

8. The kit according to claim 7, wherein
the nuclear localization signal is a bipartite nuclear localization signal.
